SEQUENCE LISTING (289 sequences)

SEQ ID NO: 1 - DNA, 21 bp, Bordetella phage BPP-1
tcggggcgcgcggcgtctgtg

SEQ ID NO: 2 - DNA, 11 bp, Bordetella phage BPP-1
ttcttgagtag

SEQ ID NO: 3 - DNA, 11 bp, Bifidobacterium longum
tcgggggccgc

SEQ ID NO: 4 - DNA, 24 bp, Bifidobacterium longum
gcgctcggtcgcacgaaggcgtag

SEQ ID NO: 5 - DNA, 19 bp, Bacteroides thetaiotamicron
tcgggcgtacgggtttggg

SEQ ID NO: 6 - DNA, 18 bp, Bacteroides thetaiotamicron
tgcgttcttcccaagaat

SEQ ID NO: 7 - DNA, 19 bp, Bacteroides thetaiotamicron
tcgggcgtgcgggtttggg

SEQ ID NO: 8 - DNA, 20 bp, Bacteroides thetaiotamicron
tgcgttcttcccaagaatag

SEQ ID NO: 9 - DNA, 17 bp, Vibrio harveyi
tcggttttcgccccgct

SEQ ID NO: 10 - DNA, 21 bp, Treponema denticola
tcttggcttccgcttggcttg

SEQ ID NO: 11 - DNA, 21 bp, Treponema denticola
tcttggcttccgcctggcttg

SEQ ID NO: 12 - DNA, 46 bp, Trichodesmium erythraeum
tctcgtcttccccggtggtttctggctttcattcctagtattcttc

SEQ ID NO: 13 - DNA, 46 bp, Trichodesmium erythraeum
tctcctcttccccggtggtttctggctttcattcctagtattcttc

SEQ ID NO: 14 - DNA, 31 bp, Trichodesmium Erythraeum
ttggttttcgtcttgtgagtttcccccccag

SEQ ID NO: 15 - DNA, 12 bp, Trichodesmium Erythraeum
actcttgaatag

SEQ ID NO: 16 - DNA, 31 bp, Nostoc sp. 7120
ttggttttcgtgttgtctgcgcgttcgggag

SEQ ID NO: 17 - DNA, 30 bp, Nostoc punctiforme
ttggttttcgtgttgtctgcgcgttcggga

SEQ ID NO: 18 - DNA, 10 bp, Nostoc punctiforme
tcttcagtag

SEQ ID NO: 19 - DNA, 28 bp, Nostoc sp. 7120
ggttttcgggttgtggttgtgcggggca

SEQ ID NO: 20 - DNA, 28 bp, Nostoc sp. 7120
ggttgtcgggttgtggttgtgcggggca

SEQ ID NO: 21 - DNA, 24 bp, Chlorobium phaeobacteroides
tcggttttcgtgttgttcgtccca

SEQ ID NO: 22 - DNA, 22 bp, Chlorobium phaeobacteroides
tgcccgttttatggtgcggtaa

SEQ ID NO: 23 - DNA, 19 bp, Chlorobium phaeobacteroides
tcttttgtgattatctgat

SEQ ID NO: 24 - DNA, 21 bp, Chlorobium phaeobacteroides
tcttttgtgattatctgatac

SEQ ID NO: 25 - DNA, 24 bp, Pelodictyon phaeoclathhratiforme
ttggctttcgggttgtccgttcca

SEQ ID NO: 26 - DNA, 23 bp, Pelodictyon phaeoclathhratiforme
gcccctttcgatgcgtgttaaag

SEQ ID NO: 27 - DNA, 23 bp, Pelodictyon phaeoclathhratiforme
tcttcctgatcttctgtctttct

SEQ ID NO: 28 - DNA, 21 bp, Prosthecochloris aestuarii
tttgggcttccgggttgtgag

SEQ ID NO: 29 - DNA, 24 bp, Prosthecochloris aestuarii
tatcgccagatggggattgtttac

SEQ ID NO: 30 - DNA, 21 bp, Prosthecochloris aestuarii
tttgggcttccgccttgtgag

SEQ ID NO: 31 - DNA, 18 bp, Prosthecochloris aestuarii
tagtatcccttggggttt

SEQ ID NO: 32 - DNA, 24 bp, Prosthecochloris aestuarii
tagtatctcttggggtttttacca

SEQ ID NO: 33 - protein, 7 aa, Artificial Sequence
  Synthetic construct
IGXXXSQ

SEQ ID NO: 34 - protein, 7 aa, Artificial Sequence
  Synthetic construct
LGXXXSQ

SEQ ID NO: 35 - DNA, 133 bp, Bordetella phage
cgctgctgcgctattcggcggcaactggaacaacacgtcgaactcgggttctcgcgctgcgaactggaacaacgggccgtcgaactcgaacgcgaacatcggggcgcgcggcgtctgtgcccatcacttcttg

SEQ ID NO: 36 - DNA, 133 bp, Artificial Sequence
  Synthetic construct
cgctgctgcgctattcggcggcaactggaacaacacgtcgaactcgggttctcgcgctaaaaactggaacaacgggccgtcgaactcgaacgcgaacatcggggcgcgcggcgtctgtgcccatcacttcttg

SEQ ID NO: 37 - DNA, 384 bp, Bordetella phage
atggaacccatcgaggaagcgacaaagtgctacgaccaaatgctcattgtggaacggtacgaaagggttatttcgtacctgtatcccattgcgcaaagcatcccgaggaagcacggcgttgcgcgggaatgttcctgaagtgcctgctcgggcaggtcgaattattcatcgtggcgggcaagtccaatcaggtgagcaagctgtacgcagcggacgccgggcttgccatgctgcgattttggttgcgctttctccgggcattcagaaaccgcacgctatgacgccgcatcaggtcgagacagcacaagtgctcatcgccgaagtggggcgcattctcggctcctggattgcccgcgtgaatcgcaaagggcaggctgggaataa

SEQ ID NO: 38 - DNA, 385 bp, Artificial Sequence
  Synthetic construct
atggaacccatcgaggaagcgacatagtgctacgaccaaatgctcattgtggaacggtacgaaagggttatttcgtacctgtatcccattgcgcaaagcatcccgaggaagcacggcgttgcgcgggaatgttcctgaagtgcctgctcgggcaggtcgaattattcatcgtggcgggcaagtccaatcaggtgagcaagctgtacgcagcggacgccgggcttgccatgctgcgattttggttgcgctttctcgcgggcattcagaaaccgcacgctatgacgccgcatcaggtcgagacagcacaagtgctcatcgccgaagtggggcgcattctcggctcctggattgcccgcgtgaatcgcaaagggaggctgggaaataa

SEQ ID NO: 39 - protein, 264 aa, Aminoglycoside-3′-phosphotransferase-II (APH(3′)-IIa) [sequence omitted]

SEQ ID NO: 40 - DNA, 133 bp, APH(3′)-IIa
aacctcgtgaattacggtaacgccgctcccgataagcagcgcatcgccaactatcgccttcttgacaagaacttctgatcgaactcgaacgcgaacatcggggcgcgcggcgtctgtgcccatcaccttcttg

SEQ ID NO: 41 - DNA, 133 bp, Artificial Sequence
  Synthetic construct
ttcctcgtgctttaaggtatcgccgctcccgattcgcagcgcatcgccttctatcgccttcttgacgagttcttctgatcgttctcgttcgcgttcttcggggcgcgcggcgtctgtgaccacctgattcttg

SEQ ID NO: 42 - DNA, 133 bp, Artificial Sequence
  Synthetic construct
ttcctcgtgctttacggtatcgccgctcccgattcgcagcgcatcgccttctatcgccttcttgacgagttcttctgatcgttctcgttcgcgttcttcggggcgcgcggcgtctgtgaccacctgattcttg

SEQ ID NO: 43 - DNA, 133 bp, Artificial Sequence
  Synthetic construct
ttcctcgtgcttttcggtatcgccgctcccgattcgcagcgcatcgccttctatcgccttcttgacgagttcttctgatcgttctcgttcgcgttcttcggggcgcgcggcgtctgtgaccacctgattcttg

SEQ ID NO: 44 - protein, 329 aa, Treponema denticola
MKNTNSKLKTKVLNRAISITALLLAAGVLLTGCPTGQGKSGGGESSEVTPNTPVDKTYTVGSVEFTMKGIAAVNAQLGHNDYSINQPHTVSLSAYLIGETEVTQELWQAVMGNNPSHFNGSPAVGETQGKRPVENVNWYQAIAFCNKLSIKLNLEPCYTVNVGGNPVDFAALSFDQIPDSNNADWDKAELDINKKGFRLPTEAEWEWAAKGGTDDKWSGTNTEAELKNYAWYGSNSGSKTHEVKKKKPNWYGLYDIAGNVAEWCWDWRADIHTGDSFPQDYPGPASGSGRVLRGGSWAGSADYCAVGERVNISPGVRCSDLGFRLACRP

SEQ ID NO: 45 - DNA, 136 bp, Treponema denticola
ccgcgtcaggctctaaccgtgttaaacgcggcggcagctggaacaacaacgcgaacaactgcactgtaggcaaacggaataacaacagtcctgacaacaggaacaacaatcttggcttccgcttggcttgtcggcc

SEQ ID NO: 46 - DNA, 136 bp, Artificial Sequence
  Synthetic construct
ccgcgtcaggctctggccgtgttttacgcggcggcagctgggccggcagcgcggactactgcgctgtaggcgaacgggtcaacatcagtcctggcgtcaggtgcagcgatcttggcttccgcctggcttgccggcc

SEQ ID NO: 47 - DNA, 136 bp, Artificial Sequence
  Synthetic construct
ccgcgtcaggctctggccgtgttttacgcggcggcagctgggccaactacgcggagggctgcactgtgggcacccggggtaacggcaaccctggcaacaggggcaaccttcttggcttccgcctggcttgccggcc

SEQ ID NO: 48 - DNA, 29 bp, Artificial Sequence
  Synthetic construct
ccctctagagctccggttgcttgtggacg

SEQ ID NO: 49 - DNA, 25 bp, Artificial Sequence
  Synthetic construct
agcaagcttcctcgatgggttccat

SEQ ID NO: 50 - DNA, 38 bp, Artificial Sequence
  Synthetic construct
atatctagacgttttcttgggtctaccgtttaatgtcg

SEQ ID NO: 51 - DNA, 36 bp, Artificial Sequence
  Synthetic construct
ataaagcttcgacattaaacggtagacccaagaaaa

SEQ ID NO: 52 - DNA, 27 bp, Artificial Sequence
  Synthetic construct
aaatctagatctgtctgcgtttgtgtt

SEQ ID NO: 53 - DNA, 27 bp, Artificial Sequence
  Synthetic construct
agcaagcttagcacaggaacacaaacg

SEQ ID NO: 54 - DNA, 28 bp, Artificial Sequence
  Synthetic construct
ccctctagaattccaggcgctggctttc

SEQ ID NO: 55 - DNA, 26 bp, Artificial Sequence
  Synthetic construct
agcggatccgaagcaggacagaaccg

SEQ ID NO: 56 - DNA, 26 bp, Artificial Sequence
  Synthetic construct
agcggatccacctattgaggaaaggc

SEQ ID NO: 57 - DNA, 31 bp, Artificial Sequence
  Synthetic construct
aaatctagacgctgctgcgctattcggcggc

SEQ ID NO: 58 - DNA, 36 bp, Artificial Sequence
  Synthetic construct
atatctagacgggccgtcgaagtcgacgttttcttg

SEQ ID NO: 59 - DNA, 34 bp, Artificial Sequence
  Synthetic construct
ataaagcttcgcgttcgaggtcgacattaaacgg

SEQ ID NO: 60 - DNA, 18 bp, Artificial Sequence
  Synthetic construct
aggtcgacattaaacggt

SEQ ID NO: 61 - DNA, 36 bp, Artificial Sequence
  Synthetic construct
gtcgacgttttcttgggtctaccgtttaatgtcgac

SEQ ID NO: 62 - DNA, 16 bp, Artificial Sequence
  Synthetic construct
cctaggccgcgggccc

SEQ ID NO: 63 - DNA, 16 bp, Artificial Sequence
  Synthetic construct
cctaggtaccgggccc

SEQ ID NO: 64 - DNA, 16 bp, Artificial Sequence
  Synthetic construct
cctagatatcggtctc

SEQ ID NO: 65 - DNA, 32 bp, Artificial Sequence
  Synthetic construct
agatctgtctgcgtttgtgttcctgtgctagc

SEQ ID NO: 66 - DNA, 180 bp, Artificial Sequence
  Synthetic construct
acggccaacactggcggccgcggatcggtgtacgcccagcccgctgctgcgctattcggcggcgcctggaacggcacgtcgctctcgggttctcgcgctgcgctctggtacagcgggccgtcgttctcgttcgcgttcttcggggcgcgcggcgtctgtgaccacctgattcttgagtag

SEQ ID NO: 67 - DNA, 35 bp, Artificial Sequence
  Synthetic construct
sssssssssssssstctgtgaccacctgattcttg

SEQ ID NO: 68 - DNA, 35 bp, Artificial Sequence
  Synthetic construct
sssssssssssssstctgtgcccatcaccttcttg

SEQ ID NO: 69 - DNA, 23 bp, Bordetella phage
aaaaaaaaaaaaaaaaaaaaaaa

SEQ ID NO: 70 - DNA, 23 bp, Artificial Sequence
  Synthetic construct
tgcaagggctcttaagttttttt

SEQ ID NO: 71 - DNA, 23 bp, Artificial Sequence
  Synthetic construct
actaaatactttaatctataaga

SEQ ID NO: 72 - DNA, 23 bp, Artificial Sequence
  Synthetic construct
acctaagaccgcagaaaaaaaaa

SEQ ID NO: 73 - DNA, 23 bp, Artificial Sequence
  Synthetic construct
acgcgaaatatctctaaaaaaaa

SEQ ID NO: 74 - DNA, 23 bp, Artificial Sequence
  Synthetic construct
actctcaacatcaaaaaaaaaaa

SEQ ID NO: 75 - DNA, 23 bp, Artificial Sequence
  Synthetic construct
agcgcaaaaagcaaaaaaaaaaa

SEQ ID NO: 76 - DNA, 23 bp, Artificial Sequence
  Synthetic construct
agccgaaatatctctaacaaaaa

SEQ ID NO: 77 - DNA, 23 bp, Artificial Sequence
  Synthetic construct
agcgcagaaagcaaaaaaaaaaa

SEQ ID NO: 78 - DNA, 23 bp, Artificial Sequence
  Synthetic construct
acgagaaaaaagagcaaaaaaaa

SEQ ID NO: 79 - DNA, 23 bp, Artificial Sequence
  Synthetic construct
accgaaaatcacaaaataaaaaa

SEQ ID NO: 80 - DNA, 100 bp, Bordetella phage
cgctgctgcgctattcggcggcaactggaacaacacgtcgaactcgggttctcgagctgcgaactggaacaacgggccgtcgaactcgaacgcgaacatc

SEQ ID NO: 81 - DNA, 100 bp, Artificial Sequence
  Synthetic construct
cgctgctgcgcttttcggcggcgcctgggccaacacgtcgagctcgggttctcgggctgcggtctggtcctacgggccgtcgctctcgtacgcgtacatc

SEQ ID NO: 82 - DNA, 100 bp, Artificial Sequence
  Synthetic construct
cgctgctgcgctattcggcggcgcctggtacgccccgtcgttctcgggttctcgggctgcgtactggtacgccgggccgtcgtactcggtcgcgagcatc

SEQ ID NO: 83 - DNA, 100 bp, Artificial Sequence
  Synthetic construct
cgctgctgcgctgttcggcggcagctggcactacacgtcgaactcgggttctcgggctgcgatctggtactacgggccgtcgttctcgggcgcgagcgtc

SEQ ID NO: 84 - DNA, 100 bp, Artificial Sequence
  Synthetic construct
cgctgctgcgctattcggcggctcctggtacaacacgtcgaactcgggttctcgtgctgcgtactggtacaacgggccgtcgttctcgttcgcgttcttc

SEQ ID NO: 85 - DNA, 100 bp, Artificial Sequence
  Synthetic construct
cgctgctgcgctattcggcggcagctggagcaacacgtcgaactcgggttctcgggctgcgtactggaacagcgggccgtcgttctcgttcgcgttcttc

SEQ ID NO: 86 - DNA, 100 bp, Bordetella phage
cgctgctgcgctattcggcggcgcctggaccaacacgtcgaactcgggttctcgcgctgcgaactggaacaacgggccgtcgaactcgaacgcgaacatc

SEQ ID NO: 87 - DNA, 100 bp, Artificial Sequence
  Synthetic construct
cgctgctgcgctattcggcggcgcctggtccaacacgtcgaactcgggttctcgcgctgcgtactggccctacgggccgtcgaactcgcacgcgtacgtc

SEQ ID NO: 88 - DNA, 100 bp, Artificial Sequence
  Synthetic construct
cgctgctgcgctattcggcggcgcctggtacaacacgtcgaactcgggttctcgcgctgcgttctggtccagcgggccgtcgtactcgtacgcgagcatc

SEQ ID NO: 89 - DNA, 100 bp, Artificial Sequence
  Synthetic construct
cgctgctgcgctattcggcggcgcctggatcaacacgtcgaactcgggttctcgcgctgcgctctggtactacgggccgtcggtctcgatcgcgaacatc

SEQ ID NO: 90 - DNA, 100 bp, Artificial Sequence
  Synthetic construct
cgctgctgcgctattcggcggcgcctggaactacacgtcgaactcgggttctcgcgctgcgatctggtactacgggccgtcgaactcgaacgcgagcatc

SEQ ID NO: 91 - DNA, 100 bp, Artificial Sequence
  Synthetic construct
cgctgctgcgctattcggcggcgcctgggcctacacgtcgatctcgggttctcgcgctgcgtactggagccacgggccgtcgaactcggccgcgagcatc

SEQ ID NO: 92 - DNA, 52 bp, Bordetella phage
aactggaacaacgggccgtcgaactcgaacgcgaacatssssssssssssss

SEQ ID NO: 93 - DNA, 52 bp, Artificial Sequence
  Synthetic construct
ctctggtacagcgggccgtcgttctccttcgcgttcttssssssssssssss

SEQ ID NO: 94 - DNA, 52 bp, Artificial Sequence
  Synthetic construct
ctctggtacagcgggccgtcgttctcgggcgcgagcctssssssssssssss

SEQ ID NO: 95 - DNA, 52 bp, Artificial Sequence
  Synthetic construct
ctctggtacagcgggccgtcgttctcgtacgcgggcatssssssssssssss

SEQ ID NO: 96 - DNA, 52 bp, Artificial Sequence
  Synthetic construct
ctctggtacagcgggccgtcgtactcgtacgcgagcgtssssssssssssss

SEQ ID NO: 97 - DNA, 52 bp, Artificial Sequence
  Synthetic construct
ctctggtacagcgggccgtcgaactcgtacgcgtacatssssssssssssss

SEQ ID NO: 98 - DNA, 134 bp, Bordetella phage
cgctgctgcgctattcggcggcaactggaacaacacggcgaactcgggttctcgcgctgcgaactggaacaacgggccgtcgaactcgaacgcgaacatcggggcgcgcggcgtctgtgcccatcaccttcttg

SEQ ID NO: 99 - DNA, 139 bp, Artificial Sequence
  Synthetic construct
cgctgctgcgctattcggcggcgcctggaacggcgcggcgctctcgggttctcgcgctgcgctctggtacagcgggccgtcgttctcgttcgcgttcttcggggcgcgcggcgtctgtgaccacctgattcttgagtag

SEQ ID NO: 100 - DNA, 124 bp, Vibrio harveyi
accgattcccgcttcgcgggggcaactggaacaatggctcgaacgccgggctgggcgcgctcaatctgaacaatgcgcggtcgaactcgaacaatagcatcggttttcgccccgctcttgatgt

SEQ ID NO: 101 - DNA, 128 bp, Artificial Sequence
  Synthetic construct
accggttcccgcttcgcgggggctactggaacaatggctcgagcgccgggctgggcgcgctctatctgagctatgcgcggtcgaactcgaacagtagcatcggttttcgccccgctttctttgtgtaa

SEQ ID NO: 102 - DNA, 110 bp, Bifidobacterium longum
ggtgcagcgcttcggcaacctcaggaacggggctgcctgcggcgccttcgccgtgaacctcacgaacgacctcgcgaatcgcaggtggaacatcgggggccgcatatccg

SEQ ID NO: 103 - DNA, 133 bp, Artificial Sequence
  Synthetic construct
ggtgcggcgcttcggcctcctctgggacggggctgcctgcggcgccttcgccgtgtacctcgcgaacgccctcgcgaatcgctggtggcacctcgggggccgcctttctgcgctcggtcgcacgaaggcgtag

SEQ ID NO: 104 - DNA, 190 bp, Bacteroides thetaiotamicron
ttccctgcgtcggggtatcgcaactattccaatggcggggcgaacaacgttggcagctacggctactgttggtcggcggttccgaacaaccagaacaacggtcgcaacctgaacttcaactcgtcgaacgtgaacccgttgaacaacaacaatcgggcgtacgggtttggggtgcgttcttcccaagaat

SEQ ID NO: 105 - DNA, 192 bp, Artificial Sequence
  Synthetic construct
ttccctgcgtcggggtctcgcgactgttccggtggcggggcgaacagcgttggcttctacggcgtctgttggtcggcggttccgtacagccagtaccacggttgcaccctggacttctcctcgtcgtccgtgtacccgttgctctactactctcgggcgtgcgggtttgggttgcgttcttcccaagaatag

SEQ ID NO: 106 - DNA, 137 bp, Treponema denticola
ccgcgtcaggctctaaccgagttaaacgcggcggcagctggaacaacaacgcgaacaactgcactgtaggcaaacggaataacaacagtcctgacaacaggaacaacaatcttggcttccgcttggcttgtcggccc

SEQ ID NO: 107 - DNA, 140 bp, Artificial Sequence
  Synthetic construct
ccgcgtcaggctctggccgtgttttacgcggcggcagctgggccggcagcgcggactactgcgctgtaggcgaacgggtcaacatcagtcctggcgtcaggtgcagcgatcttggcttccgcctggcttgccggccttaa

SEQ ID NO: 108 - DNA, 121 bp, Trichodesmium erythraeum
gcggctcctggaacaactatcctaggaggtgtcgctctgcgaaccgcaacaactataactcggacgaggcggacaacaacaatattggttttcgtcttgtgagtttccccccagcactctt

SEQ ID NO: 109 - DNA, 127 bp, Artificial Sequence
  Synthetic construct
gcggctcctggctcaactatccttggtggtgtcgctctgcgtaccgctacgactttagctcggacggggcggtcatcatcaattttggttttcgtcttgtgagtttccccccaggactcttgaatag

SEQ ID NO: 110 - DNA, 123 bp, Artificial Sequence
  Synthetic construct
gcggctcctggtacgactttccttggtggtgtcgctctgcgttccgcggctactatttctcggtcgaggcggtcaacgactttgttggttttcgtcttgtgagtttccccccaggactcctga

SEQ ID NO: 111 - DNA, 150 bp, Trichodesmium erythraeum
gctccgtggcggtagctggaaccacaattctagacattgccggagtgccaggggcaactataaaaatgccgacaacctcaacaacaataggggttttcgagtcatctcgtcttccccggtggtttctggctttcattcctagtattcttc

SEQ ID NO: 112 - DNA, 150 bp, Artificial Sequence
  Synthetic construct
gctccgtggcggttgctggatccacaattctagattttgccggagtgcctggcgcaactatctctatgccgactacctctccaacgataggggttttcgagtcatctcctcttccccggtggtttctggctttcattcctagtattcttc

SEQ ID NO: 113 - DNA, 138 bp, Nostoc spp. 7120
ctgcggggcggctcctggaacaacaatcctgaaaactgccgttccgcgtcccgcaacaacaacaatagggcggagcgcgacaacatcaacaacaatattggttttcgtgttgtctgcgcgttcgggagtactcttcac

SEQ ID NO: 114 - DNA, 141 bp, Artificial Sequence
  Synthetic construct
ctgcggggcggctcctgggacgaccttcctgaaggctgccgttccgcgtcccgcctcagcctcaatagggcggtgcgcgacctcatcctctacagttttggttttcgtgttgtctgcgcgttcgggaggattcttcagtag

SEQ ID NO: 115 - DNA, 141 bp, Artificial Sequence
  Synthetic construct
ctgcggggcggctcctggagctcctctcctgtagtctgccgttccgcgtcccgcggcaacaacgatagggcggggcgcgtctaccgctactacgctgttggttttcgtgttgtctgcgcgttcgggaggacttttcagtag

SEQ ID NO: 116 - DNA, 122 bp, Nostoc spp. 7120
ctgctgcgcggtggttcgtggaacaacaatcccaggaattgccgttcggcgaatcgcaacaggaacgcgcgtgacaacaggaacaacaacgtaggttttcgggttgtggttgtgcggggcag

SEQ ID NO: 117 - DNA, 132 bp, Artificial Sequence
  Synthetic construct
ctgctgcgtggtggttcgtggaactactatcccaggggttgccgttcgttgagtcgcctcagtaacacgcgcgacgacaggaacgagcgcgtgggttgtcgggttgtggttgtgcggggcaggctttcttag

SEQ ID NO: 118 - DNA, 137 bp, Nostoc Punctiforme
ctgcggggcggttcctggatcaacaatcctaaaaactgccgttccgcgtcccgcaacaacaacaatagggcggagcgcgacaacatcaacaacaatattggttttcgtgttgtctgcgcgttcgggatgtctcttca

SEQ ID NO: 119 - DNA, 141 bp, Artificial Sequence
  Synthetic construct
ctgcggggcggttcctggttcaacaatcctgatttctgccgttccgcgtcccgcgtcatcaacagttgggcggagcgcgacaacgtcgtcagcaatgttggttttcgtgttgtctgcgcgttcgggaggattcttcagtag

SEQ ID NO: 120 - DNA, 141 bp, Artificial Sequence
  Synthetic construct
ctgcggggcggttcctggatcttcgatcctgattactgccgttccgcgtcccgcaacctcagctatagggcggagcgcgacggcatcctcagcactcttggttttcgtgttgtctgcgcgttcgggaggattcttcagtag

SEQ ID NO: 121 - DNA, 133 bp, Artificial Sequence; identical to SEQ ID NO: 42
  Synthetic construct

SEQ ID NO: 122 - protein, 25 aa, Artificial Sequence
  Synthetic construct
FLVLYGIAAPDSQRIAFYRLLDEFF

SEQ ID NO: 123 - protein, 18 aa, Artificial Sequence
  Synthetic construct Hypothetical encoded peptide sequence
SFSFAFFGARGVCDHLIL

SEQ ID NO: 124 - DNA, 137 bp, Nostoc punctiforme; identical to SEQ ID NO: 118

SEQ ID NO: 125 - DNA, 138 bp, Nostoc spp. 7120; identical to SEQ ID NO: 113

SEQ ID NO: 126 - DNA, 122 bp, Nostoc spp. 7120; identical to SEQ ID NO: 116

SEQ ID NO: 127 - DNA, 122 bp, Trichodesmium Erythraeum
gcggctcctggaacaactatcctaggaggtgtcgctctgcgaaccgcaacaactataactcggacgaggcggacaacaacaatattggttttcgtcttgtgagtttcccccccagcactctt

SEQ ID NO: 128 - DNA, 67 bp, Trichodesmium Erythraeum
acaataggggttttcgagtcatctcgtcttccccggtggtttctggctttcattcctagtattcttc

SEQ ID NO: 129 - DNA, 134 bp, Chlorobium phaeobacteroides
ccttttcgttgcgcgggggttcgtggaacaacaaatctgccaatctgtcttgctctgcccggaacaacaacaatccggacaacaggaacaacaatatcggttttcgtgttgttcgtcccaatcatgcccgtttt

SEQ ID NO: 130 - DNA, 140 bp, Pelodictyon phaeoclathratiforme
gatctgcccgtgcgttgcgcggcggttcctggaacaataatcccgacaatctgcgttgctctgcccggaacaacaatcatccggcgaacaggaacaacaatattggctttcgggttgtccgttccaatcatgcccctttc

SEQ ID NO: 131 - DNA, 141 bp, Nostoc punctiforme; identical to SEQ ID NO: 119

SEQ ID NO: 132 - DNA, 141 bp, Nostoc spp. 7120; identical to SEQ ID NO: 114

SEQ ID NO: 133 - DNA, 141 bp, Nostoc punctiforme; identical to SEQ ID NO: 120

SEQ ID NO: 134 - DNA, 141 bp, Nostoc spp. 7120; identical to SEQ ID NO: 115

SEQ ID NO: 135 - DNA, 132 bp, Nostoc spp. 7120; identical to SEQ ID NO: 117

SEQ ID NO: 136 - DNA, 128 bp, Trichodesmium Erythraeum
gcggctcctggctcaactatccttggtggtgtcgctctgcgtaccgctacgactttagctcggacggggcggtcatcatcaattttggttttcgtcttgtgagtttcccccccaggactcttgaatag

SEQ ID NO: 137 - DNA, 124 bp, Trichodesmium Erythraeum
gcggctcctggtacgactttccttggtggtgtcgctctgcgttccgcggctactatttctcggtcgaggcggtcaacgactttgttggttttcgtcttgtgagtttcccccccaggactcctga

SEQ ID NO: 138 - DNA, 67 bp, Trichodesmium Erythraeum
acgataggggttttcgagtcatctcctcttccccggtggtttctggctttcattcctagtattcttc

SEQ ID NO: 139 - DNA, 134 bp, Chlorobium phaeobacteroides
ccttttcgttgcgcgggggttcgtggggcaacaaatctgccaatctgtcttgctctgcccgggtcgtcgtcaatccggacctcaggaacggcgttatcggttttcgtgttgttcgtcccagtcatcttttgtga

SEQ ID NO: 140 - DNA, 134 bp, Chlorobium phaeobacteroides
ccttttcgttgcgcgggggttcgtggctcaacttatctgccaatctgtcttgctctgcccggatcttcgtcaatccggtcagcaggaactacggtttcggttttcgtgttgttcgtcccagtcatcttttgtga

SEQ ID NO: 141 - DNA, 140 bp, Pelodictyon phaeoclathratiforme
aatctgcccgtgcgttgcgcggcggttcctggagcaagtatcccgacggtctgcgttgctctgcccggtacgaccttcatccggcgcacaggagcggcaatgttggctttcgggttgtccgttccagtccctcttcctga

SEQ ID NO: 142 - DNA, 42 bp, Bordetella phage
cgaacatcggggcgcgcggcgtctgtgcccatcaccttcttg

SEQ ID NO: 143 - DNA, 31 bp, Vibrio Harveyi
atagcatcggttttcgccccgctcttgatgt

SEQ ID NO: 144 - DNA, 44 bp, Bacteroides thetaiotamicron
caacaatcgggcgtacgggtttggggtgcgttcttcccaagaat

SEQ ID NO: 145 - DNA, 24 bp, Bifidobacterium longum
ggaacatcgggggccgcatatccg

SEQ ID NO: 146 - DNA, 34 bp, Treponema denticola
caacaatcttggcttccgcttggcttgtcggccc

SEQ ID NO: 147 - DNA, 47 bp, Bordetella phage
cgttcttcggggcgcgcggcgtctgtgaccacctgattcttgagtag

SEQ ID NO: 148 - DNA, 35 bp, Vibrio Harveyi
gtagcatcggttttcgccccgctttctttgtgtaa

SEQ ID NO: 149 - DNA, 46 bp, Bacteroides thetaiotamicron
ctactctcgggcgtgcgggtttgggttgcgttcttcccaaagatag

SEQ ID NO: 150 - DNA, 47 bp, Bifidobacterium longum
ggcacctcgggggccgcctttctgcgctcggtcgcacgaaggcgtag

SEQ ID NO: 151 - DNA, 37 bp, Treponema denticola
cagcgatcttggcttccgcctggcttgccggccttaa

SEQ ID NO: 152 - DNA, 55 bp, Artificial Sequence
  Synthetic construct
cgctgctgcgctattcggcgtcgacgttttcttgggtctaccgtttaatgtcgac

SEQ ID NO: 153 - DNA, 59 bp, Artificial Sequence
  Synthetic construct
cgctgctgcgctattcggcgtcgacgttttcttgggtctaccgtttaatgtcgaagctt

SEQ ID NO: 154 - DNA, 60 bp, Artificial Sequence
  Synthetic construct
cgctgctgcgctattcggcgtcggcgttttcttgggtctaccgtttaatgtcggaagctt

SEQ ID NO: 155 - DNA, 59 bp, Artificial Sequence; identical to SEQ ID NO: 153
  Synthetic construct

SEQ ID NO: 156 - DNA, 59 bp, Artificial Sequence; identical to SEQ ID NO: 153
  Synthetic construct

SEQ ID NO: 157 - DNA, 59 bp, Artificial Sequence; identical to SEQ ID NO: 153
  Synthetic construct

SEQ ID NO: 158 - DNA, 59 bp, Artificial Sequence
  Synthetic construct
cgctgctgcgctattcggcgtcgccgttttcttgggtctaccgtttaatgtcgaagctt

SEQ ID NO: 159 - DNA, 148 bp, Artificial Sequence
  Synthetic construct
gacgttttcttgggtctaccgtttaatgtcgacggcaactggaacaacacgtcgaactcgggttctcgcgctgcgaactggaacaacgggccgtcgaactcgaacgcgaacatcggggcgcgcggcgtctgtgcccatcaccttcttg

SEQ ID NO: 160 - DNA, 152 bp, Artificial Sequence
  Synthetic construct
tctagacgttttcttgggtctaccgtttaatgtcgacggctactggaacgacacgtcgtactcgggtcctcgcgctgcgtactggaactacgggccgtcgaactcggacgcgagcttcggggcgcgcggcgtctgtgaccacctgattcttg

SEQ ID NO: 161 - DNA, 152 bp, Artificial Sequence
  Synthetic construct
tctagacgttttcttgggtctaccgtttaatgtcgacggccactggtacaacgcgtcgaactcgggttctcgcgctgcgtcctggaactacgggccgtcgtactcgaacgcgagcatcggggcgcgcggcgtctgtgaccacctgattcttg

SEQ ID NO: 162 - DNA, 153 bp, Artificial Sequence
  Synthetic construct
tctagacggttttcttgggtctaccgtttaatgtcgacggcgcctggcacaacccgtcgtactcgggttctcgcgctgcgtcctggaacaacgggccgtcgacctcgagcgcggacatcggggcgcgcggcgtctgtgaccacctgattcttg

SEQ ID NO: 163 - DNA, 152 bp, Artificial Sequence
  Synthetic construct
tctagacgttttcttgggtctaccgtttaatgtcgacggcaactggtgcaacacgtcgtcctcgggttctcgcgctgcgtcctggagctacgggccgtcgaactcgtacgcgggcttcggggcgcgcggcgtctgtgaccacctgattcttg

SEQ ID NO: 164 - DNA, 152 bp, Artificial Sequence
  Synthetic construct
tctagacgttttcttgggtctaccgtttaatgtcgacggcaactggggcatcacgtcgtactcgggttctcgcgctgcgttctggtacagcgggccgtcgttctcgttcgcgttcttcggggcgcgcggcgtctgtgaccacctgattcttg

SEQ ID NO: 165 - DNA, 151 bp, Artificial Sequence
  Synthetic construct
tctagacgttttcttgggctaccgtttaatgtcgacggctactgggtcaacacgtcgaactcgggttctcgcgctgcgtcctggaacaacgggccgtcgaactcgaacgcgagcatcggggcgcgcggcgtctgtgaccacctgattcttg

SEQ ID NO: 166 - DNA, 83 bp, Artificial Sequence
  Synthetic construct
cgctgctgcgctattcggcggcaactggaacaacacgtcgaactcgggtcgacgttttcttgggtctaccgtttaatgtcgac

SEQ ID NO: 167 - DNA, 88 bp, Artificial Sequence
  Synthetic construct
cgctgctgcgctattcggcggcacctgggccaacacgtcgtactcgggtcgacgttttcttgggtctaaccgtttaatgtcgaagctt

SEQ ID NO: 168 - DNA, 87 bp, Artificial Sequence
  Synthetic construct
cgctgctgcgctattcggcggctcctggaacaacacgtcgaactcgggtcgacgttttcttgggtctaccgtttaatgtcgaagctt

SEQ ID NO: 169 - DNA, 87 bp, Artificial Sequence
  Synthetic construct
cgctgctgcgctattcggcggctactggaacagcacgtcgtcctcgggtcgacgttttcttgggtctaccgtttaatgtcgaagctt

SEQ ID NO: 170 - DNA, 87 bp, Artificial Sequence
  Synthetic construct
cgctgctgcgcttttcggcggcgcctggaacggcacgtcggactcgggtcgacgttttcttgggtctaccgtttaatgtcgaagctt

SEQ ID NO: 171 - DNA, 87 bp, Artificial Sequence
  Synthetic construct
cgctgctgcgctattcggcggcgcctggaacagcacgtcggcctcgggtcgacgttttcttgggtctaccgtttaatgtcgaagctt

SEQ ID NO: 172 - DNA, 87 bp, Artificial Sequence
  Synthetic construct
cgctgctgcgctattcggcggcgcctggaacagcgcgtcgaactcgggtcgacgttttcttgggtctaccgtttaatgtcgaagctt

SEQ ID NO: 173 - DNA, 120 bp, Artificial Sequence
  Synthetic construct
gacgttttcttgggtctaccgtttaatgtcgacgttctcgcgctgcgaactggaacaacgggccgtcgaactcgaacgcgaacatcggggcgcgcggcgtctgtgcccatcaccttcttg

SEQ ID NO: 174 - DNA, 124 bp, Artificial Sequence
  Synthetic construct
tctagacgttttcttgggtctaccgtttaatgtcgacgttctcgcgctgcgagctggaactacgggccgtcgagctcgtccgcgaacatcggggcgcgcggcgtctgtgaccacctgattcttg

SEQ ID NO: 175 - DNA, 124 bp, Artificial Sequence
  Synthetic construct
tctagacgttttcttgggtctaccgtttaatgtcgacgttctcgcgctgcgcactggaacaacgggccgtcgaactcgaacgcgttcatcggggcgcgcggcgtctgtgaccacctgattcttg

SEQ ID NO: 176 - DNA, 124 bp, Artificial Sequence
  Synthetic construct
tctagacgttttcttgggtctaccgtttaatgtcgacgttctcgcgctgcgagctggagccacgggccgtcgtactcgagcgcgaacatcggggcgcgcggcgtctgtgaccacctgattcttg

SEQ ID NO: 177 - DNA, 124 bp, Artificial Sequence
  Synthetic construct
tctagacgttttcttgggtctaccgtttaatgtcggcgttctcgcgctgcgaactggaacgacgggccgtcgacctcgagcgcgtacatcggggcgcgcggcgtctgtgaccacctgattcttg

SEQ ID NO: 178 - DNA, 124 bp, Artificial Sequence
  Synthetic construct
tctagacgttttcttgggtctaccgtttaatgtcgacgttctcgcgctgcgtactggaccagcgggccgtcgttctcgttcgcgttcttcggggcgcgcggcgtctgtgaccacctgattcttg

SEQ ID NO: 179 - DNA, 124 bp, Artificial Sequence
  Synthetic construct
tctagacgttttcttgggtctaccgtttaatgtcgacgttctcgcgctgcgagctggcacaacgggccgtcggactcgctcgcgaacatcggggcgcgcggcgtctgtgaccacctgattcttg

SEQ ID NO: 180 - DNA, 120 bp, Artificial Sequence
  Synthetic construct
cgctgctgcgctattcggcggcaactggaacaacacgtcgaactcgggttctcgcgctgcgaactggaacaacgggccgtcgaagtcgacgttttcttgggtctaccgtttaatgtcgac

SEQ ID NO: 181 - DNA, 124 bp, Artificial Sequence
  Synthetic construct
cgctgctgcgctattcggcggcgcctggaacggcacgtcgctctcgggttctcgcgctgcgctctggtacagcgggccgtcggagtcgacgttttcttgggtctaccgtttaatgtcgaagctt

SEQ ID NO: 182 - DNA, 124 bp, Artificial Sequence
  Synthetic construct
cgctgctgcgcgattcggcggcgcctggagcttcacgtcgagctcgggttctcgcgctgcggcctggaacaacgggccgtcgaagtcgacgttttcttgggtctaccgtttaatgtcgaagctt

SEQ ID NO: 183 - DNA, 124 bp, Artificial Sequence
  Synthetic construct
cgctgctgcgctattcggcggcgcctggaacagcgcgtcgaactcgggttctcgcgctgcgtactggaacagcgggccgtcgaagtcgccgttttcttgggtctaccgtttaatgtcgaagctt

SEQ ID NO: 184 - DNA, 124 bp, Artificial Sequence
  Synthetic construct
cgctgctgcgctattcggcggcnactggaacaacacgtcgaactcgggttctcgcgctgcggcctggaacagcgggccgtcgaagtcgacgttttcttgggtctaccgtttaatgtcgaagctt

SEQ ID NO: 185 - DNA, 124 bp, Artificial Sequence
  Synthetic construct
cgctgctgcgctattcggcggctactggaacagcgcgtcgaactcgggttctcgcgctgcgtactggtactacgggccgtcgaagtcgccgttttcttgggtctaccgtttaatgtcgaagctt

SEQ ID NO: 186 - DNA, 83 bp, Artificial Sequence
  Synthetic construct
gacgttttcttgggtctaccgtttaatgtcgacctcgaacgcgaacatcggggcgcgcggcgtctgtgcccatcaccttcttg

SEQ ID NO: 187 - DNA, 86 bp, Artificial Sequence
  Synthetic construct
ttagacgttttcttgggtctaccgtttaatgtcgacctcgctcgcgtacatcggggcgcgcggcgtctgtgaccacctgattcttg

SEQ ID NO: 188 - DNA, 87 bp, Artificial Sequence
  Synthetic construct
tctagacgttttcttgggtctaccgtttaatgtcgacctcgaacgcgaacatcggggcgcgcggcgtctgtgaccacctgattcttg

SEQ ID NO: 189 - DNA, 87 bp, Artificial Sequence
  Synthetic construct
tctagacgttttcttgggtctaccgtttaatgtcgccctcgaccgcgaacatcggggcgcgcggcgtctgtgaccacctgattcttg

SEQ ID NO: 190 - DNA, 88 bp, Artificial Sequence
  Synthetic construct
tctagaccgttttcttgggtctaccgtttaatgtcgccctcgagcgcgtccatcggggcgcgcggcgtctgtgaccacctgattcttg

SEQ ID NO: 191 - DNA, 87 bp, Artificial Sequence
  Synthetic construct
tctagacgttttcttgggtctaccgtttaatgtcgacctcgaacgcgagcatcggggcgcgcggcgtctgtgaccacctgattcttg

SEQ ID NO: 192 - DNA, 36 bp, Artificial Sequence
  Synthetic construct
cgacgttttcttgggtctaccgtttaatgtcgacct

SEQ ID NO: 193 - DNA, 36 bp, Artificial Sequence
  Synthetic construct
ttatctgaacataatgctaccgtttaatgtcgacct

SEQ ID NO: 194 - DNA, 136 bp, Artificial Sequence
  Synthetic construct
cgctgctgcgctattcggcggcaactggaacaacacgtcgaactcgggttctcgcgctgcgaactggaacaacgggccgtcgaagtcgacgttttcttgggtctaccgtttaatgtcgacctcgaacgcgaacatc

SEQ ID NO: 195 - DNA, 135 bp, Artificial Sequence
  Synthetic construct
cgctgctgcgctattcggcggcggctggaacagcacgtcgaactcgggttctcgcgctgcgtcctggagctacgggccgtcgaagtcgacgttttcttgggtcgcccgtttaatgtcgacctcgacgcgaagctt

SEQ ID NO: 196 - DNA, 136 bp, Artificial Sequence
  Synthetic construct
cgctgctgcgctattcggcggcgcctggaactccccgtcggactcgggttctcgcgctgcgtactggtccaacgggccgtcggagtcgacgttttcttgggtctgccgtttaatgtcgacctcgaacgcgaagctt

SEQ ID NO: 197 - DNA, 136 bp, Artificial Sequence
  Synthetic construct
cgctgctgcgctattcggcggcgcctggcgcaacacgtcgtcctcgggttctcgcgctgcgtactggctcgacgggccgtcgaagtcgacgttttcttgggtctcccgtttaatgtcgacctcgaacgcgaagctt

SEQ ID NO: 198 - DNA, 136 bp, Artificial Sequence
  Synthetic construct
cgctgctgcgcgtttcggcggcgcctggaaccacacgtcgaactcgggttctcgcgctgcgagctggaactccgggccgtcgaagtcgacgttttcttgggtcgcccgtttaatgtcgacctcgaacgcgaagctt

SEQ ID NO: 199 - DNA, 136 bp, Artificial Sequence
  Synthetic construct
cgctgctgcgctcttcggcggctactggaacaacccgtcgaactcgggttctcgcgctgcgctctggaacagcgggccgtcgaagtcgacgtgttcttgggtctgccgtttaatgtcgacctcgaacgcgaagctt

SEQ ID NO: 200 - DNA, 136 bp, Artificial Sequence
  Synthetic construct
cgctgctgcgctcttcggcggctactggaacagcgcgtcgaactcgggttctcgcgctgcggactggaactacgggccgtcgctgtcgacgttttcttgggtctaccgtttaatgtcgacctcgaacgcgaagctt

SEQ ID NO: 201 - DNA, 137 bp, Artificial Sequence
  Synthetic construct
cgctgctgcgcttttcggcggctactggctcctcgcgtcggcctcgggttctcgcgctgcgggctggagctccgggccgtcggggtcgccgttttcttgggtcgcccgtttaatgtcgacctcgaacgccgaagctt

SEQ ID NO: 202 - DNA, 136 bp, Artificial Sequence
  Synthetic construct
cgctgctgcgctattcggcggcgcctggaacagcacgtcgaactcgggtgctcgcgctgcggcctggaacagcgggccgtcgaagtcgccgtttccttgggtcgcccgtttaatgtcgacctcgaacgcgaagctt

SEQ ID NO: 203 - DNA, 135 bp, Artificial Sequence
  Synthetic construct
cgctgctgcgctattcggcggcaactggaccaacacgtcgtactcgggttctcgcgctgcgtcctggaactacgggccgtcgaagtcgacgttttcttgggtctgccgtttaatgtcgacctcgaacggaagctt

SEQ ID NO: 204 - DNA, 136 bp, Artificial Sequence
  Synthetic construct
cgctgctgcgctattcggcggcgcctggaactacacgtcgaactcgggttctcgcgctgcgtactggaacttcgggccgtcgaagtcgacgttttcttgggtctaccgtttaatgtcgacctcgaacgcgaagctt

SEQ ID NO: 205 - DNA, 104 bp, Artificial Sequence
  Synthetic construct
gaacaacgggccgtcgaagtcgacgttttcttgggtctaccgtttaatgtcgacctcgaacgcgaacatcggggcgcgcggcgtctgtgcccatcaccttcttg

SEQ ID NO: 206 - DNA, 104 bp, Artificial Sequence
  Synthetic construct
tctagacgggccgtcgaagtcgacgttttcttgggtctcccgttgaatgtcgacctcgtccgcgctcttcggggcgcgcggcgtctgtgaccacctgattcttg

SEQ ID NO: 207 - DNA, 104 bp, Artificial Sequence
  Synthetic construct
tctagacgggccgtcgaagtcgacgttttcttgggtctcccgtttaatgtcgacctcgtacgcgaacctcggggcgcgcggcgtctgtgaccacctgattcttg

SEQ ID NO: 208 - DNA, 104 bp, Artificial Sequence
  Synthetic construct
tctagacgggccgtcgaagtcgacgttttcttgggtctaccgtttaatgtcgccctcgtacgcgggcgtcggggcgcgcggcgtctgtgaccacctgattcttg

SEQ ID NO: 209 - DNA, 104 bp, Artificial Sequence
  Synthetic construct
tctagacgggccgtcgaagtcgacgttttcttgggtctaccgtttaatgtcgacctcgttcgcggacttcggggcgcgcggcgtctgtgaccacctgattcttg

SEQ ID NO: 210 - DNA, 105 bp, Artificial Sequence
  Synthetic construct
tctagacggggccgtcgaagtcgacgttttcttgggtctgccggttaatgtcggcctcgctcgcgagcatcggggcgcgcggcgtctgtggccacctgattcttg

SEQ ID NO: 211 - DNA, 104 bp, Artificial Sequence
  Synthetic construct
tctagacgggccgtcgaagtcgacgttttcttgggtctcccgtttagtgtcgacctcgttcgcgaacgtcggggcgcgcggcgtctgtgaccacctgattcttg

SEQ ID NO: 212 - DNA, 104 bp, Artificial Sequence
  Synthetic construct
tctagacgggccgtcgaagtcgacgttttcttgggtcttccgtttaatgtcgacctcgtccgcgagcctcggggcgcgcggcgtctgtgaccacctgattcttg

SEQ ID NO: 213 - DNA, 104 bp, Artificial Sequence
  Synthetic construct
tctagacgggccgtcgaagtcgacgttttcttgggtctcccgtttaatgtcgacctcgagcgcgtacatcggggcgcgcggcgtctgtgaccacctgattcttg

SEQ ID NO: 214 - DNA, 105 bp, Artificial Sequence
  Synthetic construct
tctagaacgggccgtcgaagtcgacgttttcttgggtctcccgtttaatgtcgccctcgaacgcgtgcatcggggcgcgcggcgtctgtgaccacctgattcttg

SEQ ID NO: 215 - DNA, 104 bp, Artificial Sequence
  Synthetic construct
tctagacgggccgtcgaagtcgacgttttcttgtgtctaccgtttaatgtcgccctcgaacgcgaacatcggggcgcgcggcgtctgtgaccacctgattcttg

SEQ ID NO: 216 - DNA, 99 bp, Artificial Sequence
  Synthetic construct
acgggccgtcgaagtcgacgttttcttgggtctaccgtttaatgtcgacctcgaacgcgaacatcggggcgcgcggcgtctgtgcccatcaccttcttg

SEQ ID NO: 217 - DNA, 104 bp, Artificial Sequence
  Synthetic construct
tctagacgggccgtcgaagtcgacgttttcttgggtctaccgttttatgtcgccctcggacgcgagcatcggggcgcgcggcgtctgtgaccacctgattcttg

SEQ ID NO: 218 - DNA, 106 bp, Artificial Sequence
  Synthetic construct
tctagaccggggccgtcgaagtcgacgttttcttgggtctaccgtttaatgtcgccctcgaacgcgaacatcggggcgcgcggcgtctgtgaccacctgattcttg

SEQ ID NO: 219 - DNA, 104 bp, Artificial Sequence
  Synthetic construct
tctagacgggccgtcgaagtcgacgttttcttgggtctcccgtttaatgtcgacctcgctcgcgctcatcggggcgcgcggcgtctgtgaccacctgattcttg

SEQ ID NO: 220 - DNA, 104 bp, Artificial Sequence
  Synthetic construct
tctagacgggccgtcgaagtcgacgttttcttgggtcgcccgttcaatgtcgccctcgaacgcgaacatcggggcgcgcggcgtctgtgaccacctgattcttg

SEQ ID NO: 221 - DNA, 104 bp, Artificial Sequence
  Synthetic construct
tctagacgggccgtcgaagtcgacgttttcttgggtctgccggttcatgtcgacctcgggcgcgtccttcggggcgcgcggcgtctgtgaccacctgattcttg

SEQ ID NO: 222 - DNA, 104 bp, Artificial Sequence
  Synthetic construct
tctagacgggccgtcgaagtcgacgttttcttgggtctaccgtttaatgtcggcctcgaacgcgatcatcggggcgcgcggcgtctgtgaccacctgattcttg

SEQ ID NO: 223 - DNA, 107 bp, Artificial Sequence
  Synthetic construct
cgctgctgcgctattcggcggcagatctggcgcatccgaatacacggccaacactggcggccgcggatcggtgtacgctctgtctgcgtttgtgttcctgtgctagc

SEQ ID NO: 224 - DNA, 110 bp, Artificial Sequence
  Synthetic construct
cgctgctgcgctattcggcggcggttctggcgcatccgagtccgcggccgccagtggcggccgcggttcggtggacgctctgtctgcgtttgtgttcctgtgctaagctt

SEQ ID NO: 225 - DNA, 111 bp, Artificial Sequence
  Synthetic construct
cgctgctgcgctattcggcggcggatctggcgcctccgtatacccggccagcgctggcgtccgcggatcggtgtccgctctgtctgcgtttgtgttcctgttgctaagctt

SEQ ID NO: 226 - DNA, 110 bp, Artificial Sequence
  Synthetic construct
cgctgctgcgctattcggcggcggctctggcgcctccgaatacacggccaccgctggcggccgcggatcggtgtacgctctgtctgcgtttgtgttcctgtgctaagctt

SEQ ID NO: 227 - DNA, 94 bp, Artificial Sequence
  Synthetic construct
cgctgctgcgctattcggcggcgcctggaacggcacgtcgctctcgggttctcgcgctgcgctctgtctgcgtttgtgttcctgtgctaagctt

SEQ ID NO: 228 - DNA, 94 bp, Artificial Sequence
  Synthetic construct
cgctgctgtgctattcggcggcgcctggaacggcacgtcgctctcgggttctcgcgctgcgctctgtctgcgtttgtgttcctgtgctaagctt

SEQ ID NO: 229 - DNA, 94 bp, Artificial Sequence
  Synthetic construct
cgctgctgcgctattcggcggcgcctggaacggcacgccgctctcgggttctcgcgctgcgctctgtctgcgtttgtgttcctgtgctaagctt

SEQ ID NO: 230 - DNA, 94 bp, Artificial Sequence; identical to SEQ ID NO: 227
  Synthetic construct

SEQ ID NO: 231 - DNA, 94 bp, Artificial Sequence; identical to SEQ ID NO: 227
  Synthetic construct

SEQ ID NO: 232 - DNA, 94 bp, Artificial Sequence; identical to SEQ ID NO: 227
  Synthetic construct

SEQ ID NO: 233 - DNA, 94 bp, Artificial Sequence; identical to SEQ ID NO: 227
  Synthetic construct

SEQ ID NO: 234 - DNA, 94 bp, Artificial Sequence; identical to SEQ ID NO: 227
  Synthetic construct

SEQ ID NO: 235 - DNA, 107 bp, Artificial Sequence; identical to SEQ ID NO: 223
  Synthetic construct

SEQ ID NO: 236 - DNA, 110 bp, Artificial Sequence
  Synthetic construct
gtggggtaacgagttcggcggcgtgaatggcgcatccgaatacacggccctcactggcggccgcggatcggtgtacgctctgtctgcgtttgtgttcctgtgctaagctt

SEQ ID NO: 237 - DNA, 110 bp, Artificial Sequence
  Synthetic construct
gtggggtaacgagttcggcggcgtgaatggcgcatccgaatacacggcctacactggcggccgcggatcggtgtacgctctgtctgcgtttgtgttcctgtgctaagctt

SEQ ID NO: 238 - DNA, 110 bp, Artificial Sequence
  Synthetic construct
gtggggtaacgagttcggcggcgtgaatggcgcatccggttgcacggccaacactggcggccgcggatcggtgtacgctctgtctgcgtttgtgttcctgtgctaagctt

SEQ ID NO: 239 - DNA, 110 bp, Artificial Sequence
  Synthetic construct
gtggggtaacgagttcggcggcgtgaatggcgcatccgaatacgcggccgacacgggcggccgcggatcggtgtacgctctgtctgcgtttgtgttcctgtgctaagctt

SEQ ID NO: 240 - DNA, 110 bp, Artificial Sequence
  Synthetic construct
gtggggtaacgagttcggcggcgtgaatggcgcatccgaatacacggccaacgctggcggccgcggatcggtgtacgctctgtctgcgtttgtgttcctgtgctaagctt

SEQ ID NO: 241 - DNA, 110 bp, Artificial Sequence
  Synthetic construct
gtggggtaacgagttcggcggcgtgaatggcgcatccgaatacacggccaacactggcggccgcggatcggtgtgcgctctgtctgcgtttgtgttcctgtgctaagctt

SEQ ID NO: 242 - DNA, 110 bp, Artificial Sequence
  Synthetic construct
gtggggtaacgagttcggcggcgtgaatggcgcatccgagtacacggccaacactggcggccgcggatcggtgtacgctctgtctgcgtttgtgttcctgtgctaagctt

SEQ ID NO: 243 - DNA, 107 bp, Artificial Sequence; identical to SEQ ID NO: 223
  Synthetic construct

SEQ ID NO: 244 - DNA, 111 bp, Artificial Sequence
  Synthetic construct
gtggggtaacgagttcggcggcggatctggcgcatccgctgacgcggccaacactggcggccgcggatcggtgtacgctctgtctgcgtttgtgttcctgttgctaagctt

SEQ ID NO: 245 - DNA, 110 bp, Artificial Sequence
  Synthetic construct
gtggggtaacgagttcggcggcggctctggcgcatccgcttgcgcggccagcactggcggccgcggctcggtgtacgctctgtctgcgtttgtgttcctgtgctaagctt

SEQ ID NO: 246 - DNA, 110 bp, Artificial Sequence
  Synthetic construct
gtggggtaacgagttcggcggcagatctggcgcatccggttacacggccaaccctggcggccgcggatcggtgtacgctctgtctgcgtttgtgttcctgtgctaagctt

SEQ ID NO: 247 - DNA, 109 bp, Artificial Sequence
  Synthetic construct
gtggggtaacgagttcggcggcagatctggcgcatccgaatacacggccaaccctggcggccgcggttcggtgtgcgctctgtctgcgtttgtgttctgtgctaagctt

SEQ ID NO: 248 - DNA, 110 bp, Artificial Sequence
  Synthetic construct
gtggggtaacgagttcggcggcagatctggcgcatccgctcacacggccaacactggcggccgcggatcgctgtacgctctgtctgcgtttgtgttcctgtgctaagctt

SEQ ID NO: 249 - DNA, 79 bp, Artificial Sequence
  Synthetic construct
tctgtctgcgtttgtgttcctgtgctagcctcgaacgcgaacatcggggcgcgcggcgtctgtgcccatcaccttcttg

SEQ ID NO: 250 - DNA, 82 bp, Artificial Sequence
  Synthetic construct
agatctgtctgcgtttgtgttcctgtgctagcctcgtacgcgaccttcggggcgcgcggcgtctgtgaccacctgattcttg

SEQ ID NO: 251 - DNA, 82 bp, Artificial Sequence
  Synthetic construct
agatctgtctgcgtttgtgttcctgtgctagcctcgtacgcgaacatcggggcgcgcggcgtctgtgaccacctgattcttg

SEQ ID NO: 252 - DNA, 82 bp, Artificial Sequence
  Synthetic construct
agatctgtctgcgtttgtgttcctgggctggcctcgagcgcgaacatcggggcgcgcggcgtctgtgaccacctgattcttg

SEQ ID NO: 253 - DNA, 82 bp, Artificial Sequence
  Synthetic construct
agatctgtctgcgtttgtgttcctgtgctagcctcgaacgcgaacctcggggcgcgcggcgtctgtgaccacctgattcttg

SEQ ID NO: 254 - DNA, 82 bp, Artificial Sequence
  Synthetic construct
agatctgtctgcgtttgtgttcctgtgctagcctcgagcgcgaacatcggggcgcgcggcgtctgtgaccacctgattcttg

SEQ ID NO: 255 - DNA, 82 bp, Artificial Sequence
  Synthetic construct
agatctgtctgcgtttgtgttcctgtgctagcctcgaccgcgagcatcggggcgcgcggcgtctgtgaccacctgattcttg

SEQ ID NO: 256 - DNA, 82 bp, Artificial Sequence
  Synthetic construct
agatctgtctgcgtttgtgttcctgtgctagcctcgaacgcgagcatcggggcgcgcggcgtctgtgaccacctgattcttg

SEQ ID NO: 257 - DNA, 82 bp, Artificial Sequence
  Synthetic construct
agatctgtctgcgtttgtgttcctgtgctagcctcgggcgcgagcatcggggcgcgcggcgtctgtgaccacctgattcttg

SEQ ID NO: 258 - DNA, 82 bp, Artificial Sequence
  Synthetic construct
agatctgtctgcgtttgtgttcctgtgctagcctcgggcgcgtacatcggggcgcgcggcgtctgtgaccacctgattcttg

SEQ ID NO: 259 - DNA, 82 bp, Artificial Sequence; identical to SEQ ID NO: 258
  Synthetic construct

SEQ ID NO: 260 - DNA, 82 bp, Artificial Sequence; identical to SEQ ID NO: 256
  Synthetic construct

SEQ ID NO: 261 - DNA, 107 bp, Artificial Sequence; identical to SEQ ID NO: 223
  Synthetic construct

SEQ ID NO: 262 - DNA, 110 bp, Artificial Sequence
  Synthetic construct
gtggggtaacgagttcggcggcgtgaatggcgcatccgaatacacggccaacactggcggccgcggctcggtgtacgctctgtctgcgtttgtgttcctgtgctaagctt

SEQ ID NO: 263 - DNA, 110 bp, Artificial Sequence
  Synthetic construct
gtggggtaacgagttcggcggcgtgaatggcgcgtccgagtacacggccaacaccggcggccgcggatcggtgtacgctctgtctgcgtttgtgttcctgtgctaagctt

SEQ ID NO: 264 - DNA, 110 bp, Artificial Sequence
  Synthetic construct
gtggggtaacgagttcggcggcgtgaatggcgcatccgaatacacggccaacactggcggccgcggatcggtgtacgctctgtctgcgtttgtgttcctgtgctaagctt

SEQ ID NO: 265 - DNA, 110 bp, Artificial Sequence; identical to SEQ ID NO: 264
  Synthetic construct

SEQ ID NO: 266 - DNA, 107 bp, Artificial Sequence; identical to SEQ ID NO: 223
  Synthetic construct

SEQ ID NO: 267 - DNA, 110 bp, Artificial Sequence
  Synthetic construct
gtggggtaacgagttcggcggcgggtctggcgcgtccggctgcacggccaacactggcggccgcggctcggtgtacgctctgtctgcgtttgtgttcctgtgctaagctt

SEQ ID NO: 268 - DNA, 110 bp, Artificial Sequence
  Synthetic construct
gtggggtaacgagttcggcggccgatctggcgcatccggatacccggccagccctggcggccgcggatcggtgtacgctctgtctgcgtttgtgttcctgtgctaagctt

SEQ ID NO: 269 - DNA, 111 bp, Artificial Sequence
  Synthetic construct
gtggggtaacgagttcggcggcggatctggcgcatccgaatacgcggccaacactggcggccgcggatcggtgttcgctctgtctgcgtttgtgttcctgtgcctaagctt

SEQ ID NO: 270 - DNA, 110 bp, Artificial Sequence
  Synthetic construct
gtggggtaacgagttcggcggcgggtctggcgcttccgagtacacggcctacactggcggccgcggatcggtgtacgctctgtctgcgtttgtgttcctgtgctaagctt

SEQ ID NO: 271 - DNA, 110 bp, Artificial Sequence
  Synthetic construct
gtggggtaacgagttcggcggcggacctggcgcctccggatacacggcctacactggcggccgcggatcggtggacgctctgtctgcgtttgtgttcctgtgctaagctt

SEQ ID NO: 272 - DNA, 110 bp, Artificial Sequence
  Synthetic construct
gtggggtaacgagttcggcggcagatctggcgcatccggctacacggccaacgctggcggccgcggatcggtgttcgctctgtctgcgtttgtgttcctgtgctaagctt

SEQ ID NO: 273 - DNA, 110 bp, Artificial Sequence
  Synthetic construct
gtggggtaacgagttcggcggcggttctggcgcatccgagtacccggccaacactggcggccgcggatcggtgtacgctctgtctgcgtttgtgttcctgtgctaagctt

SEQ ID NO: 274 - DNA, 110 bp, Artificial Sequence
  Synthetic construct
gtggggtaacgagttcggcggcagatctggcgcatccgggtacacggccaacactggcggccgcggatcggtgtgcgctctgtctgcgtttgtgttcctgtgctaagctt

SEQ ID NO: 275 - DNA, 110 bp, Artificial Sequence
  Synthetic construct
gtggggtaacgagttcggcggcagatctggcgcatccgaatacacggccaacactggcggccgcggatcggtgtacgctctgtctgcgtttgtgttcctgtgctaagctt

SEQ ID NO: 276 - DNA, 110 bp, Artificial Sequence
  Synthetic construct
gtggggtaacgagttcggcggcagatctggcgcatccgaatacacggccaacactggcggccgcggatcggtgtacgctctggctgcgtttgtgttcctgtgctaagctt

SEQ ID NO: 277 - DNA, 110 bp, Artificial Sequence; identical to SEQ ID NO: 275
  Synthetic construct

SEQ ID NO: 278 - DNA, 79 bp, Artificial Sequence; identical to SEQ ID NO: 249
  Synthetic construct

SEQ ID NO: 279 - DNA, 82 bp, Artificial Sequence
  Synthetic construct
agatctgtctgcgtttgtgttcctgtgctagcctcgtacgcggacctcggggcgcgcggcgtctgtgaccacctgattcttg

SEQ ID NO: 280 - DNA, 82 bp, Artificial Sequence; identical to SEQ ID NO: 251
  Synthetic construct

SEQ ID NO: 281 - DNA, 82 bp, Artificial Sequence
  Synthetic construct
agatctgtctgcgtttgtgttcctgtgctagcctcgaccgcgtgcatcggggcgcgcggcgtctgtgaccacctgattcttg

SEQ ID NO: 282 - DNA, 82 bp, Artificial Sequence; identical to SEQ ID NO: 254
  Synthetic construct

SEQ ID NO: 283 - DNA, 82 bp, Artificial Sequence
  Synthetic construct
agatctgtctgcgtttgtgttcctgtgctagcctcgtccgcgggcatcggggcgcgcggcgtctgtgaccacctgattcttg

SEQ ID NO: 284 - DNA, 82 bp, Artificial Sequence
  Synthetic construct
agatctgtctgcgtttgtgttcctgtcctagcctcggccgcgtacatcggggcgcgcggcgtctgtgaccacctgattcttg

SEQ ID NO: 285 - DNA, 82 bp, Artificial Sequence
  Synthetic construct
agatctgtctgcgtttgtgttcctgtgctagcctcgancgcgaacatcggggcgcgcggcgtctgtgaccacctgattcttg

SEQ ID NO: 286 - DNA, 82 bp, Artificial Sequence
  Synthetic construct
agatctgtctgcgtttgtgttcctgtgctagcctcgtacgcgcacatcggggcgcgcggcgtctgtgaccacctgattcttg

SEQ ID NO: 287 - DNA, 82 bp, Artificial Sequence
  Synthetic construct
agatctgtctgcgtttgtgttcctgtgctagcctcgaacgcgtccttcggggcgcgcggcgtctgtgaccacctgattcttg

SEQ ID NO: 288 - DNA, 82 bp, Artificial Sequence
  Synthetic construct
agatctgtctgcgtttgtgttcctgtgctagcctcttacgcgaacgtcggggcgcgcggcgtctgtgaccacctgattcttg

SEQ ID NO: 289 - DNA, 82 bp, Artificial Sequence
  Synthetic construct
agatctgtctgcgtttgtgtttctgtgctagcctcgagcgcgaactttggggcgcgcggcgtctgtgaccacctgattcttg

(19) United States
(12) Patent Application Publication
(10) Pub. No.: US 2010/0041033 A1
(43) Pub. Date: Feb. 18, 2010

(54) SITE SPECIFIC SYSTEM FOR GENERATING DIVERSITY PROTEIN SEQUENCES

(75) Inventors: Jeffery F. Miller, Santa Monica, CA (US); Sergei Doulatov, Toronto (CA); Asher Hodes, Los Angeles, CA (US); Min Rennella, Los Angeles, CA (US); Mari Gingery, Glendale, CA (US); Huatao Guo, Encino, CA (US); David W. Martin, Mill Valley, CA (US)

Correspondence Address: DLA PIPER LLP (US), 4365 EXECUTIVE DRIVE, SUITE 1100, SAN DIEGO, CA, 92121-2133, US

(73) Assignees: The Regents of the University of California, Oakland, CA (US); AvidBiotics Corp., South San Francisco, CA (US)

(21) Appl. No.: 12/368,949

(22) Filed: Feb. 10, 2009

Related U.S. Application Data
(63) Continuation-in-part of application No. 11/197,219, filed on Aug. 3, 2005, now Pat. No. 7,585,957.
(60) Provisional application No. 60/598,617, filed on Aug. 3, 2004.

(51) Int. Cl. C12Q 1/68; C12N 15/31; C12N 1/21; C12N 15/74; C40B 40/06

(52) U.S. Cl. 435/6; 536/23.1; 435/252.3; 435/471; 506/16

(57) ABSTRACT

This invention relates to the diversification of nucleic acid sequences by use of a nucleic acid molecule containing a region of sequence that acts as a template for diversification. The invention thus provides nucleic acid molecules to be diversified, as well as those which act as the template region (TR) and in concert with the TR for directional, site-specific diversification. Further provided are methods of preparing and using these nucleic acid sequences.

### RELATED APPLICATIONS

This application is a continuation-in-part application of U.S. application Ser. No. 11/197,219, titled: “SITE SPECIFIC SYSTEM FOR GENERATING DIVERSITY PROTEIN SEQUENCES,” filed Aug. 3, 2005, which claims benefit of priority from U.S. Provisional Patent Application Ser. No. 60/598,617, filed Aug. 3, 2004, each of which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with U.S. Government support of Grant Nos. RO1 AI38417, AI061598 and A1071204, awarded by the NIH and 1999-02298, awarded by the USDA. The U.S. Government has certain rights in this invention.

### FIELD OF THE INVENTION

This invention relates to the diversification of nucleic acid sequences by use of a nucleic acid molecule containing a region of sequence that acts as a template for diversification. The invention thus provides nucleic acid molecules to be diversified, those which act as the template region (TR) for directional, site-specific diversification and for encoding necessary enzymes, and methods of preparing, as well as using them.

### BACKGROUND OF THE INVENTION

Bordetella bacteriophages generate diversity in a gene that specifies host tropism for the host bacterium. This adaptation is produced by a genetic element that combines transcription, reverse transcription and integration with site-directed, adenine-specific mutagenesis. Necessary to this process is a reverse transcriptase-mediated exchange of information between two regions, one serving as a donor template region (TR) and the other as a recipient of variable sequence information, the variable region (VR).

Bordetella species that cause respiratory infections in mammals, including humans, serve as hosts for a family of bacteriophages that encode a diversity-generating system which allows the bacteriophage to use different receptor molecules on the bacteria for attachment and subsequent infection (Liu, M. et al. Reverse transcriptase-mediated tropism switching in Bordetella bacteriophage. Science 295, 2091-2094 (2002) and Liu, M. et al. Genomic and genetic analysis of Bordetella bacteriophages encoding reverse transcriptase-mediated tropism-switching cassettes. J. Bacteriol. 186, 1503-17 (2004)). The Bordetella cell surface is highly variable as a result of a complex program of gene expression mediated by the BvgAS phosphorelay, which regulates the organism's infectious cycle (Ackerley, B. J., Cotter P. A., & Miller, J. F. Ectopic expression of the flagellar regulon alters development of the Bordetella-host interaction. Cell 80, 611-620 (1995); Uhl, M. A. & Miller, J. F. Integration of multiple domains in a two-component sensor protein: the Bordetella pertussis BvgAS phosphorelay. EMBO J. 15, 1028-1036 (1996); Cotter, P. A. & Miller, J. F. Bordetella. In Principles of Bacterial Pathogenesis. E. Groisman, Ed. Academic Press, San Diego, Calif. pp. 619-674 (2000); and Mattoo, S., Foreman-Wykert, A. K., Cotter, P. A., Miller, J. F. Mechanisms of Bordetella pathogenesis. Front Biosci 6, E168-E186 (2001)).

Bacteriophage (“phage”) BPP-1 preferentially infects virulent, Bvg+ Bordetella bacteria due to differential expression of phage receptor, pertactin (Prn), on the bacterial outer membrane (see FIG. 1a herein and Emsley, P., Charles, I. G., Fairweather, N. F., Isaacs, N. W. Structure of the Bordetella pertussis virulence factor P.69 pertactin. Nature 381, 90-92 (1996); van den Berg, B. M., Beekhuizen, H., Willems, R. J., Mooi, F. R., van Furth, R. Role of Bordetella pertussis virulence factors in adherence to epithelial cell lines derived from the human respiratory tract. Infect Immun 67, 1056-1062 (1999); and King, A. J. et al. Role of the polymorphic region 1 of the Bordetella pertussis protein pertactin in immunity. Microbiology 147, 2885-2895 (2001)). At characteristic frequencies, BPP-1 gives rise to tropic variants (BMP and BIP) that recognize distinct surface receptors and preferentially infect avirulent, Bvg− bacteria or are indiscriminate to the Bvg status, respectively. These viral parasites have thus evolved to keep pace with the dynamic surface structure displayed by their target host as it traverses its infectious cycle.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The invention is based in part on the discovery that the agile tropism switching, that is switching the ability to infect specific bacteria, in Bordetella bacteriophages is mediated by a variability-generating cassette encoded in the phage genome (see FIG. 1b herein). This cassette functions to introduce nucleotide substitutions at 23 sites in a 134 bp variable region (VR) present at the 3′ end of the mtd locus. Mtd, a putative tail protein, is necessary for phage morphogenesis and infectivity, and the sequence of VR within Mtd determines tropism (bacterial host) specificity. Binding of a BPP-1 derived GST-Mtd fusion protein to the Bordetella cell surface is dependent on expression of protein pertactin (Prn) on the outer membrane of the bacteria, correlating with the infective properties of the parental phage. The cassette shown in FIG. 1b therefore functions to generate plasticity in a ligand-receptor interaction via site-directed mutagenesis of, and diversification within, VR sequences.

Thus in a first aspect, the invention provides for a nucleic acid molecule comprising a variable region (VR) which is operably linked to a template region (TR) wherein said TR is a template sequence that directs site-specific mutagenesis of said VR. The nucleic acid molecule may be recombinant, in the sense that it comprises nucleic acid sequences that are not found together in nature, such as sequences that are synthetic (non-naturally occurring) and/or brought together by use of molecular biology and genetic engineering techniques from heterologous sources. Alternatively, the nucleic acid molecule may be isolated, in the sense that it comprises naturally occurring sequences isolated from the surrounding biological factors or sequences with which they are found in nature.

An operable linkage between the VR and TR regions of a nucleic acid molecule of the invention refers to the ability of the TR to serve as the template for directional, site-specific mutagenesis or diversification of the sequence in the VR. Thus in one possible embodiment of the invention, a recombinant nucleic acid molecule may comprise a donor template region (TR) and a variable region (VR) that are physically attached in cis such that the TR serves as the template sequence to direct site-specific mutagenesis in the VR. The separation between the TR and VR regions may be of any distance so long as they remain operably linked. In another embodiment, the TR and VR may not be linked in cis, but the TR retains the ability to direct site specific mutagenesis of the VR. Thus the TR and VR regions may be operably linked in trans, such that the sequences of each region are present on separate nucleic acid molecules.

The invention thus also provides for a pair of nucleic acid molecules wherein a first molecule of the pair comprises a VR which is operably linked to a TR on a second molecule of the pair. As provided by the invention, the TR is a template sequence that directs site-specific mutagenesis of said VR. The nucleic acid molecules are optionally recombinant, in the sense that they may comprise nucleic acid sequences that are not found together in nature, such as sequences that are brought together by use of molecular biology and genetic engineering techniques from heterologous sources. Of course, sequences that are brought together may be synthetic (non-naturally occurring) sequences or those that are from naturally occurring sequences but isolated from the surrounding biological factors or sequences with which they are found in nature.

In embodiments of the invention wherein the VR and TR are in trans, the TR is operably linked to sequences encoding a reverse transcriptase (RT) activity as described below. As such, the VR and reverse transcriptase encoding sequence(s) are also present in trans to each other. In some embodiments, the TR and RT activity coding sequence are in cis to each other, optionally with the TR and RT coding sequence originating from the same organism. In other embodiments, the TR and RT coding sequence may be in trans to each other while remaining operably linked so that the TR still directs RT mediated changes in the operably linked VR. Of course, the TR and/or RT coding sequence may be altered as described below relative to the naturally occurring TR in the organism. Alternatively, the TR and RT coding sequence may be heterologous to each other in that they originate from, or are isolated from, different organisms, or one or the other or both are synthetic (non-naturally occurring) or synthesized (rather than isolated). Synthetic sequences include those which are derived from naturally occurring sequences.

The invention is also based in part on the discovery that sites of variability in the VR of Bordetella bacteriophages correspond to adenine residues in the generally homologous template region, TR, which itself is invariant and essential for tropism switching. The invention is also based in part on the discovery that (translationally) silent (or “synonymous”) substitutions in TR are transmitted to VR during switching, with TR supplying the raw sequence information for variability.

Thus the recombinant nucleic acid molecules of the invention include initial molecules wherein the TR region is identical to the VR, such that the adenine residues present in the TR will result in the mutagenesis or diversification of the corresponding positions in the VR sequence. Stated differently, the invention provides a recombinant nucleic acid molecule wherein the sequence of said TR is a perfect direct repeat of the sequence in said VR such that upon diversification of the VR region, one or more adenine residues in the VR, also found in the TR, will be mutated to another nucleotide, that is cytosine, thymine or guanine, without change in the TR sequence.

Alternatively, the invention provides recombinant nucleic acid molecules wherein the TR and VR regions are not identical such that as the TR region directs diversification of the VR. Such diversification may include the mutagenesis of nucleotide residues in the VR based upon the presence of corresponding adenine residues in the TR.

Without being bound by theory, and offered to improve the understanding of the invention, this ability is shown herein to be mediated by an RNA intermediate generated by a reverse transcription based mechanism in which a TR RNA transcript serves as a template for reverse transcription during which the nucleotides incorporated opposite the adenine residues of the TR RNA transcript are randomized in the resulting single-stranded cDNA. The TR-derived, mutagenized cDNA sequence is then used to replace all or part of the VR in a process termed “mutagenic homing.” Support for this mechanism is provided by the discovery that in Bordetella bacteriophages, the brt locus, which encodes a reverse transcriptase (RT), is essential for the generation of diversity. Additional support is provided by the discovery that mutagenesis occurs exclusively at sites occupied by adenines in the TR. Artificial substitution of an adenine in the TR with another nucleotide subsequently abolishes variation at that corresponding position in the VR, while introduction of an ectopic adenine subsequently produces a novel site of heterogeneity in the VR.

Thus in a further aspect, the invention provides for the diversification of VR sequences via the presence of adenine residues in the TR operably linked to the VR. The invention provides for a nucleic acid molecule wherein the TR region contains one or more adenine residues not found in the VR, such that the adenine residues present in the TR will result in the mutagenesis or diversification of the corresponding positions in the VR sequence. Stated differently, the invention provides a recombinant nucleic acid molecule wherein the sequence of said TR is an imperfect direct repeat of the sequence in said VR due to the substitution of one or more adenine residues for one or more non-adenine residues in said VR. This may be referred to as adenine-mediated diversification.

Alternatively, as compared to the VR, the TR contains one or more insertions of adenine, optionally with the insertion of additional nucleotides to maintain the correct reading frame. As a non-limiting example, groups of three nucleotides (including one or more adenines) may be inserted in-frame into the TR in order to direct the insertion of a variable codon into the VR.

In other embodiments, the invention provides for the diversification of VR sequences via the alternation of other of nucleotide residues in the TR operably linked to the VR. As a non-limiting example, the invention provides a TR that contains a deletion of one or more codons is used to direct the deletion of corresponding codons from the operably linked VR. As another example, the TR contains an insertion of one or more codons to direct the insertion of the inserted codon(s) into the operably linked VR. The TRs of the invention also include those where the TR contains a deletion or insertion of one or more nucleotides, relative to the operably linked VR, to alter the reading frame of the VR. The deletion or insertion of nucleotides in a TR to direct deletions or insertions in an operably linked VR may be used simultaneously, such as where one portion of the TR is used to direct deletion of nucleotides while another portion of the TR is used to direct insertion of nucleotides. This may be referred to as deletion/insertion mediated diversification.

In yet additional embodiments, the invention provides for diversification based upon non-adenine substitutions of residues in the TR. Thus a nucleotide in the TR may be substituted with a non-adenine residue such that the substitution is transferred to the corresponding position in the operably linked VR. As a non-limiting example, a cytosine (C) to guanine (G) substitution in a TR can be used to result in the same C to G substitution in the operably linked VR. This may be referred to as substitution-mediated diversification.

The invention also provides for the use of adenine-mediated, deletion/insertion mediated, and/or substitution-mediated diversification in any combination to alter the sequence of a VR.

In some nucleic acid molecules of the invention, an RT encoding region, and/or an atd region (or bbp7 region), in the vicinity of the 5′ end of a TR may also be present. These regions may be present in cis relative to the TR region. Thus in embodiments of the invention wherein the VR and TR are in trans to each other, the atd region may be in trans relative to the VR. In other embodiments, the atd region is absent or substituted by a functionally analogous region of sequence, such as a promoter sequence that regulates or directs the expression of the TR region and operably linked RT encoding sequence.

As explained above, one property of the diversity-generating system of the invention is the directional transfer of sequence information which accompanies mutagenesis. Thus one TR is able to direct sequence changes in one or more operably linked VRs. Although a VR is highly variable, the operably linked TR is maintained as an uncorrupted source of sequence information including the information to retain the basic structural integrity of the VR encoded protein molecule. The invention is further based on the identification of a nucleic acid sequence designated IMH (initiation of mutagenic homing), which functions in determining the direction of the TR to VR transfer of sequence information.

In some embodiments of the invention, the IMH sequences are those located at the 3′ end of each region in Bordetella bacteriophages and which comprise a 14 bp segment consisting of G and C residues followed by a 21 bp sequence. The IMH sequences at the 3′ end of the VR differ at 5 positions from the sequences in the corresponding TR region (see FIG. 1c herein). The invention is also based in part on the demonstration that these polymorphisms form part of a cis-acting site that determines the directionality of homing. The demonstration was made by substituting the 21 bp VR IMH sequence with the corresponding IMH-like sequence associated with the 3′ end of the TR (BPP-3′TR). The result was an elimination of tropism switching. The reverse substitution of the corresponding TR IMH-like sequence for the VR IMH sequence (BPP-3′VR) did not affect switching. Instead, the placement of VR IMH sequence at the 3′ ends of both VR and TR resulted, surprisingly, in the generation of adenine-dependent variability in TR as well as in VR (see FIG. 1d herein), an event not previously observed in wild type phage. Variability continued to occur solely at positions occupied by adenine residues in the parental TR, indicating that the basic mechanism of mutagenesis was retained. Furthermore, the pattern of mutations observed in different BPP-3′VR phage indicated that TR was the sole source of both TR and VR variability (see (FIG. 1d herein).

These observations demonstrate that the sequence designated as IMH helps determine the direction of transfer of sequence information from the TR to the VR. They also support the use of the corresponding TR IMH-like sequence at the 3′ end of the TR to prevent corruption of TR while the IMH directs variability to VR. Furthermore, deletion analysis indicated that in VR, the 5′ boundary of information transfer is established by the extent of homology between VR and TR.

The recombinant nucleic acid molecules of the invention may thus contain an IMH sequence located at the 3′ end of the VR and an IMH-like sequence at the end of the TR, Alternatively, the molecules may contain an IMH sequence at the end of both the VR and the TR such that the sequence of the TR may also vary to result in a “super-diversity” generating system.

In embodiments of the invention wherein a sequence of interest (or “desired VR”) to be diversified is not operably linked to the necessary TR region, an IMH sequence can be operably located at the 3′ of the desired VR followed by operable linkage to an appropriate TR with its IMH-like 3′-region. A non-limiting example of such a system is seen in the case of a desired VR which is all or part of a genomic sequence of a cell wherein insertion of an appropriate IMH and introduction of a TR containing construct with the appropriate corresponding IMH-like region, optionally with a cis linked RT coding sequence, is used to diversify the desired VR. The TR may simply be a direct repeat of the desired VR sequence to be diversified or mutagenized via the adenines present in the TR. Alternatively, the TR may contain ectopic adenines, deletions/insertions, and/or substitutions at positions corresponding to those specific sites of VR where diversity is desired. The length of homology between TR and VR can be used to functionally define the desired VR to be diversified.

The desired VR of the invention may be any nucleic acid sequence of interest for mutagenesis or diversification by use of the instant invention. In some embodiments, the sequence is all or part of a sequence encoding a binding partner of a target molecule. Target molecules may be any cellular factor or portion thereof which is of interest to a skilled person practicing the invention. Non-limiting examples include polypeptides, cell surface molecules, carbohydrates, lipids, hormones, growth or differentiation factors, cellular receptors, a ligand of a receptor, bacterial proteins or surface components, cell wall molecules, viral particles, immunity or immune tolerance factors, MHC molecules (such as Class I or II), tumor antigens found in or on tumor cells, and others as desired by a skilled practitioner and/or described herein. The binding partner (encoded at least in part by the desired VR) may be any polypeptide which, upon expression, binds to the target molecule, such as under physiological conditions or laboratory (in vivo, in vitro, or in culture) conditions.

In some embodiments of the invention, the binding partner is a bacteriocin (including a vibriocin, pyocin, or colicin), a bacteriophage protein (including a tail component that determines host specificity), capsid or surface membrane component (including those that determine physiologic, pharmacologic, or pharmaceutical properties), a ligand for a cell surface factor or an identified drug or diagnostic target molecule, or other molecules as desired and/or described herein.

Any portion, or all, of the coding region for a binding partner can be used as the desired VR. In some embodiments of the invention, however, the desired VR is the 3′ portion of said sequence encoding said binding partner. The 3′ portion of a coding sequence ends at the last codon. In other embodiments of the invention, the desired VR is located within about 50, about 100, about 150, about 200, about 250, about 300, or about 350 or more codons of the last codon in a coding sequence to be diversified. Stated differently, the desired VR may contain about 20, about 50, about 100, about 150, about 200, about 250, about 300, about 400, about 450, about 500, about 550, about 600, about 650, about 700, about 750, about 800, about 900, about 950, about 1000, about 1500, about 2000, about 2500, or about 3000 or more nucleotides from the last nucleotide of the coding region. In some embodiments, the IMH is not part of the translated portion of the VR, and as such may optionally be in an intron. Stated differently, some embodiments of the invention provide for an IMH which is transcribed, but not translated, or not transcribed or translated, while the VR and the larger sequence containing the VR may be transcribed and translated and encode a polypeptide.

In additional embodiments, the binding partner may be part of a fusion protein such that it is produced as a chimeric protein comprising another polypeptide. The other polypeptide member of the fusion protein may be heterologous to the binding partner. Alternatively, it may be another portion of the same binding partner such that the fusion protein is a recombinant molecule not found in nature.

In other embodiments, the desired VR for site specific mutagenesis is a non-translated, and optionally non-transcribed, regulatory region. The invention may be utilized to diversify such regulatory sequences to modify their function. In the case of 5′ regulatory elements, as a non-limiting example, the invention may be used to derive regulatory regions that direct expression more strongly (e.g. a stronger promoter) or less strongly (e.g. a weaker promoter). Alternatively, the regulatory regions may be diversified to increase or decrease their sensitivity to regulation (e.g. more tightly or less tightly regulated). In the case of 3′ regulatory elements, the invention may be used to derive regions that increase or decrease the stability of expressed RNA molecules. Other regulatory sequences may be similarly diversified.

As described above, the invention also provides for isolated nucleic acid molecules derived from naturally occurring sequences. Such an isolated nucleic acid molecule may be described as comprising a donor template region (TR) and a variable region (VR) wherein said TR is a template sequence operably linked to said VR to direct site specific mutagenesis of said VR. These isolated nucleic acid molecules may comprise the coding sequence containing the VR and TR as well as other components necessary to direct site specific mutagenesis of the VR in a heterologous system. Non-limiting examples of additional sequences from naturally occurring sequences are those that encode an RT activity and those that function as an IMH, to provide directionality to the transfer of sequence information from a TR to a VR, or an IMH-like sequence to prevent or reduce the frequency of changes in the TR sequence. Molecules containing these VR and TR regions with these other components are termed diversity generating retroelements (DGRs) of the invention.

These isolated nucleic acid molecules may also serve as a source of additional IMH sequences, RT coding regions, and atd regions for use in the practice of the instant invention. Non-limiting examples of isolated nucleic acid molecules include those shown in FIG. 2 herein. These include molecules isolated from Vibrio harveyi ML phage, Bifidobacterium longum, Bacteroides thetaiotaonicron, Treponema denticola, or a DGR from cyanobacteria. Non-limiting examples of such cyanobacteria include Trichodesmium erythraeum #1, Trichodesmium erythraeum #2, Nostoc PPC ssp. 7120 #1, Nostoc PPC ssp. 7120 #2, or Nostoc punctiforme. The relevant sequences illustrated in FIG. 2 are all publicly available and accessible to the skilled person.

In some embodiments, the invention provides an isolated nucleic acid molecule comprising a donor template region (TR) and an operably linked RT coding sequence. Such a molecule is preferably not from Bvg+ tropic phage-1 (BPP-1), Bvg⁻ tropic phage-1 (BMP-1), or Bvg indiscriminate phage-1 (BIP-1) bacteriophage. The isolated molecule may be from a bacteriophage, a prophage of a bacterium, a bacterium, or a spirochete.

Of course, cells comprising the nucleic acid molecules of the invention are also provided. Such cells may be prokaryotic or eukaryotic, and are capable of supporting site-specific mutagenesis as described herein. Cells that are not capable of supporting such mutagenesis may still be used to replicate nucleic acid molecules of the invention or to generate their encoded protein molecules for subsequent use. In the case of eukaryotic cells, the nucleic acids of the invention may be modified for their use in a eukaryotic environment. These modifications include the use of promoter sequences recognized by a eukaryotic RNA polymerase; the introduction of intron sequences in the TR-brt to facilitate export of RNA transcripts from nucleus to cytoplasm for translation of the brt, and the presence of a nuclear localization signal (NLS) coding sequence as part of the RT coding sequence such that the RT polypeptide contains a NLS to direct its transport to, and/or retention in, the eukaryotic nucleus. In some embodiments, the NLS is located at the N or C terminus of the RT polypeptide.

In an additional aspect, the invention provides a method of site-specific mutagenesis of a nucleic acid sequence of interest present as a VR of the invention. Such a method would comprise the use of a nucleic acid molecule as described herein wherein the VR comprises said nucleic acid sequence of interest and the TR is a direct repeat of the VR or the sequence of interest. Thus, mutagenesis will be limited to the adenine residues present in the TR. Alternatively, a non-identical TR, such as a repeat of the VR or the sequence of interest containing ectopic adenine residues, insertions, deletions, or substitutions may be used. The method would further include the expression of such nucleic molecules in a cell such that one or more nucleotide positions of the VR or sequence of interest is substituted by a different residue.

Such methods of the invention may be performed to allow more than one nucleotide position of the VR or the sequence of interest to be substituted. As noted above, the VR or sequence of interest may encode all or part (such as the 3′ portion) of a binding partner of a target molecule. These methods of the invention may, of course, be used to alter the binding properties of a binding partner such that its interaction with a target molecule will be changed. Non-limiting examples of such alternations include changing the specificity or binding affinity of a binding partner. The methods may be used to modify a particular binding partner such that it will bind a different target molecule. A non-limiting example of this aspect of the invention is the modification of a phage tropism determinant such that it will bind a heterologous bacterial surface component of interest. A bacteriophage that is made to express such a derivative would thus be infectious for a heterologous bacterium. This may be advantageously used as a means of creating phage or phage parts capable of binding to, infecting and/or killing (e.g. via lysis or dissipation of membrane potential) a particular strain of bacteria not normally affected by phage expressing the progenitor tropism determinant. The invention may also be used as a means of broadening or expanding the bacteriophage host range, or the binding range of a part or parts thereof, to include target molecules, species, or strains not commonly bound or infected by the parent phage or any phage. Another non-limiting example is modification of a sequence to restore or alter a binding or enzymatic activity, such as restoration of a phosphotransferase activity.

As described herein, site-specific mutagenesis of a known bacteriophage protein also may be practiced by the use of an isolated nucleic acid molecule containing a naturally occurring combination of VR and TR as described herein. Non-limiting examples of such molecules include those from Vibrio harveyi ML phage, Bifidobacterium longum, Bacteroides thetaiotaonicron, Treponema denticola, or a DGR from cyanobacteria. Non-limiting examples of such cyan bacteria include Trichodesmium erythraeum #1, Trichodesmium erythraeum #2, Nostoc PPC ssp. 7120 #1, Nostoc PPC ssp. 7120 #2, or Nostoc punctiforme.

In a further aspect, the invention provides a method of preparing a recombinant nucleic acid molecule as described herein by operably linking a first nucleic acid molecule comprising said VR to a second nucleic acid molecule comprising said TR such that said TR acts as a template sequence that directs site-specific mutagenesis of said VR. In the case of a linkage in cis between the VR and the TR, the first and second nucleic acid molecules would be covalently ligated together in a operative fashion as described herein. In the case of a linkage in trans, the first and second nucleic acid molecules would be placed in the same cellular environment or an in vitro reaction mix for site-specific mutagenesis in an operative fashion.

In yet another aspect, the invention provides a method of identifying additional RT coding sequences, IMH and IMH-like sequences, and corresponding TR and VR sequences. The method is based upon use of identified binding motifs of the RT activity of the invention to identify additional RT coding sequences in other organisms. The region near a putative additional RT coding sequence is then searched for nearby IMH type sequences which 1) are linked to putative TR sequences or 2) used to find VR linked IMH sequences.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein the sequence of said TR is an imperfect direct repeat of the sequence in said VR due to the substitution of one or more adenine nucleotides in said TR, or substitution of one or more non-adenine nucleotides in VR by adenines in TR, or substitution of VR adenine nucleotides by non-adenine nucleotides in TR.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR is all or part of a sequence encoding a binding partner of a target molecule.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR is all or part of a sequence encoding a binding partner of a target molecule, further comprising all of the sequence encoding said binding partner, wherein said VR is optionally the 3′ portion of said sequence encoding said binding partner.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR is all or part of a sequence encoding a binding partner of a target molecule, wherein said binding partner binds a cell surface molecule, a hormone, a growth or differentiation factor, a receptor, a ligand of a receptor, a bacterial cell wall molecule, a viral particle, an immunity or immune tolerance factor, or an MHC molecule.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR is all or part of a sequence encoding a binding partner of a target molecule, wherein said binding partner is a bacteriocin.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said TR and RT coding sequence are transcribed under the control of a heterologous promoter.

In another aspect the invention provides a cell containing a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other.

In another aspect the invention provides a method of preparing a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, said method comprising operably linking a first nucleic acid molecule comprising said VR to a second nucleic acid molecule comprising said TR such that said TR is a template sequence that directs site specific mutagenesis of said VR.

In another aspect the invention provides a method of preparing a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said TR and RT coding sequence are transcribed under the control of a heterologous promoter, said method comprising operably linking a heterologous promoter sequence to a nucleic acid molecule comprising said TR and RT coding sequence.

In another aspect the invention provides a method of site-specific mutagenesis of a nucleic acid sequence of interest, said method comprising: obtaining a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR comprises said nucleic acid sequence of interest and said TR is an imperfect or perfect repeat of said sequence of interest, wherein said TR is a template sequence operably linked to said sequence of interest to direct site-specific mutagenesis of the sequence, and wherein said TR is an imperfect repeat due to the substitution of one or more adenine nucleotide for a non-adenine nucleotide in said sequence of interest or visa versa; and allowing said nucleic acid molecule to be expressed in a cell such that one or more nucleotide positions of said sequence of interest is substituted by a different nucleotide.

In another aspect the invention provides a method of site-specific mutagenesis of a nucleic acid sequence of interest, said method comprising: obtaining a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR comprises said nucleic acid sequence of interest and said TR is an imperfect or perfect repeat of said sequence of interest, wherein said TR is a template sequence operably linked to said sequence of interest to direct site-specific mutagenesis of the sequence, and wherein said TR is an imperfect repeat due to the substitution of one or more adenine nucleotide for a non-adenine nucleotide in said sequence of interest or visa versa; and allowing said nucleic acid molecule to be expressed in a cell such that one or more nucleotide positions of said sequence of interest is substituted by a different nucleotide, wherein more than one nucleotide position of said sequence of interest is substituted.

In another aspect the invention provides a method of site-specific mutagenesis of a nucleic acid sequence of interest, said method comprising: obtaining a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR comprises said nucleic acid sequence of interest and said TR is an imperfect or perfect repeat of said sequence of interest, wherein said TR is a template sequence operably linked to said sequence of interest to direct site-specific mutagenesis of the sequence, and wherein said TR is an imperfect repeat due to the substitution of one or more adenine nucleotide for a non-adenine nucleotide in said sequence of interest or visa versa; and allowing said nucleic acid molecule to be expressed in a cell such that one or more nucleotide positions of said sequence of interest is substituted by a different nucleotide, wherein said sequence of interest encodes all or part of a binding partner of a target molecule.

In another aspect the invention provides a method of site-specific mutagenesis of a nucleic acid sequence of interest, said method comprising: obtaining a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR comprises said nucleic acid sequence of interest and said TR is an imperfect or perfect repeat of said sequence of interest, wherein said TR is a template sequence operably linked to said sequence of interest to direct site-specific mutagenesis of the sequence, and wherein said TR is an imperfect repeat due to the substitution of one or more adenine nucleotide for a non-adenine nucleotide in said sequence of interest or visa versa; and allowing said nucleic acid molecule to be expressed in a cell such that one or more nucleotide positions of said sequence of interest is substituted by a different nucleotide, wherein said sequence of interest encodes all or part of a binding partner of a target molecule, wherein the binding properties of said binding partner are altered.

In another aspect the invention provides an isolated nucleic acid molecule comprising a donor template region (TR) and an operably linked reverse transcriptase (RT) coding sequence, wherein the TR and RT coding sequence are heterologous to each other.

In another aspect the invention provides an isolated nucleic acid molecule comprising a donor template region (TR) and an operably linked reverse transcriptase (RT) coding sequence, wherein the TR and RT coding sequence are heterologous to each other, wherein the molecule is isolated from a bacteriophage, a prophage of a bacterium, a bacterium, or a spirochete.

In another aspect the invention provides a plurality or library of a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other.

In another aspect the invention provides a plurality or library of a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein the VR has undergone diversification directed by the TR.

In another aspect the invention provides a method of identifying initiation of mutagenic homing (IMH) sequences, said method comprising: identifying an RT coding sequence in a genome of an organism; searching the coding strand within about Skb of the RT ORF and identify an IMH-like sequence containing an 18-48 nucleotide stretch of adenine-depleted DNA; and a) using the putative IMH-like sequence to search genome-wide for a closely-related putative IMH and compare the DNA sequences located 5′ to the IMH-like and putative IMH sequences to find TR and VR regions, respectively; or b) using the sequence of the DNA located 100-350 base-pairs long 5′ to the IMH-like sequence to identify a putative TR, and use all or parts of this TR and IMH-like sequence to search genome-wide for a matching putative VR and IMH sequence.

In another aspect the invention provides a method of identifying initiation of mutagenic homing (IMH) sequences, said method comprising: identifying an RT coding sequence in a genome of an organism; searching the coding strand within about 5 kb of the RT ORF and identify an IMH-like sequence containing an 18-48 nucleotide stretch of adenine-depleted DNA; and a) using the putative IMH-like sequence to search genome-wide for a closely-related putative IMH and compare the DNA sequences located 5′ to the IMH-like and putative IMH sequences to find TR and VR regions, respectively; or b) using the sequence of the DNA located 100-350 base-pairs long 5′ to the IMH-like sequence to identify a putative TR, and use all or parts of this TR and IMH-like sequence to search genome-wide for a matching putative VR and IMH sequence, wherein said RT coding sequence is identified by searching for one or both amino acid sequences IGXXXSQ or LGXXXSQ; or wherein the IMH-like, or IMH, sequence contain a conserved sequence selected from TCGG, TTTTCG, or TTGT; or wherein the identified TR and VR sequences can be between about 100-350 base-pairs long and should be more than about 80% homologous, with the majority of differences being at the locations of the adenines bases in the TR.

In another aspect the invention provides a method of site-specific mutagenesis of a nucleic acid sequence of interest, said method comprising: obtaining a nucleic acid molecule comprising a donor template region (TR) and a variable region (VR), wherein said TR or VR or operably linked reverse transcriptase (RT) coding region is isolated from Vibrio harveyi ML phage, Bifidobacterium longum, Bacteroides thetaiotaonicron, Treponema denticola, or a cyanobacterial diversity generating retroelements (DGRs), and allowing said nucleic acid molecule to be expressed in a cell such that one or more nucleotide positions of said VR is substituted by a different nucleotide.

In another aspect the invention provides a method of site-specific mutagenesis of a nucleic acid sequence of interest, said method comprising: obtaining a nucleic acid molecule comprising a donor template region (TR) and a variable region (VR), wherein said TR or VR or operably linked reverse transcriptase (RT) coding region is isolated from Vibrio harveyi ML phage, Bifidobacterium longum, Bacteroides thetaiotaonicron, Treponema denticola, or a cyanobacterial diversity generating retroelements (DGRs), and allowing said nucleic acid molecule to be expressed in a cell such that one or more nucleotide positions of said VR is substituted by a different nucleotide, wherein said DGR is isolated from Trichodesmium erythraeum #1, Trichodesmium erythraeum #2, Nostoc PPC ssp. 7120 #1, Nostoc PPC ssp. 7120 #2, or Nostoc punctiforme.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein the 3′ end of the VR comprises about a 14 base pair element consisting of G and C residues, not not A residues.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein the 3′ end of the VR comprises about a 14 base pair element consisting of G and C residues, not not A residues, wherein about 4 to about 12 base pairs of the VR 5′ upstream, of and within about 350 base pairs of the base pair element have sequence homology with about 4 to about 12 base pairs of the TR.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein the VR comprises an initiation of mutagenic homing (IMH) sequence at its 3′ end.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein the TR consists essentially of about 10 to about 19 base pairs at its 5′ end and about 38 base pairs at its 3′ end.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein the TR is further extended upstream of the TR comprising the 3′ end of an atd region and the nucleotides between the TR and atd region; and the TR is further extended downstream of the TR comprising the 5′ end of a brt region and the nucleotides between the TR and brt region.

In another aspect the invention provides a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein the TR comprises an initiation of mutagenesis homing-like (IMH*) sequence at its 3′ end.

In another aspect the invention provides a method of site-specific mutagenesis of a nucleic acid sequence of interest, said method comprising: obtaining a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR comprises said nucleic acid sequence of interest and said TR is an imperfect or perfect repeat of said sequence of interest, wherein said TR is a template sequence operably linked to said sequence of interest to direct site-specific mutagenesis of the sequence, and wherein said TR is an imperfect repeat due to the substitution of one or more adenine nucleotide for a non-adenine nucleotide in said sequence of interest or visa versa; and allowing said nucleic acid molecule to be expressed in a cell such that one or more nucleotide positions of said sequence of interest is substituted by a different nucleotide, wherein the function of the TR comprises an RNA intermediate.

In another aspect the invention provides a method of site-specific mutagenesis of a nucleic acid sequence of interest, said method comprising: obtaining a single recombinant nucleic acid molecule or pair of nucleic acid molecules comprising: a variable region (VR) operably linked to a donor template region (TR), wherein said TR is operably linked to a reverse transcriptase (RT) coding sequence and is a template sequence that directs site-specific mutagenesis of said VR, and wherein the TR and RT coding sequence are heterologous to each other, wherein said VR comprises said nucleic acid sequence of interest and said TR is an imperfect or perfect repeat of said sequence of interest, wherein said TR is a template sequence operably linked to said sequence of interest to direct site-specific mutagenesis of the sequence, and wherein said TR is an imperfect repeat due to the substitution of one or more adenine nucleotide for a non-adenine nucleotide in said sequence of interest or visa versa; and allowing said nucleic acid molecule to be expressed in a cell such that one or more nucleotide positions of said sequence of interest is substituted by a different nucleotide, which is RecA-independent.

The details of one or more embodiments of the invention are set forth in the accompanying drawings and the description below. Other features, objects, and advantages of the invention will be apparent from the drawings, detailed description and from the claims.

### BRIEF DESCRIPTION OF THE DRAWINGS

FIGS. 1a to 1d show tropism switching by Bordetella bacteriophage. In FIG. 1a the specificities and tropism switching frequencies are depicted above the B. bronchiseptica BvgAS-mediated phase transition. BPP, BMP and BIP are tropic for Bvg+ phase, Bvg⁻ phase or either phase, respectively. FIG. 1b shows the components of the variability-generating cassette. The 3′ portion of mtd is expanded and the 134 bp VR sequence is underlined. Variable bases (red) correspond to adenine residues in TR. FIG. 1c shows that in wild type (wt) BPP-1, information is transferred unidirectionally from TR to VR and is accompanied by adenine-dependent mutagenesis. BPP-3′TR fails to switch tropism, whereas BPP-3′VR switches tropism at wild type frequencies and generates variability in TR as well as VR. In FIG. 1d, TR adenines are shown at the top followed by the corresponding nucleotides in the parental VR. TR1-9 are TR sequences derived from in vitro variability assays performed on phage BPP-3′VR. Red nucleotides show positions that varied. Sites of variability align with adenine residues in the parental TR.

FIGS. 2a and 2b show diversity-generating retroelements (DGRs) in bacterial and bacteriophage genomes. FIG. 2a shows a phylogenetic tree of DGRs in relation to other classes of retroelements. GenBank accession numbers are shown. DGR, diversity generating retroelements (red lines); G2, group II introns; Rpls, mitochondrial retroplasmids; Rtn, retrons; NLTR, non-LTR elements; LTR, LTR retroelements; Telo, telomerases; PLE, Penelope-like elements. RT domains were analyzed using the neighbor-joining algorithm of PHYLIP 3.6b, with 1000 bootstrap samplings, which are expressed as a percent. DGRs form a well-defined clade with 92% bootstrap support (red lines; Brt circled in pink). Group II introns are predicted to be their closest relatives, but with very weak support (55%). FIG. 2b shows nine putative DGRs in comparison to the Bordetella phage DGR. All DGRs include an ORF (191-888 aa) that contains a 103-190 bp VR (grey arrow) located at the C-terminus, a spacer region of 136-1,220 bp which in some cases contains a small open reading frame of similar size to atd, and a TR (black arrow) of equal length to VR in close proximity (22-339 bp) to RT (283-415 aa). For the Trichodesmium and Nostoc elements containing two VRs, VR1 and VR2 appear to have resulted from different mutagenic homing events originating from the same TR. E-values for RTs, in comparison to Brt, range from 1E-1 to 4E-37.

FIGS. 3a-3c show the results of multiple substitution experiments. In FIG. 3a, TR of phage MS1 contains synonymous substitutions marked with black lines (see Example I herein); TR adenines are marked with red lines with adjacent sites represented by a single line. Data boxed in purple or blue schematically represent the VR sequences of nine independent tropism variants. Purple box, BPP-MS1-->BMP or BIP; blue box, BMP-MS1-->BPP. A black line indicates that a substitution was acquired from TR; a red line indicates that a position varied with respect to the parental VR. The frequencies of transfer of synonymous substitutions (transmission histograms) are shown at the bottom. Purple bars, BPP-MS1-->BMP/BIP; blue bars, BMP-MS1-->BPP. FIG. 3b shows the results of in vitro variability assays (see Example 1 below) following selection for transfer of synonymous substitutions from TR to VR that confer resistance to MboII (position 100, boxed in purple) or AflIII (position 37, boxed in blue). Transmission histograms corresponding to the MboII selection (purple bars) or AflIII selection (blue bars) are shown at the bottom, along with positions of restriction enzyme cleavage (arrows). FIG. 3c shows that the TR of phage MS2 contains a 1 bp deletion at position 106 which, if transferred to VR, results in a frameshift mutation in mtd and non-infectious phage (see Methods). The data boxed in purple depict VR sequences of BPP-MS2-->BMP/BIP tropism variants. TR of phage MS3 contains a 1 bp deletion at position 9 which, if transferred to VR, results in non-infectious phage. The data boxed in blue show BMP-MS3-->BPP tropism variants. Transmission histograms corresponding to BPP-MS2-->BMP/BIP (purple bars) or BMP-MS3-->BPP (blue bars) reactions. Asterisks indicate the lack of transfer of frameshift mutations that are subject to negative selection.

FIGS. 4a and 4b show mosaic VR sequences result from mutagenic homing. In FIG. 4a, the average length of TR transferred under different selection conditions is shown with a histogram, and the distribution of transferred sequence lengths is depicted with bubbles (size represents the relative number of clones of a given length). Complex selections, such as those requiring a tropism switch (BPP-->BMP; BMP-->BPP), select for relatively rare isolates with longer stretches of transferred sequence. Simpler selections for transfer of single-nucleotide substitutions that result in restriction enzyme resistance (AflIIIs-->AflIIIr; MboIIs-->MboIIr) select for more abundant clones containing shorter stretches of transferred sequence, regardless of the point of selection. FIG. 4b shows the generation of VR sequences containing random portions of TR of variable length. In the model proposed with the instant invention, reverse transcription is followed by mutagenic homing, in which a TR-derived reverse transcript integrates in a homology-dependent manner at VR forming a heteroduplex. This event could initiate at the IMH site and occur by a mechanism analogous to target-primed reverse transcription (TPRT), as proposed for group II introns (Morrish, T. A. et al. DNA repair mediated by endonuclease-independent LINE-1 retrotransposition. Nat Genet. 31, 159-165 (2002) and Wank, H., SanFilippo, J., Singh, R. N., Matsuura, M., Lambowitz, A. M. A reverse transcriptase/maturase promotes splicing by binding at its own coding segment in a group II Intron RNA. Mol Cell 4, 239-250 (1999)). The resulting heteroduplex would contain a high density of mismatched base pairs (red asterisks) due to adenine-specific mutagenesis. The heteroduplex is then partially converted to the parental VR sequence via mismatch repair, and/or recombination. DNA replication would produce mosaic VRs with patches of TR-derived variable sequence.

FIG. 5 shows the use of in-frame deletions to define the boundaries of the BPP-1 diversity-generating cassette. Internal in-frame deletions were introduced into phage genes flanking the brt-mtd region. A map of the BPP-1 genomic segment containing the tropism switching region is shown, along with phenotypes resulting from in-frame deletions. Viability is defined as the production of infectious phage particles following induction of lysogens with mitomycin C. Variability is defined as the production of phage DNA containing adenine mutagenized VR sequences following induction of lysogens using detected with in vitro variability assays. Phage genes bbp1, bbp2, bbp3, and bbp4 are all essential for BPP-1 viability, but unnecessary for VR variability. Phage genes brt, atd, and mtd are all necessary for VR variability in these constructions. Of these three variability cassette genes, only mtd is essential for BPP-1 viability. Phage genes bbp9 and bbp10 are not required for variability or viability. All variability determinants identified to date lie within a defined, continuous region of the phage genome, supporting the idea that the variability-generating loci function as a cassette.

FIG. 6, parts a-c, show the results of adenine-dependent mutagenesis of TR. Part a: the top sequence shows a TR with 23 naturally occurring adenines (bold) and an additional ectopic adenine residue introduced at a new site by site-specific mutagenesis followed by allelic exchange (position 55, red bold). VR1-VR5 show VR sequences from independently isolated tropism variants in which the ectopic adenine was observed to vary. The actual frequency of variability at the ectopic adenine is shown in part c. These data demonstrate that ectopic addition of an adenine residue in TR creates a new site of variability in VR. Part b: the top sequence shows a TR in which a naturally occurring adenine pair at position 23-24 has been substituted with GC (red bold). The remaining 21 naturally occurring adenines are in bold. Out of 20 independently isolated tropism variants, of which a representative 5 are shown (VR1-VR5), no variability was observed at positions 23-24. Since the frequency of alteration of the naturally occurring adenine pair at position 23-24 during tropism switching is ˜95%, the elimination of adenine residues in TR eliminates variability at the corresponding position in VR. Part c: frequencies of mutagenesis at transmitted adenines were calculated using in vitro variability assays. The frequency of mutagenesis at pairs of transmitted adenines resulting in a substitution at either position (AA->NA/NN; AA->AN/NN) or both positions (AA->NN) is shown (n=20). Mutagenesis frequencies at the single endogenous adenine at position 35 (endogenous A->N, n=50) or the ectopic adenine at position 55 (ectopic A->N, n=50) are also shown. As observed and provided within the scope of the invention, an adenine that is part of a pair is much more likely to vary than a single adenine, and the frequency of variability at the ectopic adenine at position 55 (see Part a above) is nearly identical to that for the endogenous adenine at position 35.

FIG. 7, parts a and b, show the results of internal deletion experiments. Part a: stretches of sequence were deleted from TR and VR of BPP-1 as indicated on the diagram (to scale) and the resulting strains were tested for variation in VR using in vitro variability assays. Variation in VR is indicated by “+” in the column to the right, while lack of variation is indicated by a “−”. Except for very large deletions (D118), the system was able to accommodate deletions of different size and location (Δ18, Δ39, Δ61). Most significantly, a large deletion of the 5′ portion of VR (Δ61) still displayed variation, indicating that there is no 5′ cis-acting site analogous to IMH and that homing in this system is in part based on homology. Part b: sequences of variant VRs (VR1-4) derived from A61 phage are aligned against TR and VR (above). The sequence between the deletion and the G/C stretch is shown. The MboII site of selection is also shown (underlined), together with mutagenesis (red) at residues corresponding to adenines in TR (bold).

FIG. 8, parts a and b, show the tropism switching frequencies of phage carrying multiple substitutions in TR. Strain abbreviations are the same as in FIG. 3. MS1 carries 5 synonymous substitutions while MS2 and MS3 carry a 1 bp deletion in addition to synonymous substitutions (see maps in FIG. 3). Part a: multiple substitution constructs in the BMP-1 background (MS1, MS2, MS3) or wild type BMP-1 were selected for switching to the BPP tropism. Phage induced from lysogens were propagated on Bvg⁻ bacteria and the fraction of phage able to form plaques on Bvg+ was measured. Part b: multiple substitution constructs in the BPP-1 background or wild type BPP-1 were selected for switching to the BMP or BIP tropisms. Phage induced from lysogens were propagated on a Bvg+ host and the fraction of phage able to form plaques on a Bvg⁻ host was measured. In parts a. and b., the frequencies of tropism switching for MS2 and MS3 phages are lower than wild-type, indicating that a fraction of phage was eliminated by negative selection. In both cases, however, these mutant phages were able to switch tropism while avoiding the transmission of frameshift mutations (FIG. 3c).

FIG. 9 shows the nucleotide sequence alignments of VRs and TRs from different DGRs. TR sequence is shown on top with VR sequence(s) on the bottom. Stop codons are shown in lower case. Adenines in TR are shown in bold, while the corresponding bases in VR are boldfaced only if different from TR. Note that the differences are largely limited to TR adenines, as opposed to non-adenine substitutions, indicating that the basic mechanism of mutagenesis is conserved across DGRs. Mismatches at the 3′ end, similar to IMH in Bordetella phage, are shown in color (green, VR; blue, TR). In addition, a well-conserved TCTT motif at the 3′ end, whose functional significance is unclear, is underlined. These similarities attest to likely conservation of mechanistic features, despite the lack of sequence identity between the different elements.

FIG. 10 shows schematics representing constructs of the invention. In the first construct, an atd region is present between the 3′ end of the indicated terminator and the start of the TR region. In the second construct, the atd region is present between the promoter and the indicated TR region. In the third construct, no atd or TR region is present in the construct.

FIG. 11 shows mutagenesis of VR on an induced prophage.

FIG. 12 shows an illustration of the design to mutagenize a heterologous sequence with a novel TR and IMH.

FIG. 13 shows an illustration of constructs used to mutagenize a phosphotransferase encoding sequence.

FIG. 14 shows the VR amino acid sequence used in the mutagenesis of a non-Bordetella APH(3′)-IIa encoding sequence. The large “L” delineates the location of the insertion of an amber codon at position 243 for the elimination of kanamycin binding and inactivation of kanamycin resistance.

FIG. 15 shows an alignment of sequences from various DGRs (including Cyanobacterial DGRs, and those from Nostoc punctiforme, Nostoc spp. 7120 #1 & #2, Trichodesmium Erythraeum #1 & #2 and others) of the invention.

FIG. 16 shows DGR homing occurs through an RNA intermediate.

FIG. 16A shows the Bordetella phage DGR cassette undergoes mutagenic homing and facilitates tropism switching: mtd, atd, brt and the two repeats (VR and TR) are indicated. VR and TR are expanded to show the (G/C)₁₄, IMH and IMH* elements. DGR homing leads to VR diversification, resulting in progeny phages with altered Mtd trimers at the distal ends of tail fibers.

FIG. 16B shows the PCR-based DGR homing assays. Plasmids pMX-TG1a, b, c are derived from pMX1b, which carries the BPP-1 atd-TR-brt region placed downstream of the BvgAS-regulated fhaB promoter. pMX-TG1a, b, c contain 36 bp inserts (TG1), corresponding to ligated exons of the T4 td group intron flanked by SalI sites, at TR positions 19 (TG1a), 47 (TG1b) or 84 (TG1c), respectively. Grey and pink arrows represent TR and VR, respectively. Small horizontal arrows indicate primers used for homing assays: P1, sense-strand primer located in mtd; P2, antisense-strand primer downstream of VR; P3 and P4 are sense- and antisense-strand primers, respectively, that anneal to ligated td exons. Cam^{R}, chloramphenicol resistance gene.

FIG. 16C shows the pMX-TG1a, b, c are functional donors for DGR homing. Assays were performed following BPP-1d single-cycle lytic infection of B. bronchiseptica RB50 cells transformed with the indicated donor plasmids. pMX1b (negative control) lacks the TG1 insert. Larger PCR products (*) in lanes 2-4 are Brt-independent PCR artifacts (FIG. 18, lanes 1 & 2 and data not shown). They contain sequences from mtd to TR, have not undergone adenine mutagenesis, and were likely generated by PCR template switching between BPP-1 d DNA and contaminating donor plasmids in phage DNA preparations. P1+P2 product levels demonstrate that approximately equal amounts of input DNA were used for PCR homing assays.

FIG. 16D shows testing the DGR retrohoming hypothesis. The T4 td group I intron (td) and flanking exons (E1, E2) were inserted at position 84 in TR. Following transcription and intron splicing, ligated exons will be retained in a subset of TR-containing transcripts. If homing occurs via an RNA intermediate (retrohoming), some VRs will acquire ligated exons. Primers used for PCR homing assays are indicated by small horizontal arrows: P1 and P2 are described in (B); E1s, sense-strand primer annealing to E1; E2a, antisense-strand primer annealing to E2.

FIG. 16E shows the precisely ligated td exons are transferred to VR during homing. Products from PCR homing assays with indicated primer pairs and donor plasmids are shown. TG1c, pMX-TG1c positive control; td, pMX-td donor plasmid; td/SMAA, RT-deficient pMX-td derivative; P6M3 and ΔP7.1-2a, splicing-defective pMX-td derivatives; td−, pMX1b with the td intron and flanking exons inserted in inverted orientation at TR position 84. *, Brt-independent PCR artifacts.

FIG. 17 shows internal TR sequences are dispensable for homing.

FIG. 17A shows the TR deletion constructs and homing activities. All donor plasmids are derivatives of pMX1b, containing AvrII (Av) and Apal (Ap) sites in atd and brt, respectively, as well as a sequence tag (TG2). AvrII and Apal sites were introduced via silent mutations and do not affect DGR homing (FIG. 28). FL (full-length) is a positive control with TG2 inserted at position 84. ΔTR1-84, ATR 1-84, ΔTR23-84, ΔTR33-84, ΔTR33-96 and ΔTR33-113 contain TR deletions with TG2 at deletion junctions. Results from PCR homing assays are summarized: +, homing efficiency similar to the FL positive control; ++, homing activity greater than FL; −, no homing activity detected.

FIG. 17B shows the primers used for homing assays. P5 and P6 are sense- and antisense-strand primers annealing to TG2, respectively; primers P1 and P2 are described in FIG. 16.

FIG. 17C shows the PCR homing assays with TR deletion constructs. Homing assays were performed following BPP-1d single-cycle lytic infection of RB50 cells transformed with the indicated plasmids. *, Brt-independent PCR artifacts.

FIG. 18 shows homing does not require RecA.

DGR homing assays were performed following BPP-1d single-cycle lytic infection of RB50 or RB50ArecA cells transformed with the indicated plasmids. TG1c/SMAA, RT-deficient mutant of pMX-TG1c (FIG. 16B); other donor plasmids have been described (FIG. 16E). Primers used in homing assays are shown below the gel. *, Brt-independent PCR artifacts.

FIG. 19 shows marker conconversion analysis of DGR retrohoming.

FIG. 19A shows the strategy to identify cDNA integration sites at the 3′ and 5′ ends of VR via marker coconversion. Markers introduced into TR (red Ts) are transferred to VR only if they are located between 3′ cDNA priming and 5′ cDNA integration sites.

FIG. 19B shows the schematic of coconversion experiments. Single C to T markers in donor TRs are indicated. All TRs are tagged with TG1 at position 84.

FIG. 19C shows the homing assays were performed following BPP-1d single-cycle lytic infection of RB50 cells transformed with marked donor plasmids. Results from PCR homing assays are shown in FIG. 32 and marker coconversion data are summarized here. Nucleotides at relevant positions in the wild type parental VR are shown in the center. At bottom, the number of progeny VRs with a transferred marker, or no transferred marker at specific sites are shown. Deduced cDNA integration regions at the 3′ and 5′ ends of VR are indicated by brackets.

FIG. 20 shows cDNA integration at the 3′ End of VR.

FIG. 20 (A) shows the experimental design. Donor plasmid pMX-td contains the td group I intron at position 84 in TR, and prophage BPP-1dΔVR1-99 lacks the first 99 bp of VR. cDNA integration at the 3′ or 5′ end of VR was assessed in PCR assays using the same primer pairs described in FIG. 16D.

FIG. 20 (B) shows the VR lacking the first 99 bp supports cDNA integration at the 3′ end, but not the 5′ end. BPP-1dΔVR1-99 (IMH) and BPP-1dΔVR1-991MH* (IMH*) lysogens transformed with the indicated donor plasmids (described in FIG. 16E) were induced with mitomycin C for 2 hrs. Total nucleic acids isolated from induced cultures were assayed for cDNA integration by PCR using primers shown in (A). Pink arrow, cDNA intermediate. *, Brt-independent PCR artifacts.

FIG. 21 shows cDNA integration at the 5′ End of VR

FIG. 21A shows the strategy to identify requirements for cDNA integration at the 5′ end of VR. Donor plasmid pMX-M50 is a derivative of pMX-ΔTR23-84 (FIG. 17A) containing an insert in TR consisting of a 50 bp mtd segment (M50, derived from sequences located upstream of VR). The M50 insert in TR provides homology to the mtd locus in prophage BPP-1dΔVR1-99. Primers are indicated as small horizontal arrows: P7 is a sense-strand primer annealing to mtd, P2, P5 and P6 are described in FIG. 16 and FIG. 17. Int., integration.

FIG. 21B shows the M50 insert in TR restores homing into sequences upstream of VR in BPP-1dΔVR1-99. PCR assays were performed on DNA isolated from intact phage particles produced following induction by mitomycin C. RB50 lysogens carried BPP-1d prophage genomes with wild type VR sequences (VRwt) or the VR1-99 deletion (ΔVR1-99). Donor plasmids were as follows: ΔTR23-84, pMX-ΔTR23-84; M50, pMX-M50; M50/SMAA, an RT-deficient mutant of pMX-M50. Product bands 1 and 2 in lane 2 and band 3 in lane 5 are labeled. *, Brt-independent PCR artifacts.

FIG. 21C shows the major products derived from bands 1, 2 and 3 in (B) are shown and described in the text. Yellow bars, VR1-99 or portions thereof.

FIG. 22 shows a model for DGR mutagenic homing. DGR homing occurs via a “copy and replace” pathway that substitutes parental VR sequences with diversified cDNA copies of TR transcripts. cDNA integration at the 3′ end of VR is proposed to occur via a TPRT mechanism, while integration at the 5′ end of VR requires short stretches of TR/VR sequence homology and occurs through template-switching and/or strand displacement. Adenine mutagenesis most likely occurs during minus-strand cDNA synthesis by the DGR-encoded RT. The resulting mismatches in VR DNA strands could be resolved via DNA replication. Pink arrows and bars, cDNAs and cDNA-derived sequences. “N”, any of the four deoxyribonucleotides. Dashed lines, TR flanking sequences.

FIG. 23 shows that the alignment of plasmid pMX-TG1a homing products with TG1a TR sequences demonstrates adenine mutagenesis.

FIG. 23A shows the PCR detection strategy for pMX-TG1a homing products and regions of the products aligned in B and C. Primer annealing sites are indicated as small horizon arrows.

FIG. 23B shows that the alignment of the transferred TG1 tag and its upstream VR sequence to the corresponding TR region shows adenine mutagenesis.

FIG. 23C shows that the alignment of the transferred TG1 tag and its downstream VR sequences to the corresponding TR region shows adenine mutagenesis.

FIG. 24 shows that the alignment of plasmid pMX-TG1b homing products with TG1b TR sequences demonstrates adenine mutagenesis.

FIG. 24A shows the PCR detection strategy for pMX-TG1b homing products and areas of the products aligned in B and C. Primer annealing sites are indicated as small horizontal arrows.

FIG. 24B shows that the alignment of the transferred TG1 tag and its upstream VR sequence to the corresponding TR region shows adenine mutagenesis.

FIG. 24C shows that the alignment of the transferred TG1 tag and its downstream VR sequence to the corresponding TR region shows adenine mutagenesis.

FIG. 25 shows that the alignment of plasmid pMX-TG1c homing products with TG1c TR sequences demonstrates adenine mutagenesis.

FIG. 25A shows the PCR detection strategy for pMS-TG1c homing products and areas of the products aligned in B and C. Primer annealing sites are indicated as small horizontal arrows.

FIG. 25B shows that the alignment of the transferred TG1 tag and its upstream VR sequence to the corresponding TR region shows adenine mutagenesis.

FIG. 25C shows that the alignment of the transferred TG1 tag and its downstream VR sequence to the corresponding TR region shows adenine mutagenesis.

FIG. 26 shows that the phage T4 td intron self-splices in B. bronchiseptica.

FIG. 26A shows the analysis of td intron RNA splicing by RT-PCR. Assays were performed with total RNAs isolated from B. bronchiseptica RB50 cells transformed with the indicated plasmids. Reverse transcription reactions with RNA samples were carried out with primer P8, and cDNA products were then amplified with primers P9 and P10 as diagramed below the gel. RT-PCR products of precursor and spliced RNAs are indicated to the left, and are 632 and 238 bp, respectively. Detection of these products required the presence of superscript III RT in the reverse transcription reaction. Sequence analysis of the spliced product from pMX-td (lane 2) did not show any sign of adenine mutagenesis (data not shown), suggesting that adenine-directed sequence diversification does not occur at the RNA level. TG1c, positive control pMX-TG1c; td. pMX-td; td/SMAA, Brt-deficient mutant of pMX-td; P6M3 and ΔP7.1-2a, splicing-defective mutants; td−, donor with the td intron inverted.

FIG. 26B shows the quantitative analysis of td intron splicing through primer-extension-termination assays. 5′-³²P-labeled primer tdE2 was used for primer extension assays in the presence of dATP, dCTP, dGTP and ddTTP (dideoxythymidine triphosphate), which terminates cDNA extension once incorporated. As the first adenine residues are located at different distances from the primer annealing sites in precursor and spliced RNAs, products of different sizes are produced and resolved by denaturing polyacrylamide gel, allowing accurate measurement of relative amounts of precursor and spliced RNAs. The primer and its extension products are indicated to the left. Donor plasmids are described in A. The spliced products of pMX-td and the Brt-deficient pMX-td/SMAA are 18% of the transcript levels fo the positive control pMX-TG1c, which contains ligated td exons in TR.

FIG. 27 shows that the alignment of plasmid pMX-td homing products with TG1c TR sequences shows precise exon ligation and adenine mutagenesis.

FIG. 27A shows the PCR detection strategy for pMX-td homing products and areas of the products aligned in B and C. Primer annealing sites are indicated as small horizontal arrows.

FIG. 27B shows that the alignment of the transferred td exons and their upstream VR sequence to the corresponding TG1c TR region shows accurate exon joining and adenine mutagenesis. Primer E2a annealing site and exon junction (EJ) are also indicated.

FIG. 27C shows that the alignment of the transferred td exons and their downstream VR sequences to the corresponding TG1c TR region shows accurate exon joining and adenine mutagenesis. Primer E1s annealing site, EJ and the (G/C)₁₄ element are indicated.

FIG. 28 shows that the silent mutations used to introduce Avril and Apal sites in atd and brt do not affect DGR function.

FIG. 28A shows the donor plasmid pMS-TG1c/AA and its homing product. pMX-TG1c/AA is essentially the same as pMX-TG1c except for the Avril (Av) and Apal (Ap) sites introduced by silent mutations.

FIG. 28B shows that the introduction of the Avril and Apal sites in atd and brt has no detectable effect on DGR homing. Homing assays were performed as in FIG. 16C. TG1c, positive control pMX-TG1c/SMAA, Brt-deficient derivatives of pMX-TG1c; TG1c/AA, pMX-TG1c/AA, *, Brt-independent PCR artifact.

FIG. 29 shows that the TR sequences between TG1a, TG1b and TG1c tag insertion sites are not required for DGR homing.

FIG. 29A shows the TR internal deletion constructs that delete regions between the tag insertion sites of TG1a and TG1c (ΔTR20-84), TG1b and TG1c (ΔTR48-84), and TG1a and TG1b (ΔTR20-47). The TG1 tag was inserted at the deletion junctions in all the constructs to facilitate homing assays. Homing activities analyzed in C are summarized to the right. +, homing activity of the positive control pMX-TG1c; ++, activities significantly above the positive control.

FIG. 29B shows the homing products and assay primers.

FIG. 29C shows the homing assays of TR internal deletion constructs in A. Assays were performed as in FIG. 16C. TG1c, positive control pMS-TG1c; TG1c/SMAA. Brt-deficient negative control. *, Brt-independent PCR artifact.

FIG. 30 shows silent mutation scanning to detect regions of the TR-containing RNA transcript important for DGR homing.

FIG. 30A shows the locations of silent mutations in the donor constructs and their homing products. A1-3 are three different donors with silent mutations at the 3′ end of atd, downstream of the Avril (Av) site. T7-9 are three donors with silent mutations at the 5′ end of brt, upstream of the Apal (Ap) site. The brt ORF can potentially be extended at the 5′ end to include the entire TR. T1-6 are donors with silent mutations in this extended ORF. The mutations in T1-3 are ocated in TR, while those in T4-6 are located in the spacer between TR and brt. Primers were described in FIG. 16B.

FIG. 30B shows the effects of silent mutations A1-3 and T1-9 on DGR homing. Homing assays were performed as in FIG. 16C. TG1c/AA, positive control pMX-TG1c/AA. *, Brt-independent PCR artifact.

FIG. 31 shows that the DGR-mediated phage tropism switching occurs independently of the host RecA-dependent homologous recombination function. Progeny phages were generated from phage BPP-1d through single-cycle lytic infection of wild type RB50 and RB50ΔrecA cells harboring the homing-competent donor plasmid pMX1. Tropism switching frequencies were determined as the ratio of progeny phages that infect Bvg phase RB54 cells vs. those that infect Bvg+ phase RB53 Cm cells. Data represent the mean of two independent experiments±standard deviation. As negative controls, the Brt-deficient donor pMX1/SMAA failed to support BPP-1d phage tropism switching in either cell type with at least 2.8×10⁹ progeny phages analyzed (data not shown).

FIG. 32 shows the DGR homing assays to detect marker coconversion of donor VRs with different C to T markers in TR.

FIG. 32A shows the donor plasmid pMX-TG1c/AA that was used for introduction of different C to T markers and its homing product.

FIG. 32B shows the donor plasmids containing different C to T markers in TR support DGR homing. TG1c/AA, positive control pMX-TG1c/AA. Other donors contain C to T markers at the indicated TR positions. *, Brt-independent PCR artifact.

FIG. 33 shows that the sequence alignment of potential homing intermediates generated in BPP-1dΔVR1-99 lysogens expressing pMX-td show precise exon ligation and adenine mutagenesis.

FIG. 33A shows a potential DGR homing intermediate and its detection by PCR. Homing intermediates were amplified with primers E1s and P2, and subsequently cloned and sequenced. Area of interest is aligned in B.

FIG. 33B shows that the alignment of sequences of 6 independent cDNA clones with the corresponding TG1cTR sequence shows accurate exon joining and adenine mutagenesis. Primer E1s annealing site, exon junction (EJ) and the (G/C)₁₄ element are also indicated.

FIG. 34 shows the sequences of products a and b from band 1 in lane 2 of FIG. 21B.

FIG. 34A shows at top a diagram of product a, generated with primers P7 and P6. Area of interest aligned below is also indicated. 3 independent clones of product a are aligned with the corresponding TR sequence of pMX-M50 to show adenine mutagenesis and to determine 5′ cDNA integration sites. Primer P6 annealing site and M50 are also indicated. As the BgIII site in TR was also transferred to VR and diversified, cDNA integration most likely occurred within the first 22 bp of the parental VR. B′, diversified BgIII site.

FIG. 34B shows at top a diagram of product b. The 8-nt sequence implicated in mediating 5′ cDNA integration is shown. Area of interest aligned below is also indicated. 8 independent clones of product b are aligned. Judging from the boundary of phage- and plasmid-donated sequences, 5′ cDNA integration appears to have occurred between VR positions 60 and 67 (shown in red), and was mediated by the homologous 8 nt sequence in TR of pMX-M50.

FIG. 35 shows the sequences of products c and d of band 2 in lane 2 of FIG. 21B.

FIG. 35A shows at top a diagram of product c, generated with primers P7 and P6. Area of interest aligned below is indicated. 7 independent clones of product c are aligned with the corresponding TR sequence of pMX-M50 to show adenine mutagenesis and to determine 5′ cDNA integration sites. As the BGIII site in TR was not transferred to VR and adenine mutagenesis occurred within the M50 insert, cDNA integration most likely occurred within the M50 sequence of the phage mtd gene.

FIG. 35B shows at top a diagram of product d. The 9 nt sequence implicated in mediating 5′ cDNA integration upstream of M50 in the mtd gene is shown. Area of interest aligned below is also indicated. 5 independent clones of product d are aligned with the corresponding TR sequence of pMX-M50 to show adenine mutagenesis and to determine 5′ cDNA integration sites. As the BgIII site was transferred from TR to VR and in two cases, diversified, and no TR sequences upstream of the 9 nt homology were transferred, 5′ cDNA integration is concluded to have occurred within the 9 nt homologous sequence located 6 nt upstream of M50 in the phage mtd gene. B′, diversified BgIII site.

FIG. 36 shows the alignment of pMX-M50 homing products in BPP-1d lysogens (detected in lane 8 of FIG. 21B) with the corresponding TR sequence of the donor plasmid. Shown at top is a diagram of the homing product detected with PCR primers P5 and P2 in lane 8 of FIG. 21B. Area of interest aligned below is indicated. Shown below is alignment of 111 independent homing products with the corresponding TR region of pMX-M50. (G/C)₁₄/IMH elements and primer P5 annealing site are indicated.

FIG. 37 shows the sequences of products e and f of band 3 in lane 5 of FIG. 21B.

FIG. 37A shows at top a diagram of product e, which has a structure identical to product c. Area of interest aligned below is indicated. 4 independent clones of product e are aligned with the corresponding TR sequence of pMX-M50 to show adenine mutagenesis and to determine 5′ cDNA integration sites. The BgIII site in TR was not transferred to VR and adenine mutagenesis occurred within the M50 insert in two of the clones, indicating that at least two of the clones are true homing products and that cDNA integration occurred within the M50 sequence of the phage mtd gene.

FIG. 37B shows at top a diagram of product f that is structurally identical to product d. 11 independent clones of product f are aligned with the corresponding TR sequence of pMX-M50. As the BgIII site was transferred from TR to VR and in 6 cases, diversified, and no TR sequences upstream of the 9 nt homology were transferred, we conclude that 5′ cDNA integration occurred within the 9 nt homologous sequence. B′, diversified BgIII site.

FIG. 38 shows the alignment of pMX-M50 homing products in BPP-1dΔVR1-99 lysogen (detected in lane 11 of FIG. 21B) with the corresponding TR sequence of the donor plasmid. Shown at top is a diagram of the homing products detected with PCR primers P5 and P2 in lane 11 of FIG. 21B. Area of interest aligned below is indicated. Shown below is an alignment of 11 independent homing products with the corresponding TR sequence of pMX-M50. (G/C)₁₄/IMH elements and primer P5 annealing site are indicated.

### DETAILED DESCRIPTION OF THE INVENTION

This invention provides nucleic acid molecules and methods for their use in site specific mutagenesis of a sequence of interest which is in whole or in part the VR in a operative linkage between the VR and a homologous repeat (TR) that directs the diversification of the sequence of interest at positions occupied by adenines within the TR. The extent of diversity that can be generated by the invention is not equal to the number of adenine positions that are capable of directing substitutions in the VR. Instead, each adenine in TR can result at that position in 3 different nucleotide substitutions in the VR, many of which will result in a substituted amino acid at the corresponding position encoded by the VR. As a non-limiting example, the presence of 23 adenine nucleotides in the practice of the invention is theoretically capable of generating over 1012 distinct polypeptide sequences.

Thus the invention provides for the presence of up to 23 or more adenine nucleotides in a given TR of the invention to direct mutagenesis in the corresponding VR. The presence of adenine residues may be due to natural occurrence in the TR or the result of deliberate insertion or substitution into the TR as described herein. In the case of naturally occurring adenine nucleotides in the TR, mutagenesis may be allowed to occur or may be avoided by a substitution of the adenine nucleotide to a non-adenine nucleotide without changing the encoded amino acid (silent substitution). In the case of deliberate insertion or substitution, the invention provides for the introduction of 1, 2, 3, 4, 5, 6, 7, 8, 9, 10, 11, 12, 13, 14, 15, 16, 17, 18, 19, 20 or more adenine nucleotides into a TR.

As described herein, the invention provides recombinant and isolated nucleic acid molecules comprising a variable region (VR) which is operable linked to a template region (TR), wherein the VR and TR sequences are in the same molecule or separate molecules, and wherein said TR is a template sequence operably linked to said VR in order to direct site specific mutagenesis of said VR. Preferably, however, the molecule is not a derivative, containing only one or more deletion mutations, of the major tropism determinant (mtd) gene, the atd region, and/or the brt coding sequence, of Bvg+ tropic phage-1 (BPP-1) bacteriophage.

The VR and TR regions may be physically and operably linked in cis or operably linked in trans as described herein. The separation between the two regions when linked in cis can range from about 100 base pairs or less to about 1200 base pairs or more. When associated via a cis or trans configuration, expression of the TR and operably linked RT coding sequence may be under the control of an endogenous or heterologous promoters. When associated in trans, expression of the TR and operably linked RT coding sequences may be under the control of an endogenous or heterologous, regulatable promoter or promoters.

The nucleic acid molecules of the invention may also contain an RT encoding region in cis with the TR region. Non-limiting examples of RT coding sequences include those from Vibrio harveyi ML phage, Bifidobacterium longum, Bacteroides thetaiotaonicron, Treponema denticola, or a DGR from cyanobacteria, such as Trichodesmium erythrism, the genus Nostoc, or Nostoc punctiforme as provided herein. The relevant RT econding sequences from these sources are all publicly accessible and available to the skilled person. Additionally, some nucleic acid molecules may contain an atd region (or bbp7 region) immediately 5′ of the TR. Without being bound by theory, and offered to improve the understanding of the invention, the atd region is believed to participate in regulating transcription of the TR and so may be augmented by use of a heterologous promoter.

In embodiments of the invention comprising the use of a heterologous promoter, the promoter may be any that is suitable for expressing the TR and RT coding sequence under the conditions used. As a non-limiting example, when a prokaryotic cell is used with the VR and TR regions, the promoter may be any that is suitable for use in the prokaryotic cell. Non-limiting examples include the filamentous haemagglutinin promoter (fhaP), lac promoter, tac promoter, trc promoter, phoA promoter, lacUV5 promoter, and the araBAD promoter. When the conditions are those of a eukaryotic cell, non-limiting examples of promoters include the cytomegalovirus (CMV) promoter, human elongation factor-1E promoter, human ubiquitin C (UbC) promoter, SV40 early promoter; and for yeast, Gal 11 promoter and Gal 1 promoter. Of course, the VR may remain under the control of an endogenous promoter, if present, or be under the control of another heterologous promoter independently selected from those listed above or others depending on whether a prokaryotic or eukaryotic cell is used. If a cell-free system is used in the practice of the invention, then the promoter(s) will be selected based upon the source of the cellular transcription components, such as RNA polymerase, that are used.

The nucleic acid molecules of the invention may also contain an IMH sequence or a functional analog thereof. The function of the IMH has been described above, and the invention further provides for the identification, isolation, and use of additional functionally analogous sequences, whether naturally occurring or synthetic. In the case of naturally occurring functional analogs, they may be used with heterologous VR and TR sequences in the practice of the instant invention.

Non-limiting examples of IMH and IMH-like sequences for use in the practice of the invention include those shown in the following Table. An IMH or IMH-like sequence may contain the GC-rich region through the 3′ end.

TABLE 1

| | GC-rich region (50-91% GC); length (4-31 nt) | mismatches | |
|---|---|---|---|
| | TC or TTGG . . . start | (1-5) length | |
| VR 3′-end | nucleotide runs (3-7 nt) | (1-9 nt) | IMH 3′ end |
| BPP1 | | | |
| TR GCGAACA- | TCGG-GGCGCGCGGCGTCTGTG (81% GC) | CCCATCACC | TTCTTG |
| VR GCGTTCT- | TCGG-GGCGCGCGGCGTCTGTG (21 nt) | ACCACCTGA | TTCTTGAGtag |
| B. Longum | | | |
| TR TGGAACA- | TCGG-GGGCCGC (91% GC) | ATATCC | G |
| VR TGGCACC- | TCGG-GGGCCGC (11 nt) | CTTTCT | GCGCTCGGTCGCACGAAGGCGtag |
| Bacteriodes T. | | | |
| TR ACAACAA- | TCGG-GCGTACGGGTTTGGG (68% GC) | G | TGCGTTCTTCCCAAGAAT |
| VR ACTACTC- | TCGG-GCGTGCGGGTTTGGG (19 nt) | T | TGCGTTCTTCCCAAGAAtag |
| Vibrio Harveyi | | | |
| TR AATAGCA- | TCGG-TTTTCGCCCCGCT (65% GC) | CTTGA | TGT |
| VR AGTAGCA- | TCGG-TTTTCGCCCCGCT (17 nt) | TTCTT | TGTGtaa |
| T. denticola | | | |
| TR GACAACAA- | TCTT-GGCTTCCGCTTGGCTTG (57% GC) | TCGGCCC | |
| VR TGCAGCGA- | TCTT-GGCTTCCGCCTGGCTTG (21 nt) | CCGGCCT | taa |
| Trichodesmium | | | |
| Erythraeum #2 | | | |
| TR CGAGTCA- | TCTCGTCTTCCCCGGTGGTTTCTGGCTTTCATTCCTAGTATTCTT | | |
| | C | | |
| VR CGAGTCA- | TCTCCTCTTCCCCGGTGGTTTCTGGCTTTCATTCCtagTATTCTT | | |
| | C | | |
| Trichodesmium | | | |
| Erythraeum #1 | | | |
| TR CAACAATA- | TTGGTTTTCGT-CTTGT-GAGTTTCCCCCCCAG (52% GC) | C | ACTCTT |
| VR1 CATCAATT- | TTGGTTTTCGT-CTTGT-GAGTTTCCCCCCCAG (31 nt) | G | ACTCTTGAAtag |
| VR2 CGACTTTG- | TTGGTTTTCGT-CTTGT-GAGTTTCCCCCCCAG | G | ACTCCtga |
| Nostoc spp. 7120 #1 | | | |
| TR AACAATA- | TTGGTTTTCGT-GTTGT-CTGCGCGTTCGGGAG (55% GC) | TACTC | TTCAC |
| VR1 TACAGTT- | TTGGTTTTCGT-GTTGT-CTGCGCGTTCGGGAG (31 nt) | GATTC | TTCAGtag |
| VR2 TACGCTG- | TTGGTTTTCGT-GTTGT-CTGCGCGTTCGGGAG | GACTT | TTCAGtag |
| Nostoc Punctiforme | | | |
| TR AACAATA- | TTGGTTTTCGT-GTTGT-CTGCGCGTTCGGGA (53% GC) | TGTC | TCTTCA |
| VR1 AGCAATG- | TTGGTTTTCGT-GTTGT-CTGCGCGTTCGGGA (30 nt) | GGAT | TCTTCAGtag |
| VR2 AGCACTC- | TTGGTTTTCGT-GTTGT-CTGCGCGTTCGGGA | GGAT | TCTTCAGtag |
| Nostoc spp. 7120 #2 | | | |
| TR CAACGTA- | --GGTTTTCGG-GTTGT-GGTTGTGCGGGGCA | G | |
| VR GCGCGTG- | --GGTTGTCGG-GTTGT-GGTTGTGCGGGGCA | GGCT | TTCTtag |
| Chlorobium | | | |
| phaeobacterodies | | | |
| TR AACAATA- | -TCGG-TTTTCGT-GTTGT-TCGTCCCA | ATCA | TGCCCGTTTTATGGTGCGGTAA |
| VR1 GGCGTTA- | -TCGG-TTTTCGT-GTTGT-TCGTCCCA | GTCA | TCTTTTGtgaTTATCTGAT |
| VR2 TACGGTT- | -TCGG-TTTTCGT-GTTGT-TCGTCCCA | GTCA | TCTTTTGtgaTTATCTGATAC |
| Pelodictyon | | | |
| phaeoclathratiforme | | | |
| TR AACAATA- | -TTGG-CTTTCGG-GTTGT-CCGTTCCA | ATCAT | GCCCCTTTCGATGCGTGTTAAAG |
| VR GGCAATG- | -TTGG-CTTTCGG-GTTGT-CCGTTCCA | GTCCC | TCTTCCtgaTCTTCTGTCTTTCT |
| Prosthecochloris | | | |
| aestuarii | | | |
| TR ACAACAA- | TTTGGGCTTCCGG-GTTGT-GAG | TACAAAG | TATCGCCAGATGGGGATTGTTTAC |
| VR1 ACGACGT- | TTTGGGCTTCCGC-CTTGT-GAG | GCAGCCT | tagTATCCCTTGGGGTTT |
| VR2 ACGACGA- | TTTGGGCTTCCGC-CTTGT-GAG | GCAGCCT | tagTATCTCTTGGGGTTTTTACCA |

In yet another aspect, the invention provides a method of identifying additional RT coding sequences, IMH and IMH-like sequences, TR sequences, and VR sequences. In one embodiment, the invention provides a method of identifying relevant RT coding sequences by searching sequences for the presence of one or both of a conserved nucleotide binding site motif including amino acid sequences IGXXXSQ or LGXXXSQ, where “X” represents any naturally occurring amino acid. Any suitable methodology for searching sequence information may be used. Non-limiting examples include the searching of protein sequence databases with BLAST or PSI-BLAST.

The invention also provides a method of identifying IMH sequences, said method comprising identifying an RT coding sequence in a genome of an organism, optionally as described above, search the coding strand within about 5 kb of the RT ORF and identify an IMH-like sequence containing an 18-48 nucleotide stretch of adenine-depleted DNA; and
a) use the putative IMH-like sequence to search genome-wide for a closely-related putative IMH and compare the DNA sequences located 5′ to the IMH-like and putative IMH sequences to find homologous TR and VR regions, respectively; or
b) use the sequence of the DNA located 100-350 base-pairs long 5′ to the IMH-like sequence to identify a putative TR, and use all or parts of this TR and IMH-like sequence to search genome-wide for a matching putative VR and IMH sequence.

A potential VR region may be optionally selected for further analysis if present within coding sequence(s) or putative coding sequence(s). A potential TR may be optionally selected based on location in an intergenic region near the RT coding sequence. Of course sequence alignments of potential TR and VR regions may also be used to confirm their operative linkage, especially if sequence differences occur mainly at adenines. As a non-limiting example, the sequences may be more than about 80%, more than about 85%, more than about 90%, or more than about 95% homologous, with the majority of differences being at the locations of the adenines bases in the TR. As an additional option, the identification of the TR or VR sequences may include searching or identification of sequences that are about 100 to about 350 base-pairs long or longer.

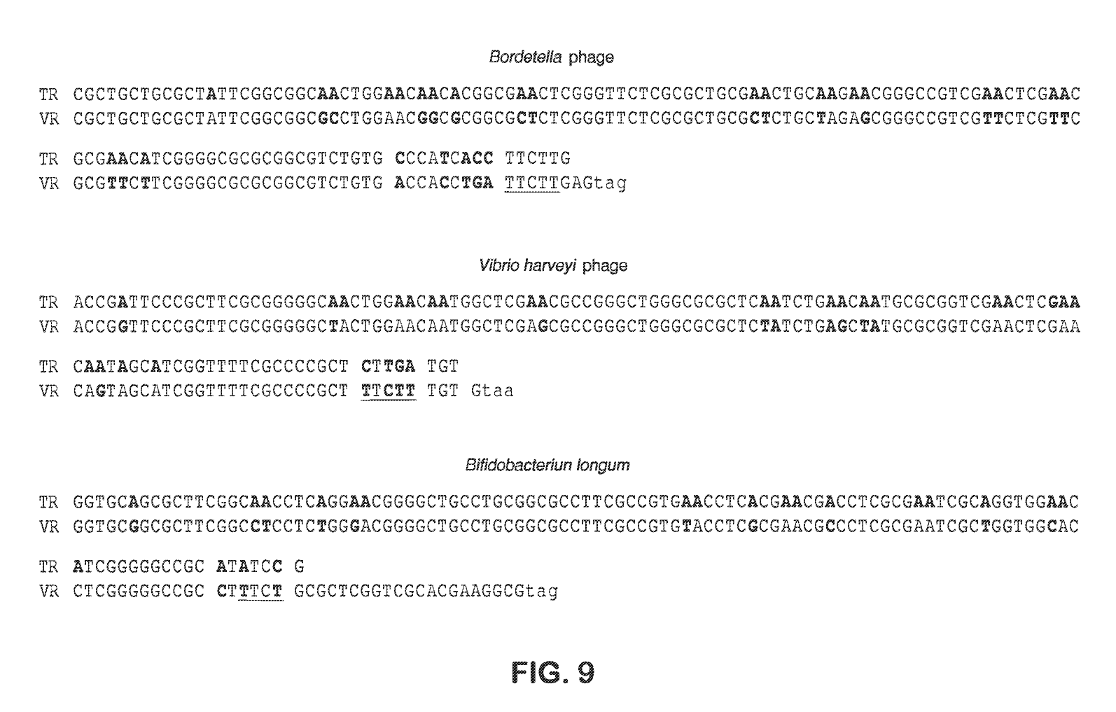

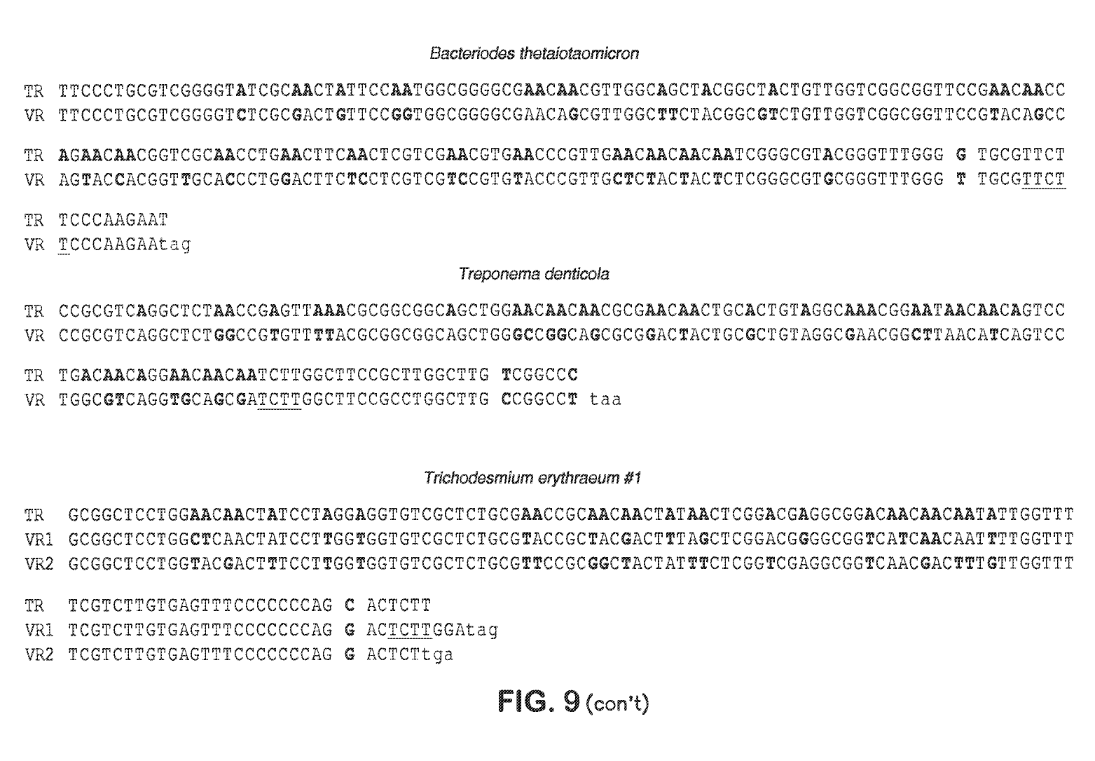

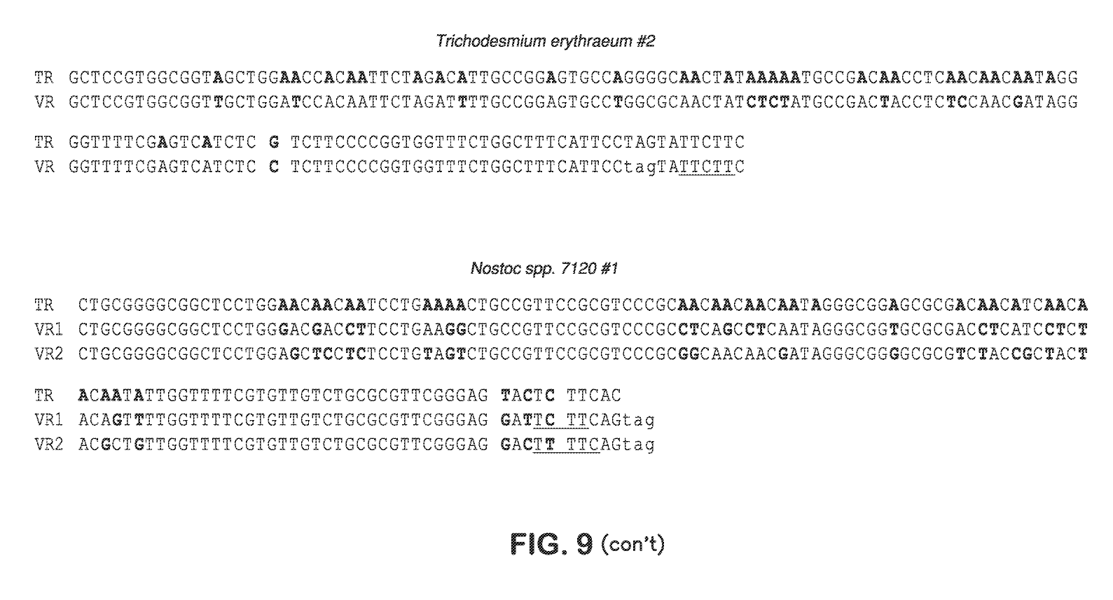

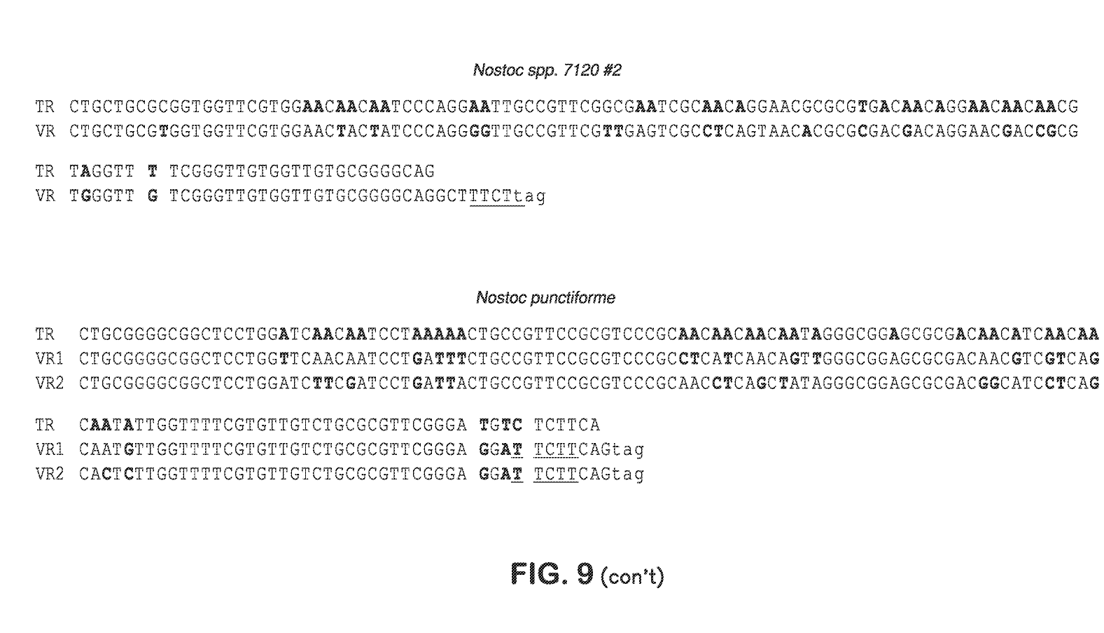

With respect to identifying the IMH-like, or IMH, sequence, searching for a conserved sequence selected from TCGG, TTTTCG, or TTGT at the 3′ ends of possible TR and VR regions may be used. FIG. 9 shows some conserved sequence patterns following the 3′-most nucleotides that vary between TR and VR pairs.

Conserved sequence patterns have been identified as following the 3′-most nucleotides that vary between TR and VR pairs. Comparison of the regions following the VR region (up to or slightly past the position of the VR-containing genes stop codons) revealed several common features, including 1) the length of the regions range from about 18 to about 44 nucleotides (average length of about 38); 2) regions had no or few adenine nucleotides; 3) nearly all (19/23) begin with a TC or TT followed by a sub-region rich in mono- and di-nucleotide runs; 4) all have one or more mismatches near the 3′ end (up to 5 mismatches in a 9 nucleotide stretch); and 5) the majority (13/23) have a TCTT motif and others (5/23) a similar motif near the 3′ end of the region. Thus IMH and IMH-like sequences of the invention may be designed to possess one or more of these features.

The above methods may be in the form of a bioinformatic algorithm to identify DGRs and IMHs. As would be recognized by the skilled person, the above methods may be embodied in the form of a computer readable medium (such as software).

As one alternative, the BPP1 brt protein sequence may be used to search for homologs in the protein database using PSI-BLAST. Brt homologs from previously identified, putative DGRs may be used for a second iteration search, and top hits may be examined further for TR and IMH-like sequences in the vicinity of the RT coding sequence. In some embodiments, genomic regions of about 2000 to 5000 bp upstream and downstream from the RT coding sequence in the genomes of organisms with closely related RT genes may be searched for direct repeats, such as for <4 repeats of >50 nt long. Potential TR and VR regions may be identified if repeats occurred at the 3′-end of an upstream gene and in the intergenic region upstream of the RT gene. Sequence alignment of putative TR and VR regions identified putative DGRs if sequence differences occurred mainly at adenines. The 3′ ends of the putative TR and VR regions may be examined for conserved IMH and IMH-like sequence motifs as described above.

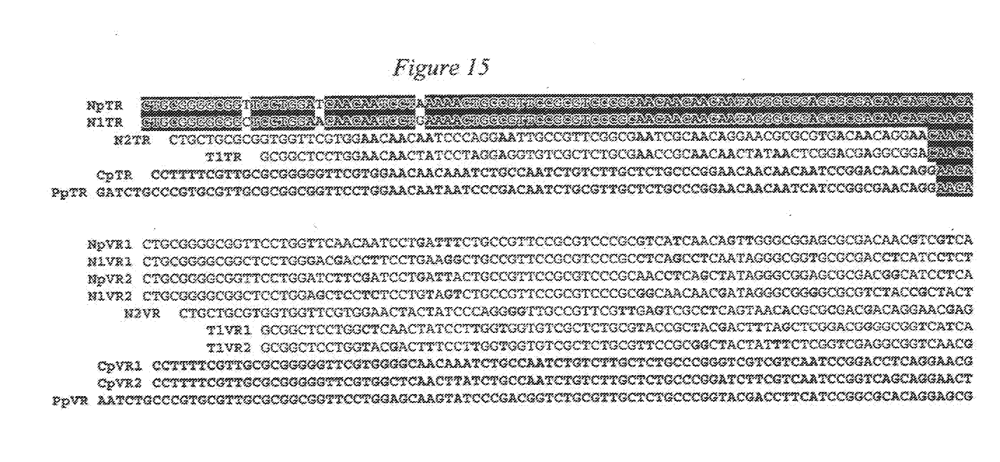

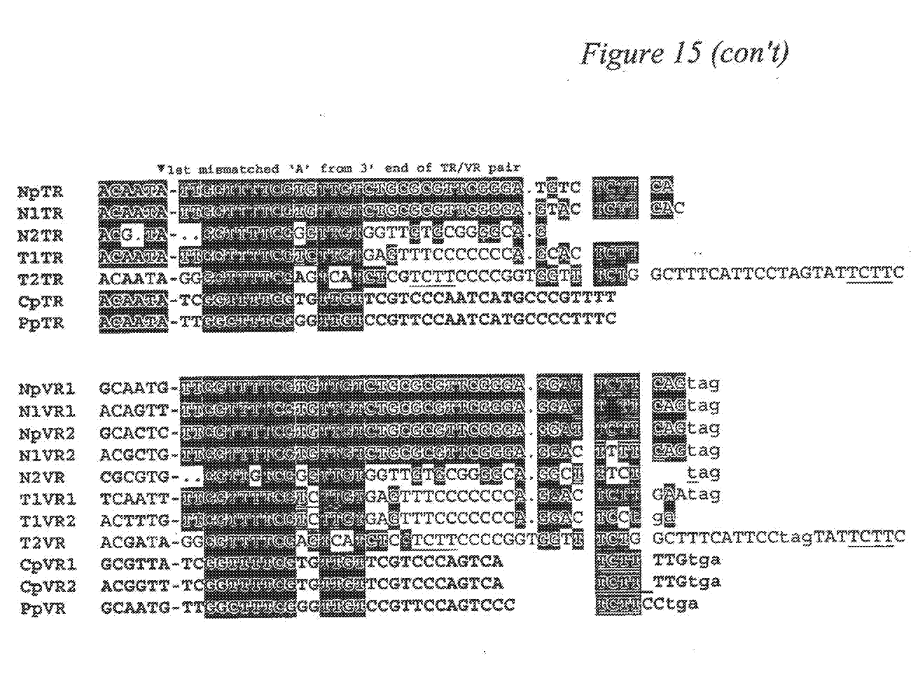

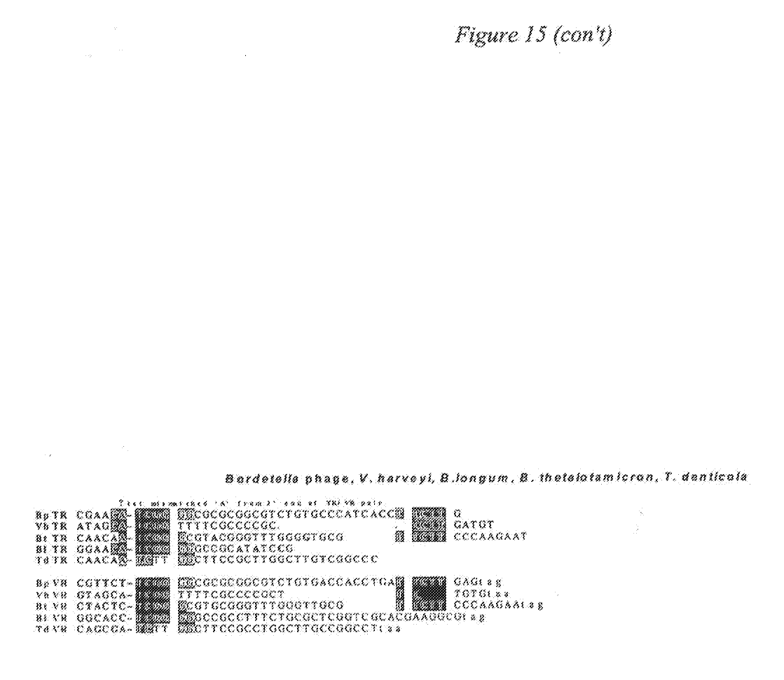

The invention further provides at least two pattern classes derived from alignments of the non-varying 3′ ends of TRs and VRs. Cyanobacterial sequences form a highly similar sub-group, while other TR/VR pairs have conserved sequence motifs at one or both the ends of the regions with dissimilar internal sequences (see FIG. 15). Stop codons were located at variable distances downstream from conserved sequence motifs in each region.

Non-limiting examples of sequences for site-specific mutagenesis according to the invention are those encoding all or part of a binding partner of a target molecule. Non-limiting examples of binding partners include amylin, THF-γ2, adrenomedullin, insulin, VEGF, PDGF, echistatin, human growth hormone, MMP, fibronectin, integrins, calmodulin, selectins, HBV proteins, HBV antigens, HBV core antigens, tryptases, proteases, mast cell protease, Src, Lyn, cyclin D, cyclin D kinase (Cdk), p16^{INK4}, SH2/SH3 domains, SH3 antagonists, ras effector domain, farnesyl transferase, p21^{WAF}, Mdm2, vinculin, components of complement, C3b, C4 binding protein (C4BP), receptors, urokinase receptor, tumor necrosis factor (TNF), TNFα receptor, antibodies (Ab) and monoclonal antibodies (MAb), CTLA4 MAb, interleukins, IL-1, IL-2, IL-3, IL-4, IL-5, IL-6, IL-7, IL-8, IL-9, IL-10, IL-1, IL-12, IL-13, IL-17, interferons, LIF, OSM, CNTF, GCSF, interleukin receptors, IL-1 receptor, c-MpI, erythropoietin (EPO), the EPO receptor, T cell receptor, CD4 receptor, B cell receptor, CD30-L, CD40L, CD27L, leptin, CTLA-4, PF-4, SDF-1, M-CSF, FGF, EGF.

In some embodiments of the invention, the binding partner is a bacteriocin (including a vibriocin, pyocin, or colicin), a bacteriophage protein (including a tail component that determines host specificity), capsid or surface membrane component, a ligand for a cell surface factor or an identified drug or diagnostic target molecule.

In additional embodiments, the binding partner may be part of a fusion protein such that it is produced as a chimeric protein comprising another polypeptide. The other polypeptide member of the fusion protein may be selected from the following non-limiting list: bacteriophage tail fibers, toxins, neurotoxins, antibodies, growth factors, chemokines, cytokines, neural growth factors.

In additional embodiments, the binding partner may be a nucleic acid, part of a nucleic acid molecule, or an aptamer.

As described above, the invention also provides for isolated nucleic acid molecules derived from naturally occurring sequences. Such an isolated nucleic acid molecule may be described as comprising a donor template region (TR) and a variable region (VR) wherein said TR is a template sequence operably linked to said VR in order to direct site specific mutagenesis of said VR. Preferably, the molecule is from a bacteriophage but not from Bvg+ tropic phage-1 (BPP-1), Bvg⁻ tropic phage-1 (BMP-1), or Bvg indiscriminate phage-1.

The nucleic acid molecules of the invention may be part of a vector or a pair of vectors that is/are introduced into cells that permit site-specific mutagenesis of the VR and/or support replication of the molecules. Non-limiting examples of vectors include plasmids and virus based vectors, including vectors for phage display that may be used to express a diversified VR sequence. Other non-limiting embodiments are vectors containing VR sequences that have been subjected to the methods of the instant invention and then removed from an operably linked TR, including by preventing the expression of TR, so as to produce without further diversification quantities of the VR-encoded protein for uses including as a diagnostic, prognostic, or therapeutic product.

The instant invention also provides for a “diversified collection” of more than one VR sequence, per se or in the context of a vector, wherein at least two of the VR sequences differ from each other in sequence. In some embodiments, the difference in sequence results in the encoding of a different polypeptide by the VR sequence, but the difference may also be silent or synonymous (different codon encoding the same amino acid) and optionally used in cases where codon optimization is needed to improve expression of the encoded polypeptide. A “diverse collection” may also be referred to as a library or a plurality of VR sequences, per se or in the context of a vector. Thus the invention also provides a plurality or library of nucleic acid molecules as described herein. The plurality or library of molecules may include those wherein the VR has undergone diversification directed by the operably linked TR.

Non-limiting examples of cells that contain the nucleic acids of the invention include bacterial cells that support site-specific mutagenesis of bacteriophages as described herein or eukaryotic cells of any species origin that support mutagenesis and/or production and processing of recombinant mutagenized protein. In some embodiments, yeast or fungal cells may be used. In other embodiments, higher eukaryotic cells may be used.

### Diversity-Generating Retroelements

Using a Bordetella bacteriophage DGR as a model, we demonstrated that homing occurs through a TR-containing RNA intermediate and is RecA-independent. Marker transfer studies showed that cDNA integration at the 3′ end of VR occurs within a (G/C)₁₄ element, and deletion analysis demonstrated that the reaction was independent of 5′-end cDNA integration. cDNA integration at the 5′ end of VR required only short stretches of sequence homology. We have demonstrated that homing occurs through a target DNA-primed reverse transcription (TPRT) mechanism that precisely regenerated target sequences. This non-proliferative, “copy and replace” mechanism enabled repeated rounds of protein diversification and optimization of ligand-receptor interactions.

Based on the requirement for an RT activity, homing was hypothesized to occur through an RNA intermediate (Liu, M. et al., Science, 295:2091-2094 (2002); Medhekar, B. and Miller, J. F., Curr. Opin. Microbiol., 10:388-395 (2007)). Using a plasmid donor system expressing the BPP-1 atd, TR, and brt loci in trans (Xu et al., unpublished data), we have shown that TR accommodated insertions of heterologous sequences and that adenine mutagenesis occurred efficiently during transfer of inserted sequences to VR. By engineering a self-splicing group I intron into TR, we provided conclusive evidence that DGR homing occurs via an RNA intermediate and was RT-dependent. We also identified regions of the TR-containing RNA transcript that are important for homing. Interestingly, although VR and TR share significant homology, homing was found to be RecA-independent, preferably a marker coconversion assay showed that cDNA initiation occurs within the (G/C)₁₄ element of VR, and further analysis demonstrated that cDNA initiation does not have VR sequences upstream of the (G/C)₁₄ region, but has IMH. cDNA integration upstream of the (G/C)₁₄ element had short stretches of homology between VR and cDNA, and was otherwise sequence-independent. On the basis of these and other results, we conclusively demonstrated that DGR homing initiated via a specialized target DNA-primed reverse transcription (TPRT) mechanism. This approach rendered mechanistic insights into the non-proliferative, “copy and replace” pathway of DGR homing, and accounted for the ability of the DGR to regenerate target sequences in a manner that enabled repeated rounds of homing and VR diversification. Our results provided new approaches for DGR-based genetic engineering.

### Multiple Sites in TR can Tolerate Heterologous Sequence Insertions

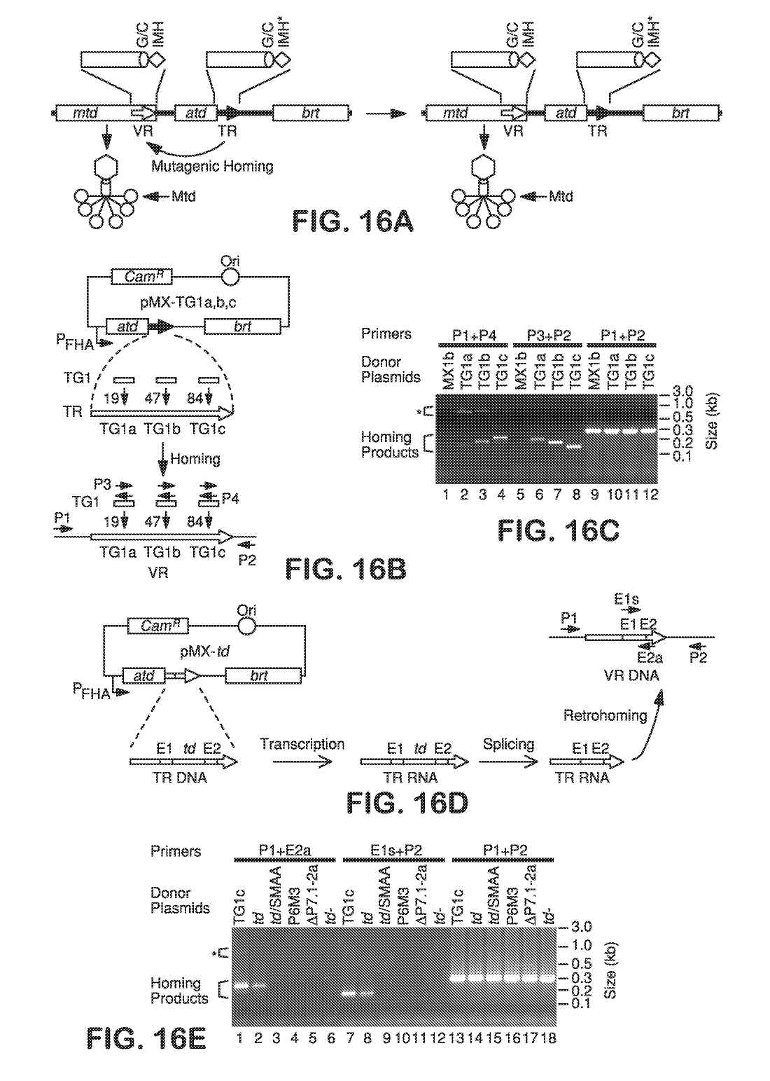

We initially set out to tag the BPP-1 TR with a self-splicing group I intron, which could then be used to determine whether DGR homing occurs through an RNA intermediate. Intron tagging is a classic method for verifying retrotransposition of mobile genetic elements (Boeke, J. D. et al., Cell, 40:491-500 (1985); Cousineau, B. et al., Cell, 94:451-462 (1998); Guo, H. et al., Science, 289:452-457 (2000); Moran, J. V. et al., Cell, 87:917-927 (1996)). As a prerequisite, the ability of TR to tolerate DNA insertions was first assessed. We inserted a 36 bp fragment at three different positions in TR on plasmid pMX1b, which expressed atd, TR, and brt from the BvgAS activatedjhaB promoter (FIG. 16B) (Jacob-Dubuisson, F. et al., Microbiology, 146:1211-1221 (2000)). The 36 bp fragment contained the ligated exons of the phage T4 td group I intron, flanked by SalI sites (Cousineau, B. et al., Cell, 94:451-462 (1998); Guo, H. et al., Science, 289:452-457 (2000)). The resulting plasmids pMX-TG1a, pMX-TGIb, and pMX-TG1c, and the parental plasmid pMX1b, were transformed into B. bronchiseptica strain RB50. Transformed cells were induced to express pertactin, the BPP-1 phage receptor, and to activate the fhaB promoter. Following single-cycle lytic infection with a derivative of BPP-1 containing null mutations in TR and brt (BPP-1d), a homing assay was performed on DNA isolated from progeny phages. Although BPP-1d is defective for tropism switching and DGR activity, it was efficiently complemented by pMX1b in trans. Using the 36 bp insert as a tag (TG1), we devised a PCR-based assay to detect TR-derived TG1's transferred to VR as a result of homing. As shown in FIG. 16B, three sets of primers were used: primers P1/P4 amplified TG1's transferred to VR along with upstream sequences; primers P2/P3 amplified TG1's transferred to VR along with downstream sequences; and primers P1/P2 amplified VR and flanking sequences to confirm equal input of phage DNA in PCR reactions.

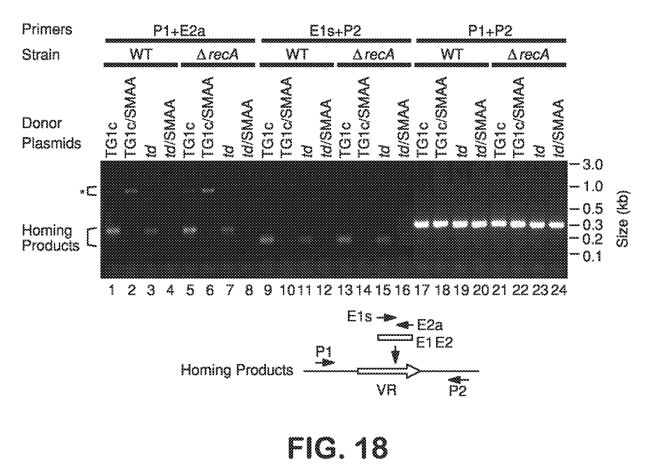

Using primer pairs P1/P4, and P2/P3, we detected PCR products of predicted sizes resulting from complementation with all of the constructs containing TG1 (FIG. 16C). No products were detected with the same primers following complementation with pMX1b, which lacked TG1, or with the brt-deficient plasmid pMX-TG1c/SMAA (data not shown; FIG. 18). Sequence analysis of PCR products demonstrated adenine mutagenesis of TG1 inserts as well as flanking VR sequences (FIGS. 16-18 and data not shown), confirming that they were derived from homing events. Of the three TR insertion sites, position 84 appeared to retain the highest homing activity. These results demonstrate that TR tolerated heterologous sequence insertions and that inserted sequences were transferred to VR and were subject to adenine mutagenesis. The PCR assay shown in FIG. 16B provided a sensitive and specific means to detect DGR homing products in a manner that was independent of tropism switching or phage infectivity.

DGR Homing Occurred through an RNA Intermediate

To demonstrate that DGR homing occurred through a TR-containing RNA intermediate, we used a modified group I intron, tdΔ1-3, which lacked most of the intron ORF but retained self-splicing activity (Cousineau, B. et al., Cell, 94:451-462 (1998); Guo, H. et al., Science, 289:452-457 (2000)). Plasmid pMX-td contains the modified td intron inserted into TR at position 84 (FIG. 16D). Following intron splicing, pMX-td transcripts only retain ligated exons. Some VRs that had undergone mutagenic homing acquired precisely ligated exons from the intron-bearing TR.

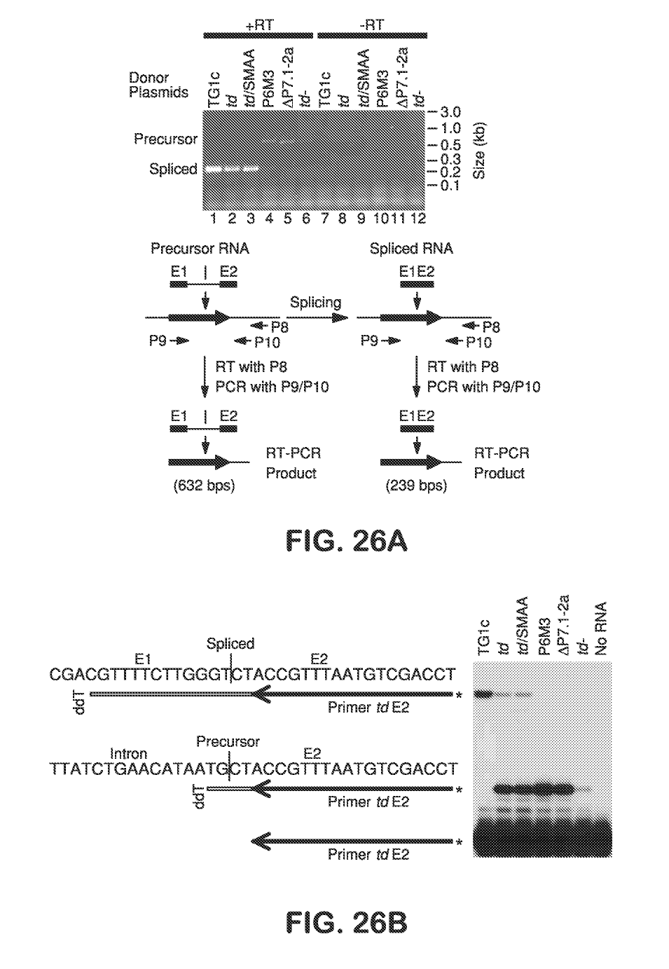

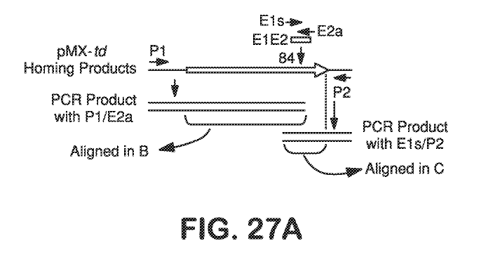

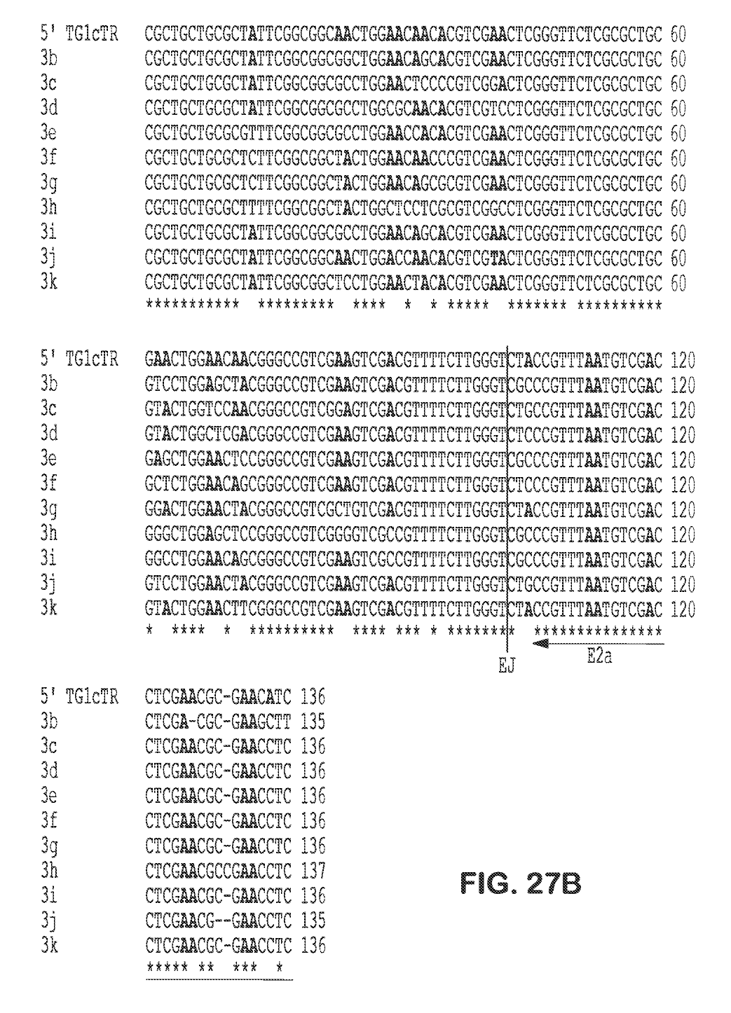

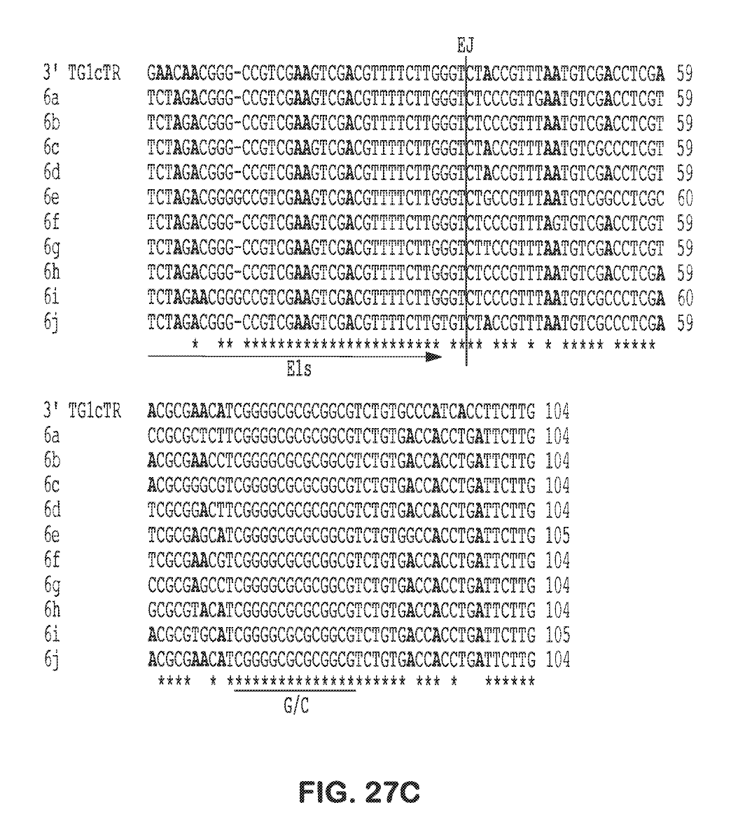

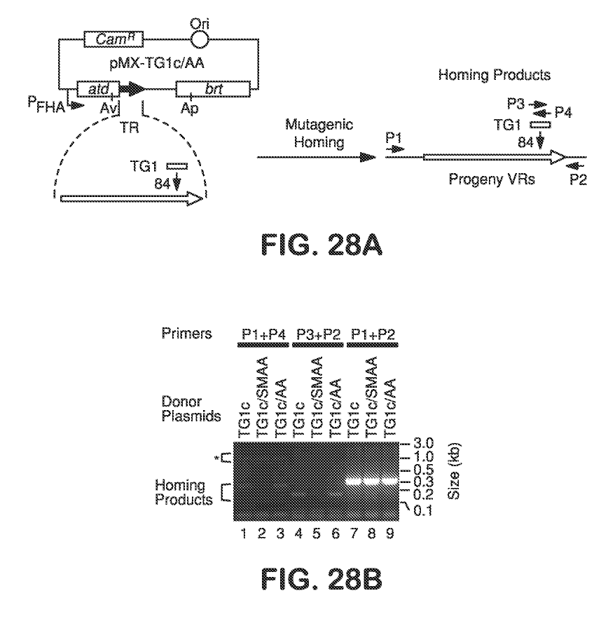

The td intron was verified to be capable of RNA splicing in Bordetella through both reverse transcription-polymerase chain reaction (RT-PCR) and primer extension-termination assays (FIG. 26) (Zhang, A. et al., RNA, 1:783-793 (1995)). No adenine substitutions were detected in RT-PCR products generated from spliced transcripts, showing that nucleotide alterations did not occur at the RNA level. We next determined that the intron-tagged TR was capable of homing and characterized the homing products. RB50 cells transformed with pMX-td or control plasmids were infected with BPP-1d, and DNA isolated from progeny phages was subjected to PCR analysis. Two td exon primers were used to detect homing products: E1s was a sense-strand primer annealing to exon 1 (E1), and E2a was an antisense-strand primer annealing to exon 2 (E2) (FIG. 16D). With primers P1/E2a, and primers E1s/P2, we observed PCR products generated with Pmx-td that were identical in size to those from the positive control, pMX-TG1c, which contains ligated exons inserted at TR position 84 (FIG. 16E). This showed that spliced transcripts had been used for homing. The lower amount of homing products observed with pMX-td compared to pMX-TG1c correlated with the observation that the spliced RNA product produced by pMX-td corresponded to 18% of the pMX-TG1c transcripts containing ligated exons (FIG. 26B). Characterization of homing products confirmed that the td intron was precisely excised, and that adenine mutagenesis occurred in the ligated exons and flanking sequences (FIG. 28).

Consistent with a retrohoming process, detection of ligated-exon products required a functional td intron, as splicing-defective mutants (P6M3 and ΔP7.1-2a; FIG. 26) (Mohr, G. et al., Cell, 69:483-494 (1992)), and a construct with an inverted intron (td−), failed to generate homing products (FIG. 16E). We did not detect transfer of intron sequences from TR to VR, and this was true for functional as well as nonfunctional derivatives of the td intron. The failure to detect products containing unspliced or unsplicible introns was due to a size limitation for heterologous sequences inserted into TR at position 84. TR can tolerate insertions of about 200 bp at this site (Tse et al., unpublished), and this limit was exceeded by inserts containing splicing-competent (429 bp) or defective (397-429 bp) td introns. As expected, transmission of ligated exons to VR was RT-dependent, as an RT-deficient mutant (td/SMAA) failed to support detectable homing. Taken together, our results demonstrated that DGR homing is a retrotransposition process which occurred via a TR-containing RNA intermediate.

### Regions of the RNA Transcript Important for Homing

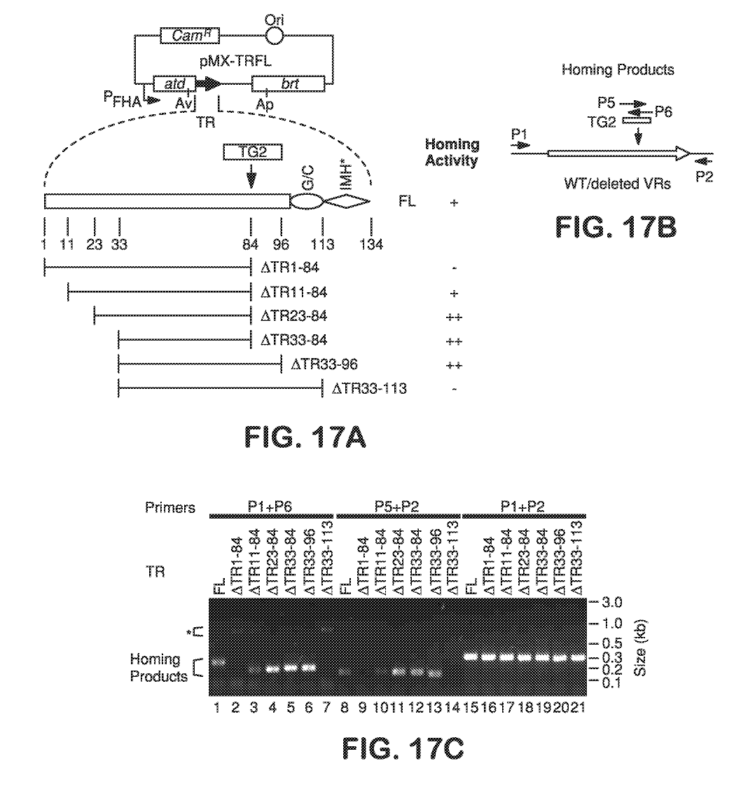

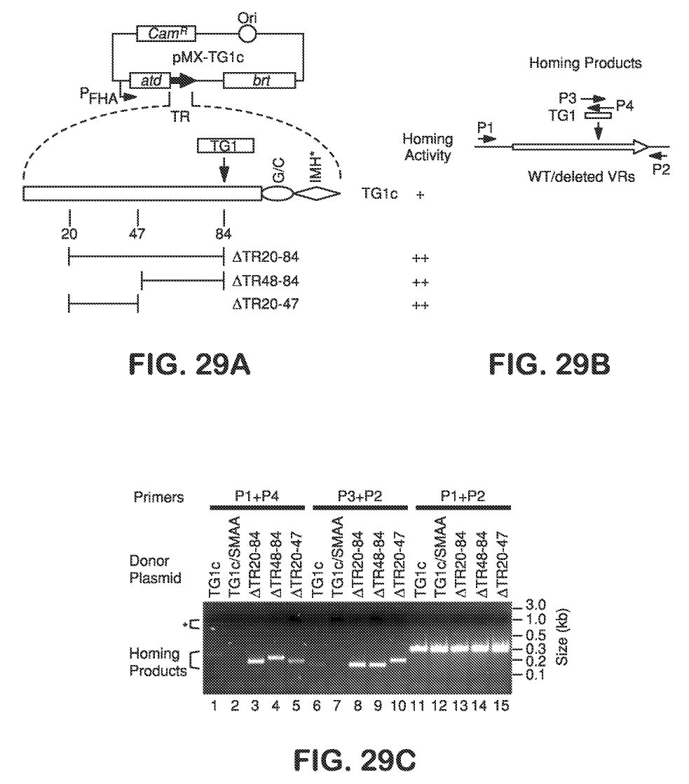

To identify sequence requirements for the RNA intermediate involved in homing, we constructed a series of donor constructs containing deletions within TR and a 32 bp tag (TG2) at the deletion junctions (FIG. 17A). Using PCR-based homing assays with primers P1/P6 and P5/P2, we discovered that most of TR is dispensable (FIG. 17). Preferably ˜10 bp at the 5′ end, and ˜38 bp at the 3′ end are used for homing. The 3′ sequence requirements correspond almost entirely to the (G/C)₁₄ and IMH* elements. Interestingly, deletion constructs ΔTR23-84, ΔTR33-84, and ΔTR33-96 consistently generated a higher abundance of homing products than the donor with a full-length TR (FL in FIG. 17C), indicating that shorter TRs supported more efficient homing. Consistent with these results, TR sequences between the TG1a and TG1c insertions (positions 20 to 84) were found to be dispensable, and their deletion increased DGR homing activity (FIG. 29). Although 10 bp were sufficient for homing, optimal activity required 10-19 bp at the 5′ end of TR. Sequence analysis showed that PCR products from functional TR deletion derivatives underwent adenine mutagenesis, confirming that they were generated by mutagenic homing (data not shown).

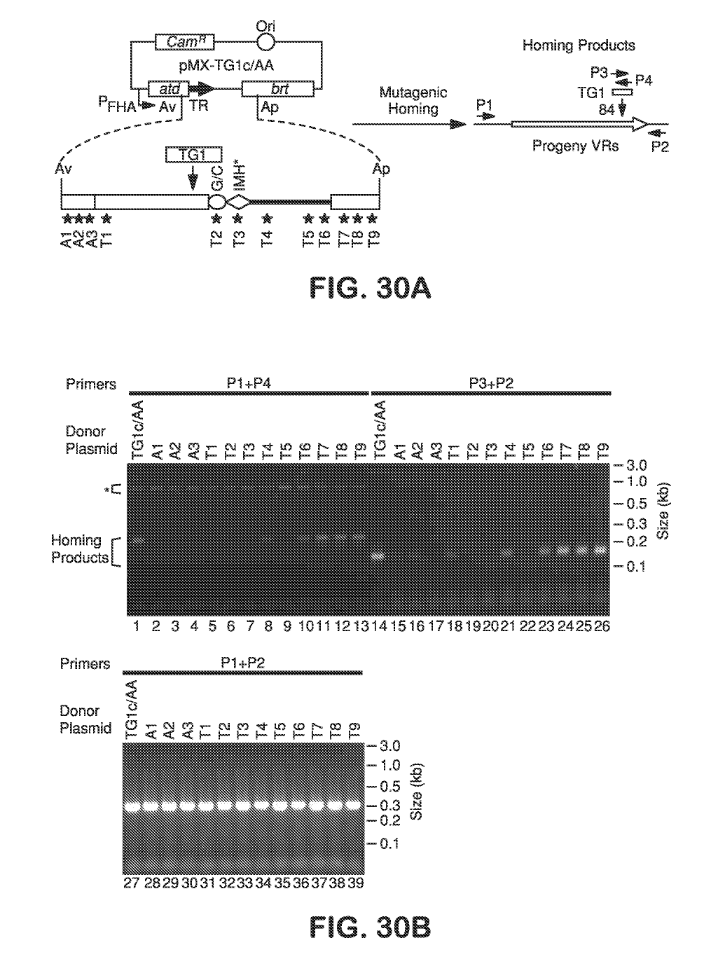

Preferably there are no mutations at positions 8-11 of TR, and in the (G/C)₁₄ and IMH* elements (FIG. 30). Furthermore, silent mutations at the 3′ end of atd and a substitution in the intergenic region between TR and brt (mutation T5 in FIG. 30) also significantly decreased homing efficiency. These results demonstrated that internal sequences were largely dispensable, and TR function preferably had the ends of the repeat and flanking upstream and downstream sequences. These were important for the integrity of an RNA structure important for homing.

### Retrohoming was RecA Independent

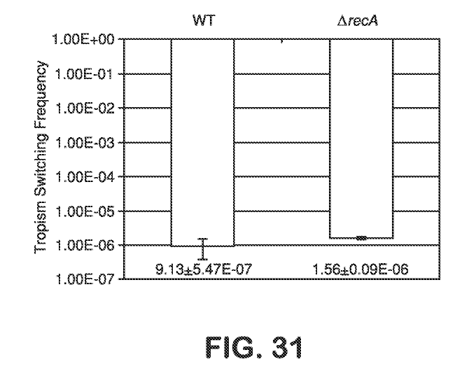

RecA is involved in repairing UV-induced DNA damage and homologous recombination (Courcelle, J. and Hanawalt, P. C., Annu. Rev. Genet, 37:611-646 (2003)). Considering the extent of TR/VR homology, it was important to evaluate the role of RecA in DGR homing. We generated a B. bronchiseptica RB50ΔrecA strain by allelic exchange. The recA knockout was verified by PCR, sequencing, and loss of RecA-dependent DNA repair in a UV-sensitivity assay (data not shown). We analyzed the ability of donor plasmids pMX-TG1c, pMX-td, and their RT-deficient mutants to complement BPP-1d in wild-type RB50 and its isogenic recA-deficient derivative (FIG. 18). The relative yields of homing products generated from DGR-competent plasmids in wild-type and recA mutant strains were indistinguishable, indicating that DGR retrotransposition was RecA-independent. Consistent with these results, tropism switching by BPP-1d was also found to be RecA-independent (FIG. 31).

### Marker Coconversion Boundaries

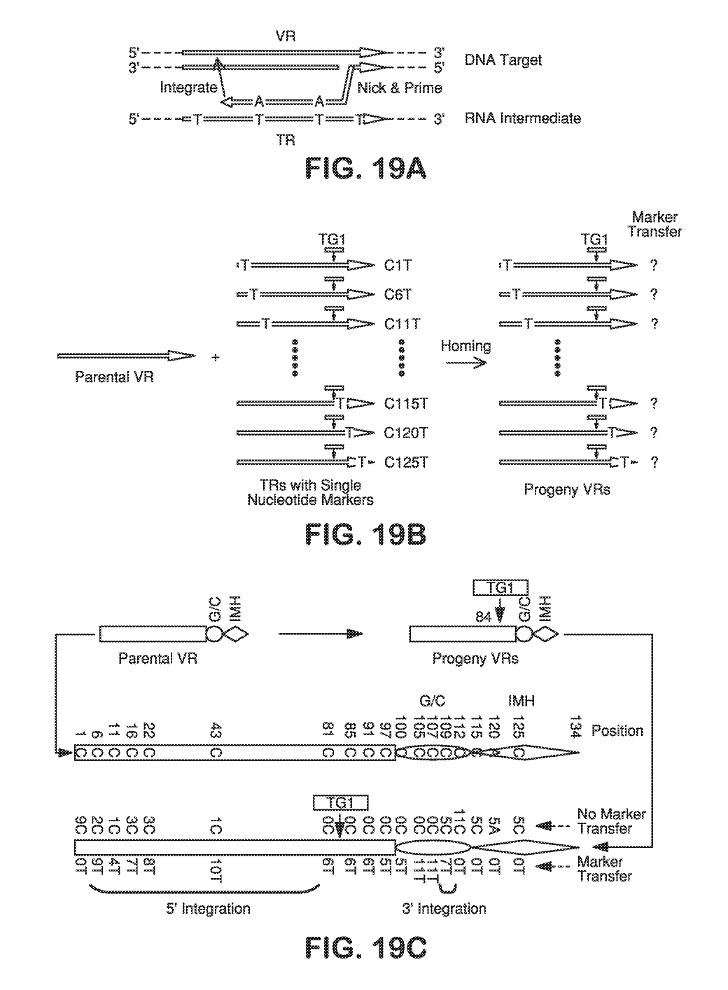

As DGR homing occurred through an RNA intermediate and was RecA-independent, we predicted that cDNA integration at the 3′ end of VR might occur through a TPRT reaction as opposed to homologous recombination following cDNA synthesis. To identify potential priming site(s) for TPRT, we determined that there was a specific boundary at the 3′ end of TR for sequence transfer to VR (FIG. 19A). Such a boundary indicated a site at which cDNA synthesis initiated. We also determined the boundary at the 5′ end of TR for sequence transferin and defined a cDNA integration site at the 5′ end of VR (FIG. 19A). We generated a series of donor constructs with single C to T substitutions at multiple positions in TR, and inserted TG1 at position 84 to facilitate PCR homing assays (FIG. 19B). C to T substitutions were chosen to minimize disruption of potential RNA structures since G residues can form both G-C base pairs and G-U wobble pairs, and single markers were introduced into individual constructs to minimize disruption of homology. Since thymidine residues are not altered during DGR homing, marker coconversion assays allowed a precise mapping of VR sequences that have been acquired from TR. From the boundaries of marker coconversion, sites of cDNA initiation at the 3′ end and integration at the 5′ end of VR were inferred.

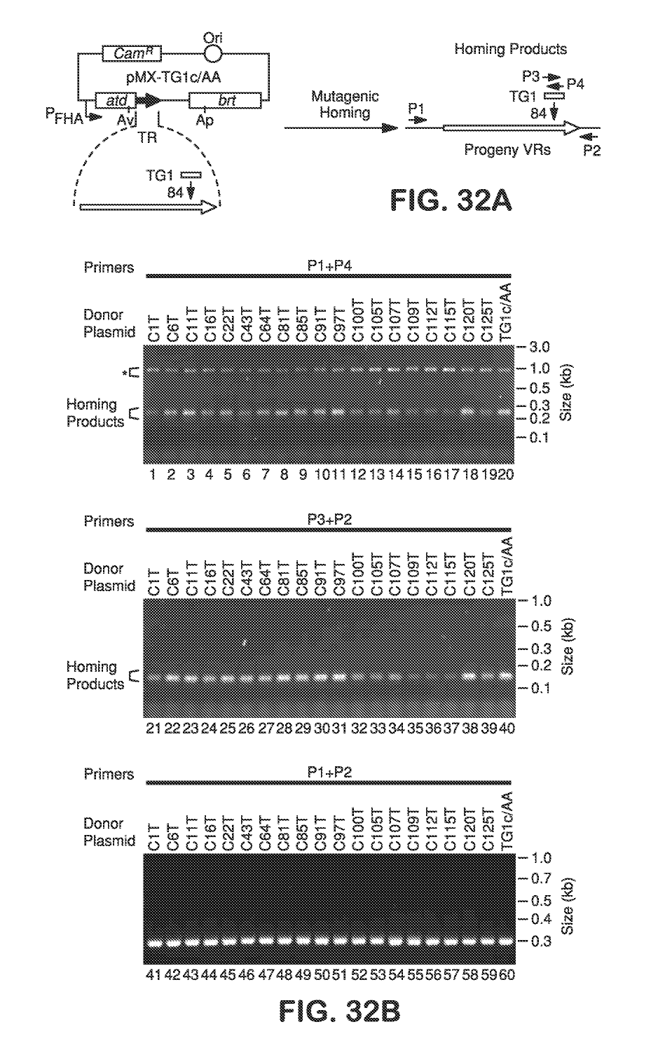

Nineteen donor constructs, with C to T substitutions at TR positions upstream or downstream of the TG1 insertion, were generated and tested for homing into the VR of BPP-1d (FIG. 19B; FIG. 32). Although some substitutions in the (G/C)₁₄ and IMH* elements slightly decreased homing activity, all constructs supported sufficient homing to allow marker coconversion analysis. DGR homing products were cloned and sequences were determined for multiple independent clones derived from each donor construct. As summarized in FIG. 19C, marker coconversion downstream of TG1 occurred with 100% efficiency at positions 85 to 107. At position 109, 5 clones showed marker transfer while 7 did not. No marker transfer occurred at position 112 or at positions further downstream. These data identified a boundary for marker transfer from TR to VR, located within the (G/C)₁₄ element between positions 107 and 112. This coconversion boundary represented sites for cDNA initiation during homing. The heterogeneity observed at position 109 indicated that initiation occurred at multiple sites between positions 107 and 112.

Substitutions in the 5′ region of TR displayed a more diffuse pattern of marker transfer. The marker immediately upstream of TG1 (C81T) was transmitted with 100% efficiency. This was expected, since PCR homing assays selected for TG1 transfer with nearby sequences subjected to co-selection. At greater distances upstream of the tag, coconversion was observed for the majority of clones, with the exception of the marker at position 1. The observation that markers at positions 6, 11, 16, 22 and 43 were partially transferred indicated that cDNA integration occurred before extending to the 5′ end of TR.

Sequences from PCR assays displayed adenine mutagenesis, confirming their assignment as DGR homing products.

cDNA Integration at the 3′ End of VR Was Independent of 5′-End Integration

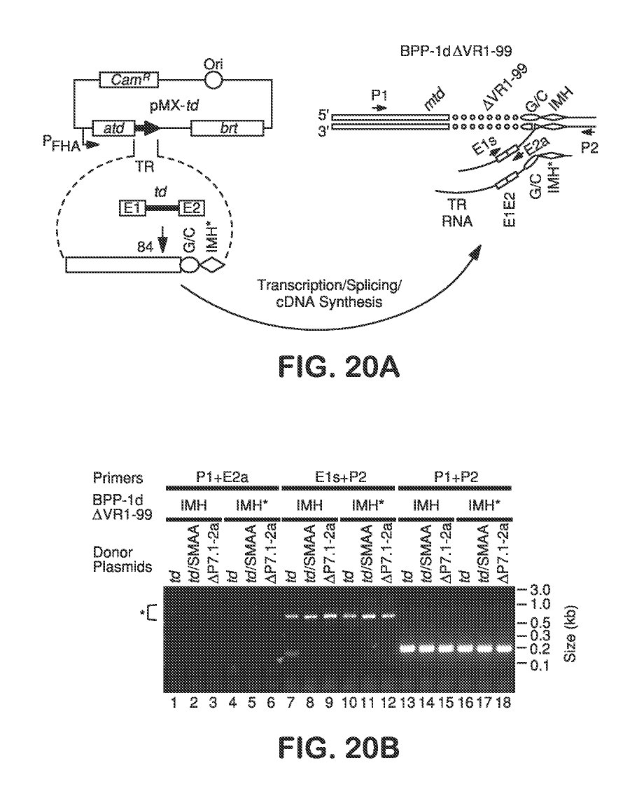

Results from marker coconversion assays indicated that homing took place in a sequential manner, with cDNA integration at the 3′ end of VR occurring first through a TPRT reaction, followed by cDNA integration at the 5′ end mediated by VR/TR homology. We determined that cDNA integration at the 3′ end of VR occurred independently of 5′-end integration by deleting all VR sequences upstream of the (G/C)₁₄ and IMH elements, creating prophage BPP-1dΔVR1-99 which lacked the first 99 bp of VR (FIG. 20A). The VR1-99 deletion was predicted to eliminate cDNA integration at the 5′ end, preventing complete homing but allowing 3′-end cDNA integration. As a negative control, we replaced IMH with IMH* to generate prophage BPP-1dΔVR1-991MH*.

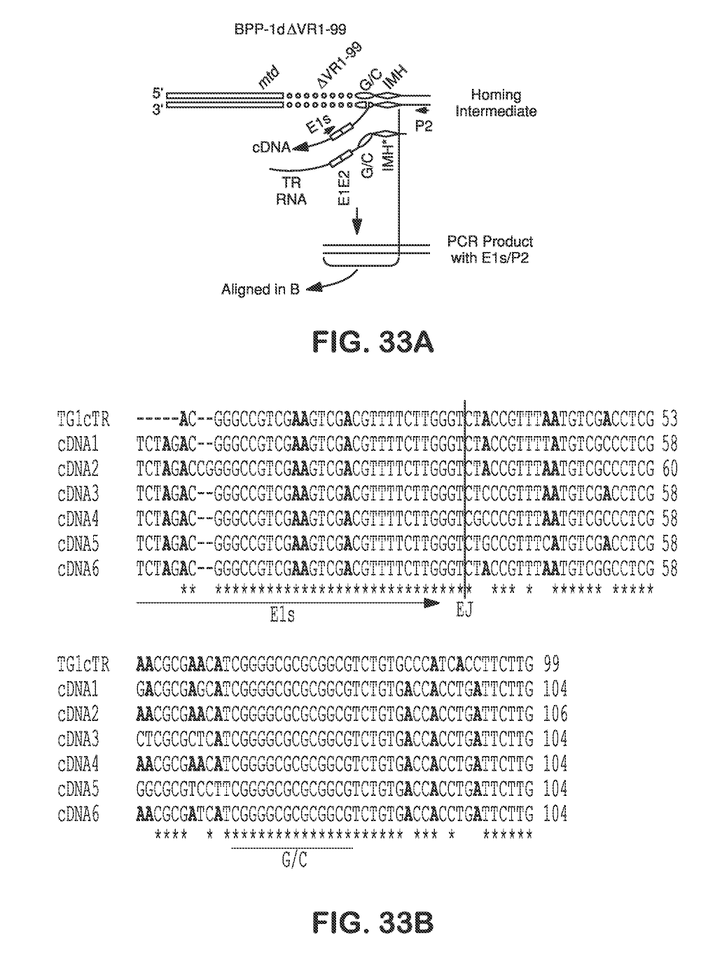

The td intron-tagged pMX-td (FIG. 16D), and negative control derivatives pMX-td/SMAA (RT-deficient) and pMX-td/ΔP7.1-2a (splicing-defective), were used to complement mutant prophage in homing assays. By using an intron-tagged TR, we identified true cDNA products since they acquired ligated exons. With primers E1s and P2, a specific product of the expected size (167 bp) was detected in DNA isolated from pMX-td transformed BPP-1dΔVR1-99 lysogens (FIG. 20B, lane 7). This product was not detected in samples from the same lysogen complemented with Brt-deficient or splicing-defective donor plasmids, nor in samples from BPP-1dΔVR1-99IMH* lysogens transformed with pMX-td. These results showed that the 167 bp product was the result of a Brt- and IMH-dependent retrohoming reaction. This was confirmed by sequence analysis, which demonstrated precise exon ligation and adenine mutagenesis (FIG. 33). In contrast, PCR assays with primers P1 and E2a did not detect a specific product (FIG. 20B, lanes 1-6). This was not due to primer failure, since this primer pair efficiently amplified a product of correct size (240 bp) in progeny phages when pMX-TG1c transformants were infected with BPP-1d (data not shown; FIGS. 16 E and 18). The product detected in FIG. 20B (lane 7, arrow) represented a cDNA intermediate “trapped” in the homing reaction, as depicted in FIG. 20A.

Taken together, these results demonstrated that cDNA integration at the 3′ end of VR can occur independently of 5′-end integration, demonstrating that DGR homing initiated through a TPRT mechanism. cDNA initiation at the 3′ end of VR had IMH, but was independent of VR sequences or VR/TR homology upstream of the (G/C)₁₄ element.

cDNA Integration at the 5′ End of VR

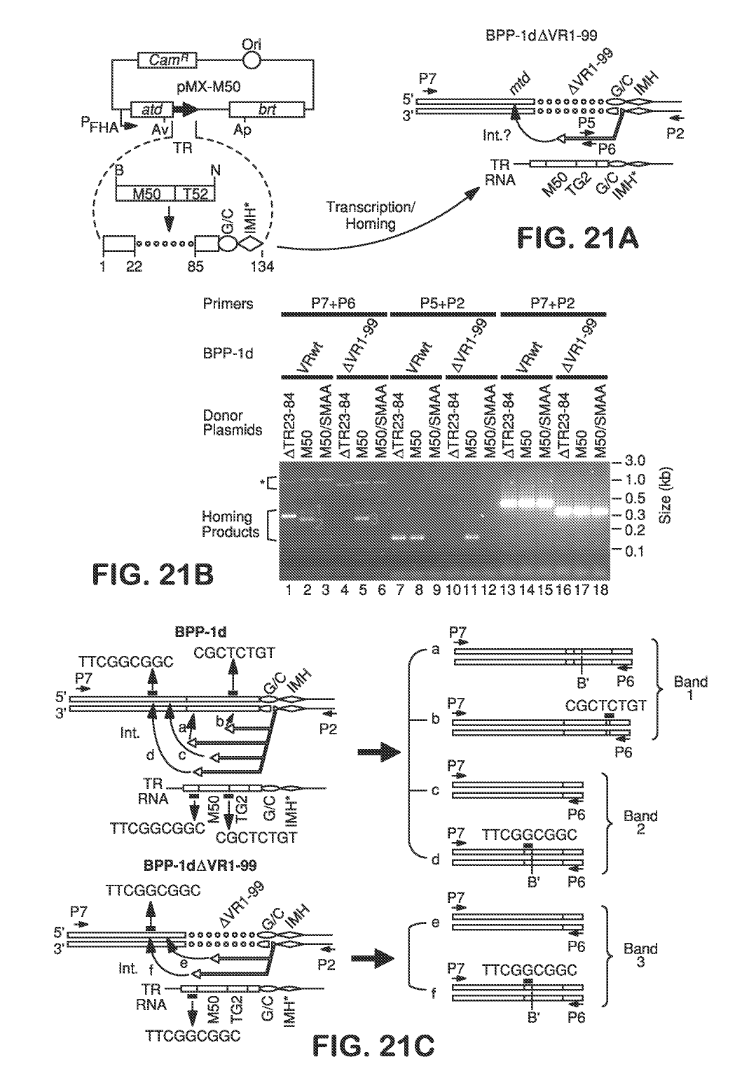

We showed that cDNA integration at the 5′ end of VR had TR/VR homology upstream of the (G/C)₁₄ and IMH elements, as opposed to specific sequences. We determined that the homing defect resulting from the VR1-99 deletion was rescued by inserting a 50 bp segment of mtd (M50), derived from sequences upstream of VR, into the TR of plasmid pMX-ΔTR23-84 (FIG. 17A). The resulting plasmid, pMX-M50 (FIG. 21A), and control constructs pMX-M50/SMAA (Brt-deficient) and pMX-ΔTR23-84 (lacking the M50 insert) were evaluated for their ability to support homing in BPP-1d and BPP-1dΔVR1-99 lysogens.

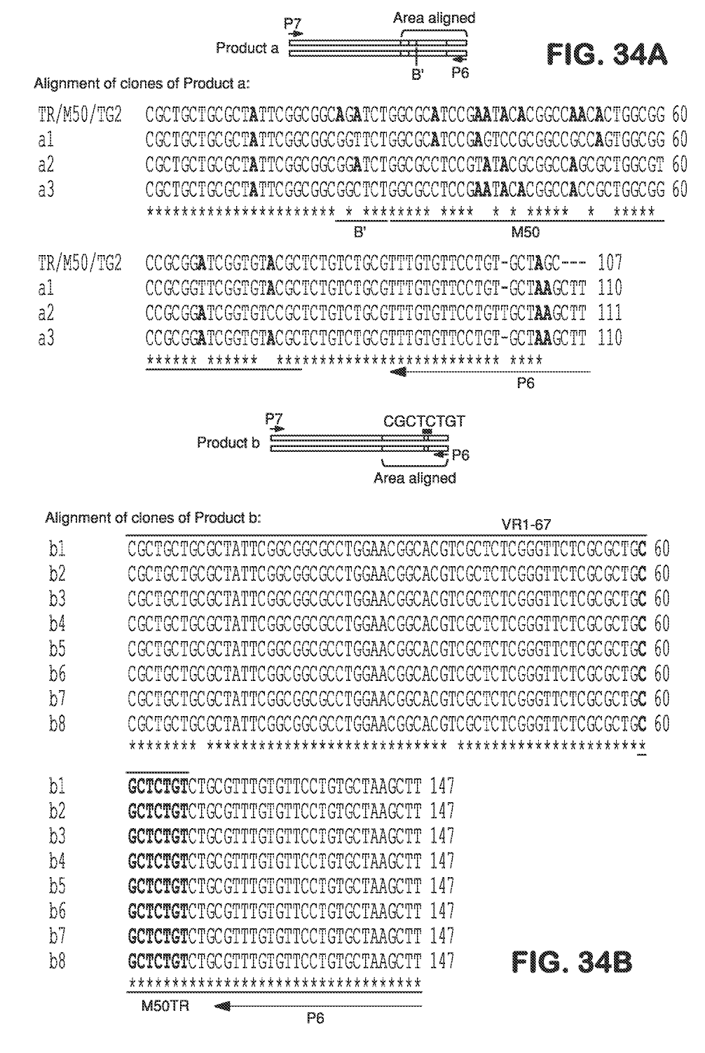

As shown in FIG. 21B, pMX-ΔTR23-84 and pMX-M50 supported homing into the VR of BPP-1d with similar efficiencies, while the Brt-deficient donor did not. Interestingly, pMX-M50 yielded two PCR products in the expected size range with primers P7/P6 (FIG. 21B, lane 2), both of which were cloned and characterized. We suspected that band 1 corresponded to cDNA integration within the first 22 bp region of VR mediated by TR/VR homology, generating a slightly larger product than observed with pMX-ΔTR23-84 due to the M50 insert (FIG. 21C, a). Out of 15 clones analyzed, 3 had this structure, and adenine mutagenesis patterns showed they were the products of mutagenic homing events (FIG. 34A). A majority (8/15) of the clones obtained from band 1, however, corresponded to cDNA integration at VR positions 60-67 via a homologous 8 nucleotide (nt) sequence located at the junction of the M50 insert and the TG2 tag (FIG. 21C, b; FIG. 34B). These were derived from true homing products, as they were the major species from band 1, which was not detected with the Brt-deficient mutant pMX-M50/SMAA (FIG. 21, lane 2 vs. 3). Integration occurred through cDNA template switching from the engineered TR to VR within the 8 nt homologous sequence. In addition, 4 minor species of clones were detected, each of which was also accounted for by cDNA integration through template switching between short stretches (4-13 bp) of sequence homology (data not shown).

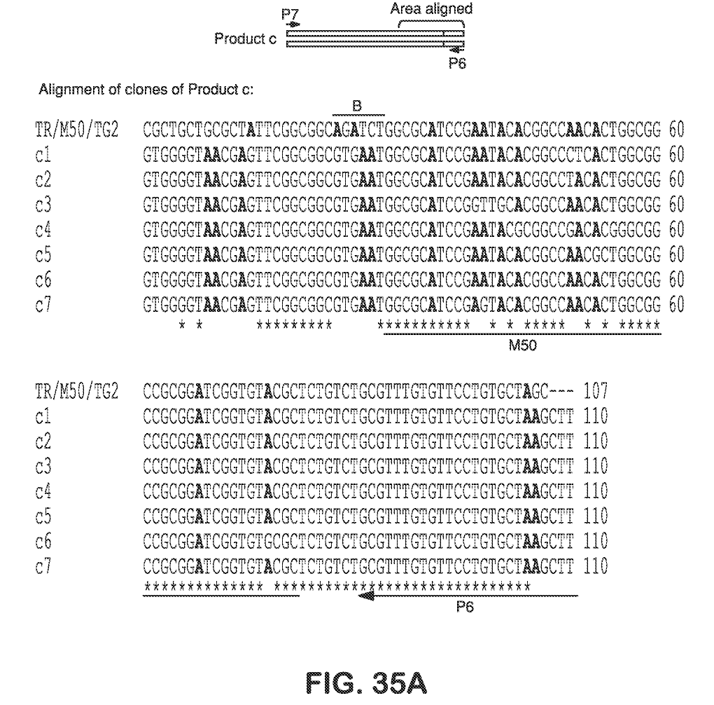

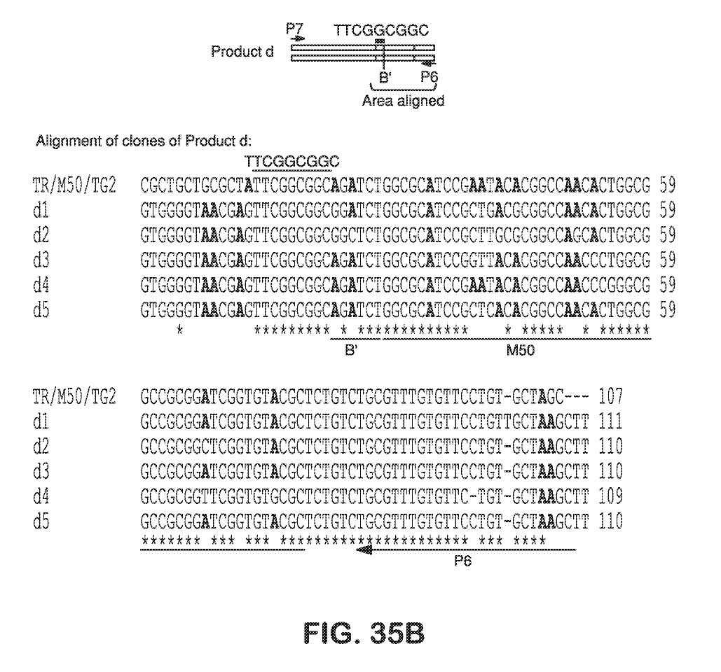

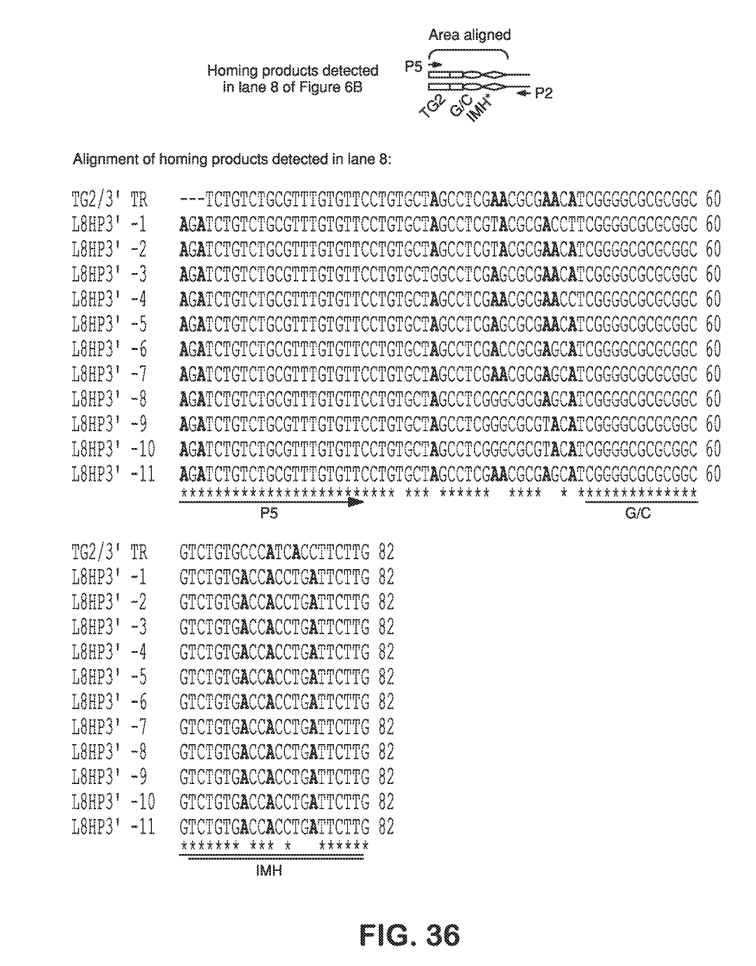

Sequence analysis of clones from band 2 revealed two products of the same size. One (FIG. 21C; FIG. 35A) corresponded to 5′ cDNA integration through the M50 insert in TR. The other product (FIG. 21C, d; FIG. 35B) corresponded to cDNA integration via yet another short stretch of sequence homology (9 nt), located 6 bp upstream of the M50 insert on the donor plasmid and within mtd on the phage genome. All clones displayed adenine mutagenesis within the M50 sequence (FIG. 35), confirming that they resulted from DGR homing. Adenine-mutagenized homing products of the expected size resulting from complementation with pMX-M50 were also detected using primers P5/P2 (FIG. 36).

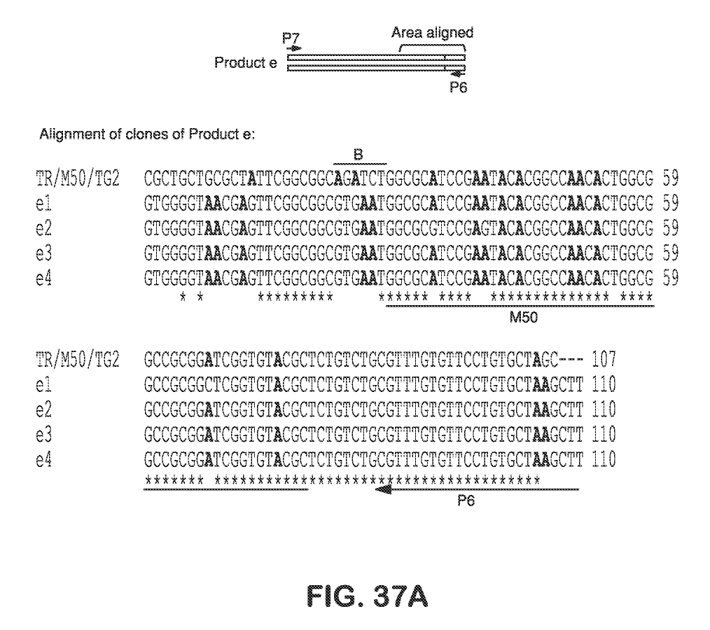

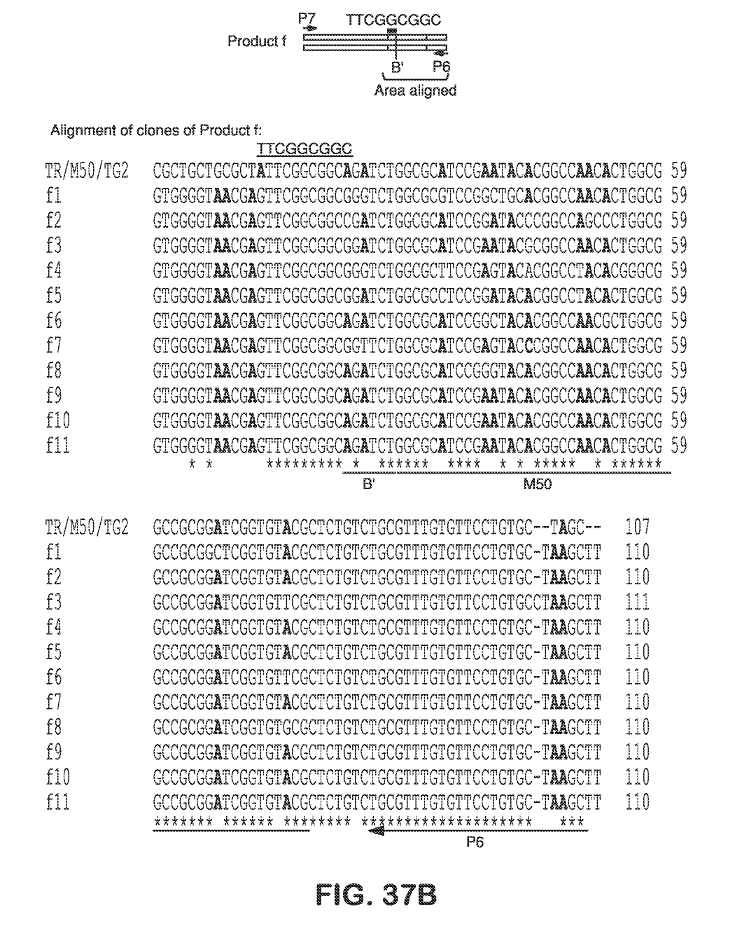

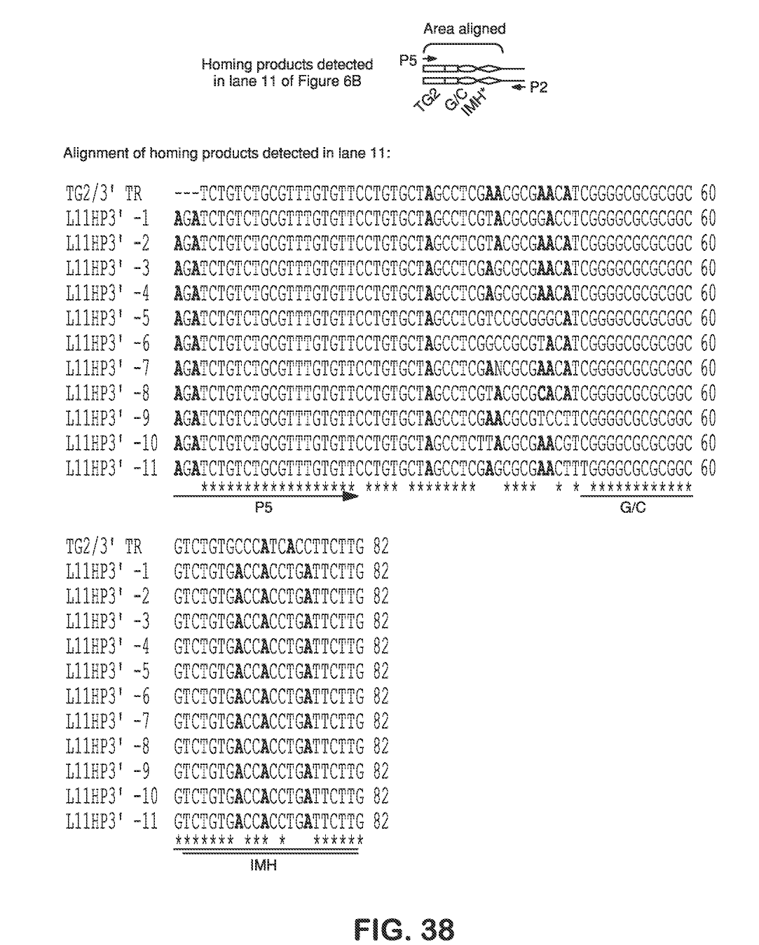

When tested in BPP-1dΔVR1-99 lysogens, pMX-ΔTR23-84 did not generate detectable homing products (FIG. 21B, lanes 4 & 10). In contrast to the experiment shown in FIG. 20, in which total DNA was used to identify homing intermediates, the assays in FIG. 21 used DNA isolated from intact phage particles to eliminate abortive products. Although pMX-ΔTR23-84 was capable of cDNA integration at the 3′ end of VR, the lack of integration at the 5′ end prevented packaging. In contrast, the presence of the 50 bp mtd segment in pMX-M50 efficiently restored Brt-dependent homing (FIG. 21B, lanes 5 & 6, 11 & 12). Sequence analysis of pMX-M50 homing products amplified with primers P7/P6 (FIG. 21B, lane 5, band 3) revealed two species of identical size. One corresponds to cDNA integration within the M50 region of mtd due to homology (FIG. 21C, e; FIG. 37A). The other species (FIG. 21C, f; FIG. 37B) resulted from cDNA integration at the same 9 nt sequence implicated in the generation of product d. Adenine mutagenesis was observed in the 50 bp mtd sequence in the majority of homing products derived from pMX-M50 (FIG. 37). Taken together, these results demonstrate that cDNA integration at the 5′ and of VR, upstream of the (G/C)₁₄ element, was homology-driven. Furthermore, only short stretches of nucleotide identity between the cDNA and target sequences were required to complete the homing reaction.

DGRs are a family of retroelements that use RT-mediated mobility to generate diversity in protein-encoding DNA sequences (Doulatov, S. et al., Nature, 431:476-481 (2004)). Our results demonstrate that DGRs have evolved an adaptation of TPRT which was site-specific, and capable of precisely regenerating target sequences. This “copy and replace” pathway allowed continuous rounds of protein diversification and the creation of new binding specificities for ligand-receptor interactions.

### The RNA Intermediate

We demonstrated that the BPP-1 TR can accommodate sequence insertions at multiple sites. Inserted sequences were not only transferred to VR, but they also underwent adenine mutagenesis. The observation that heterologous sequences can be diversified by a DGR has practical applications as discussed below. In the course of these experiments, we developed a sensitive and selective PCR assay that allowed the identification and characterization of VR sequences that have specifically undergone mutagenic homing.

By engineering a self-splicing group I intron into the BPP-1 DGR TR, we showed that homing occurs through a TR-containing RNA intermediate. This conclusion is based on the observation that precisely spliced, adenine-mutagenized exons were transferred to VR. Initially used in yeast to demonstrate retrotransposition of Ty1 (Boeke, J. D. et al., Cell, 40:491-500 (1985)), intron tagging has become a “gold standard” for identifying retrotransposition (Cousineau, B. et al., Cell, 94:451-462 (1998); Guo, H. et al., Science, 289:452-457 (2000); Moran, J. V. et al., Cell, 87:917-927 (1996)). Further experiments revealed sequence requirements for the RNA intermediate. Deletion analysis showed that internal sequences in TR are nonessential, with only ˜10 bp at the 5′ end and ˜38 bp at the 3′ end required for homing. The 10 bp at the 5′ end of TR formed part of an essential RNA structure and/or provided homology for cDNA integration into VR. The 3′-end requirements were composed of the (G/C)₁₄ and IMH* elements. Although sequences internal to TR were dispensable, regions of the RNA transcript important for homing extended upstream and downstream of TR. Synonymous mutations used here (FIG. 30), showed that sequences extending into the 3′ end of atd (˜42 bp upstream of TR), and the 5′ end of brt (˜194 bp downstream of TR) formed the boundaries of the RNA transcript required for maximum activity.

Mechanisms of cDNA Integration

We developed a marker coconversion assay, based on the introduction of single-nucleotide markers in TR, to genetically map cDNA integration sites at the 3′ and 5′ ends of VR. Sequence analysis of homing products revealed a narrow boundary for marker coconversion at the 3′ end, occurring within the (G/C)₁₄ element (FIG. 19C). This represented the sites at which cDNA synthesis initiated during homing. Priming occurred following DNA cleavage, generating a 3′-OH for TPRT. A TPRT mechanism for cDNA integration within the (G/C)₁₄ element explained the observation that mutagenesis only occurred upstream of this element; adenines in IMH* did not cause mutations in IMH, and IMH coconversion to IMH* was not observed (Doulatov, S. et al., Nature, 431:476-481 (2004); Liu, M. et al., Science, 295:2091-2094 (2002)). In contrast to the 3′ end, the boundary of marker coconversion at the 5′ end of VR was diffuse and indicated that cDNA integration occurred at virtually any position upstream of the point of initiation.

Our results demonstrated a sequential process in which cDNA integration initially occurred within the (G/C)₁₄ element, followed by 5′-end integration driven by TR/VR homology. To test this we generated a recipient phage lacking the first 99 bp of VR. This deletion included all VR sequences located upstream of the (G/C)₁₄ and IMH elements. PCR homing assays detected 3′-, but not 5′-end integration products, indicating that cDNA integration at the 3′ end of VR was independent of integration at the 5′ end. The ability to detect 3′-end integration was IMH-dependent, indicating that IMH dictated the unidirectional nature of sequence transfer by mediating 3′-end cDNA initiation. As no 5′-end integration products were identified, the 3′ integration products resulted from amplification of cDNA intermediates “trapped” in the homing reaction.

Complete homing into a BPP-1 derivative missing the first 99 bp of VR was restored by inserting a 50 bp homologous segment of mtd into TR (FIG. 21B). These data showed that cDNA integration at the 5′ end of VR was homology-driven and did not depend on specific VR sequences. By analyzing numerous products from this and other homing events, we discovered that short stretches of sequence homology as small as 4-12 bp were sufficient to mediate cDNA integration at sites upstream of the (G/C)₁₄ element.

As expected, transposition of the mtd segment from TR to VR was accompanied by efficient adenine mutagenesis of the heterologous sequences. RecA-mediated homologous recombination machinery was not required for DGR function, as a recA deletion had no effect on mutagenic homing or phage tropism switching. In this respect, DGR homing resembled group II intron homing in Escherichia coli and cDNA-mediated gene conversion by the Ty1 retroelement in Saccharomyces cerevisiae, which occur in the absence of RecA or its yeast homologs Rad51, Rad55 and Rad57, respectively (Cousineau, B. et al., Cell, 94:451-462 (1998); Derr, L. K., Genetics, 148:937-945 (1998)).

### A Model for DGR Function

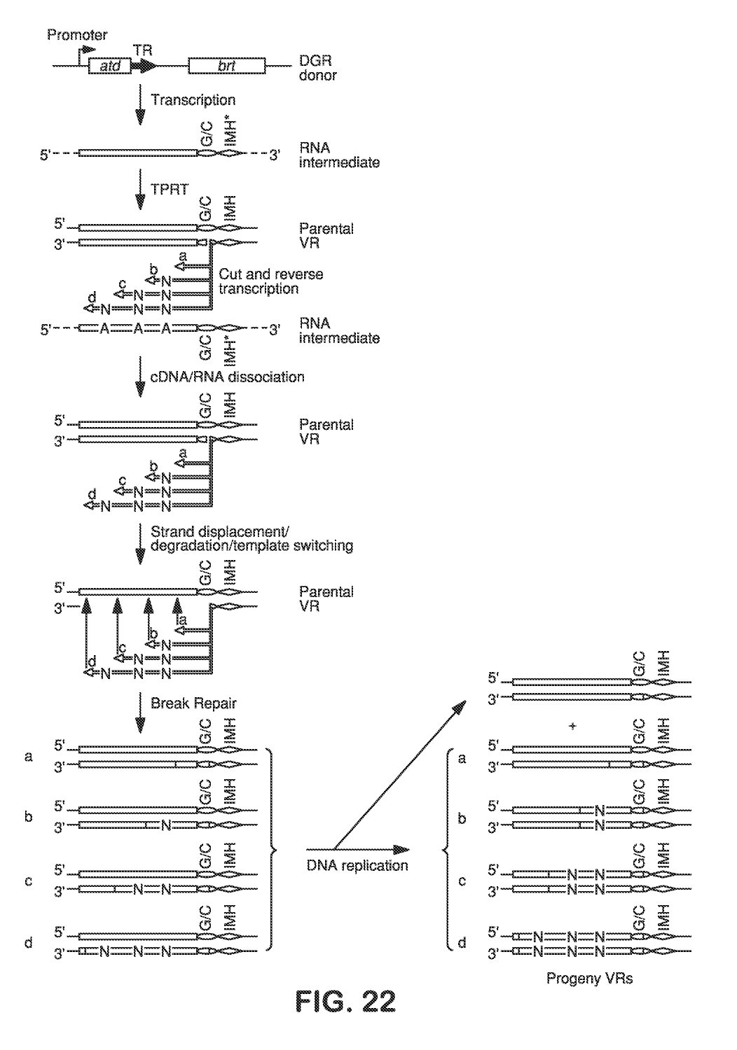

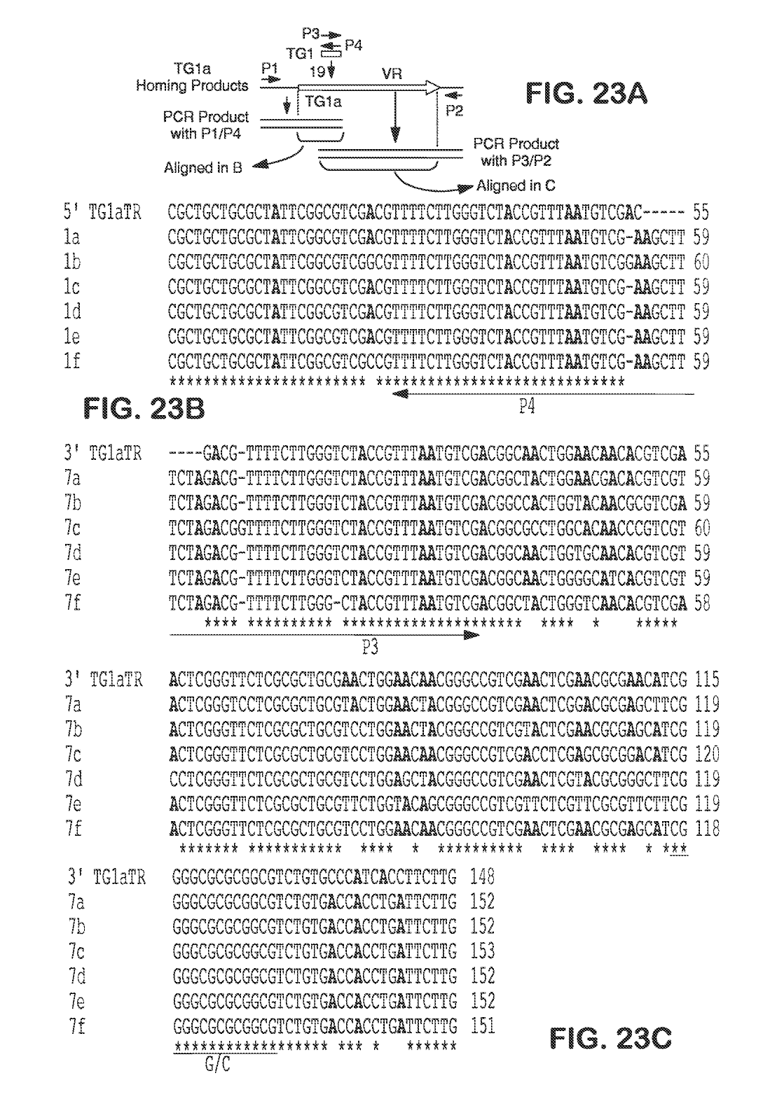

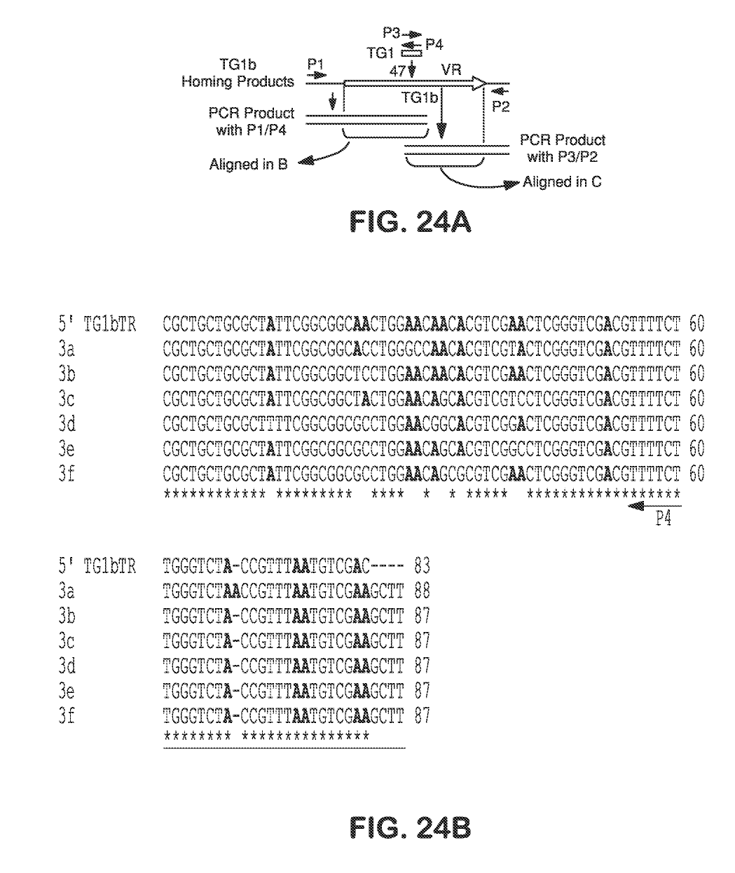

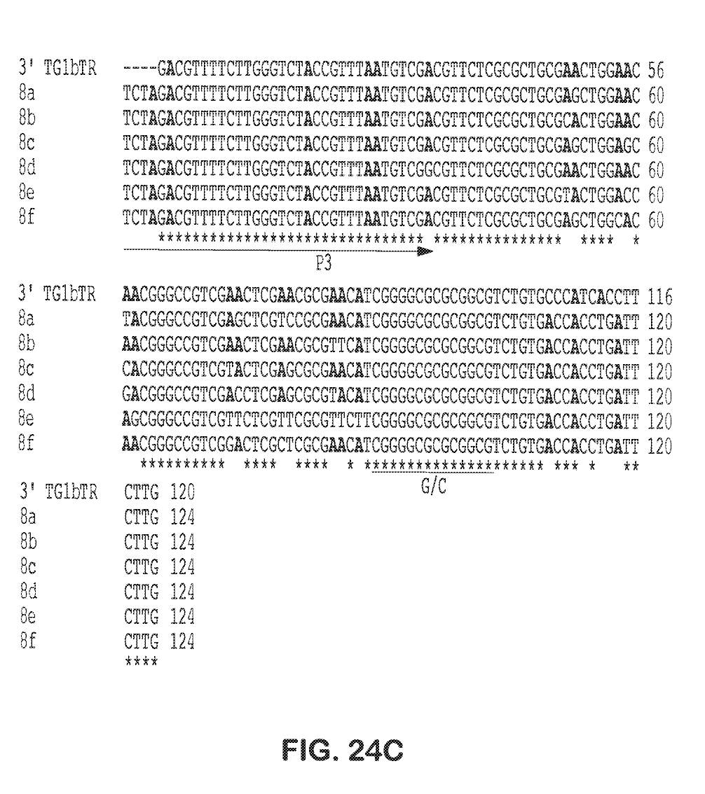

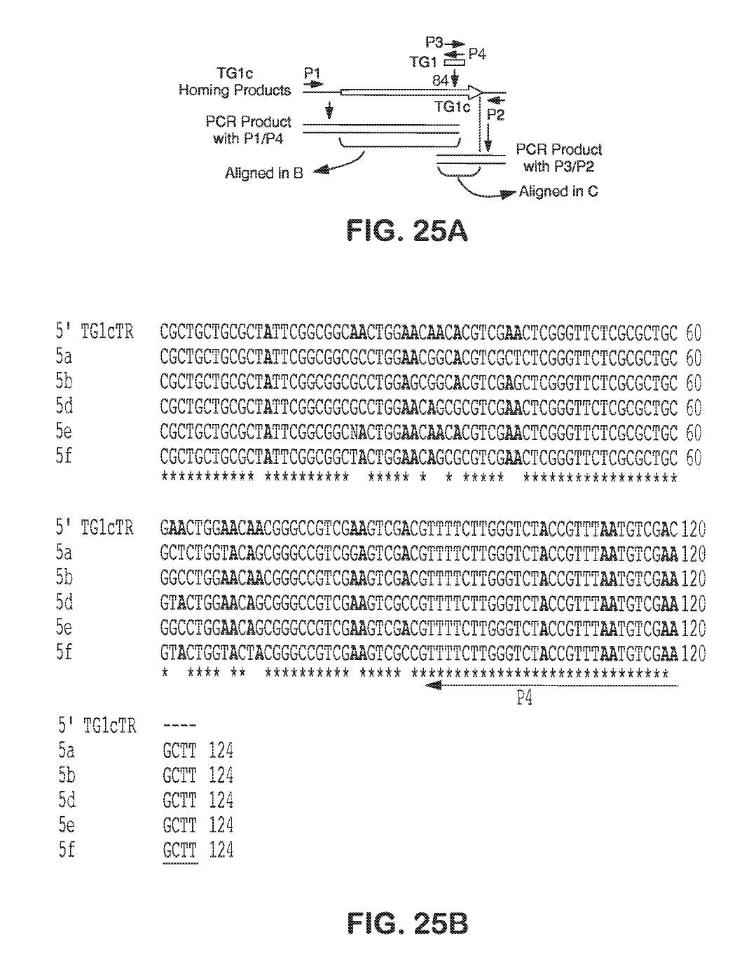

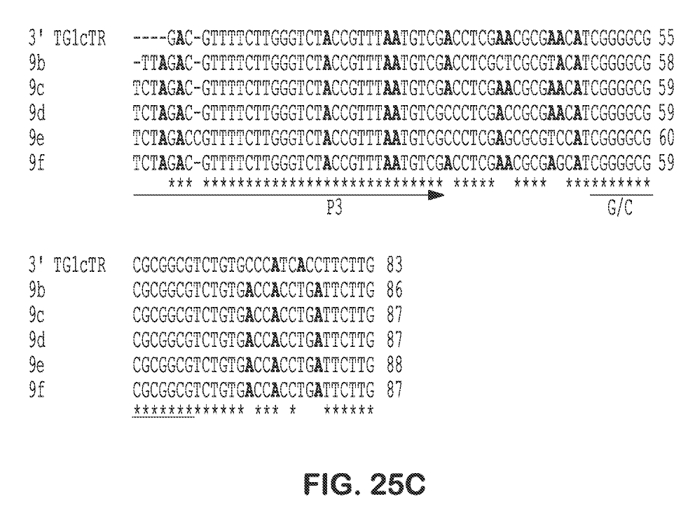

Our results support the DGR-mediated diversity generation outlined in FIG. 22. DGR homing was a site-specific retrotransposition process that did not lead to a copy number increase in either TR or VR. It initiated through a TPRT mechanism primed with either a single-stranded nick in the antisense strand, or a double-stranded break within the (G/C)₁₄ element. This led to a boundary of marker coconversion at the 3′ end of VR. Our data demonstrated that cDNA initiation involved sequence-specific recognition of the (G/C)₁₄ and IMH elements. It also demonstrated the existence of an endonuclease activity which provided by a protein and/or a catalytic RNA. Although cDNA initiation occurred within a boundary, the heterogeneity observed at position 109 (FIG. 19C) suggested a slight relaxation in the specificity of the proposed endonuclease. The model in FIG. 22 is consistent with other data, and with the close evolutionary relationships between DGRs and group II introns which are known to use TPRT for retrohoming (Doulatov, S. et al., Nature, 431:476-481 (2004); Lambowitz, A. M. and Zimmerly, S., Annu. Rev. Genet., 38:1-35 (2004)).

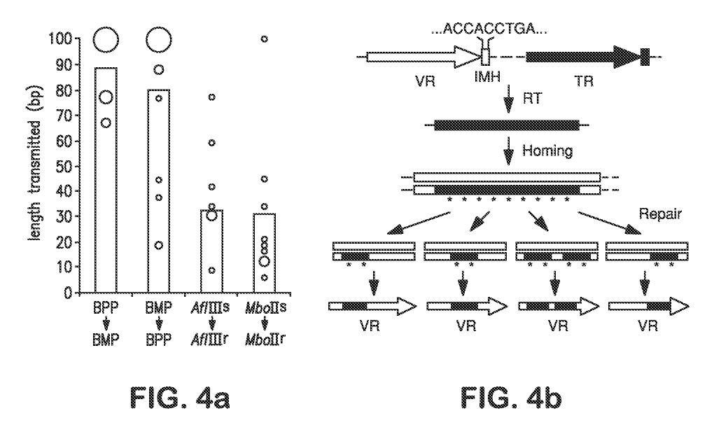

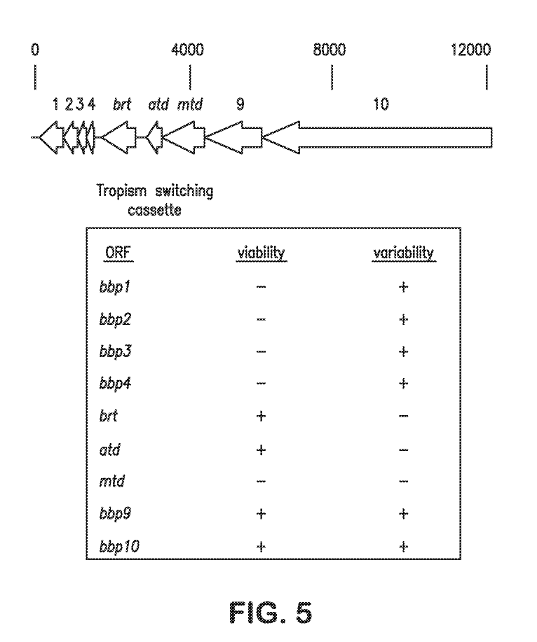

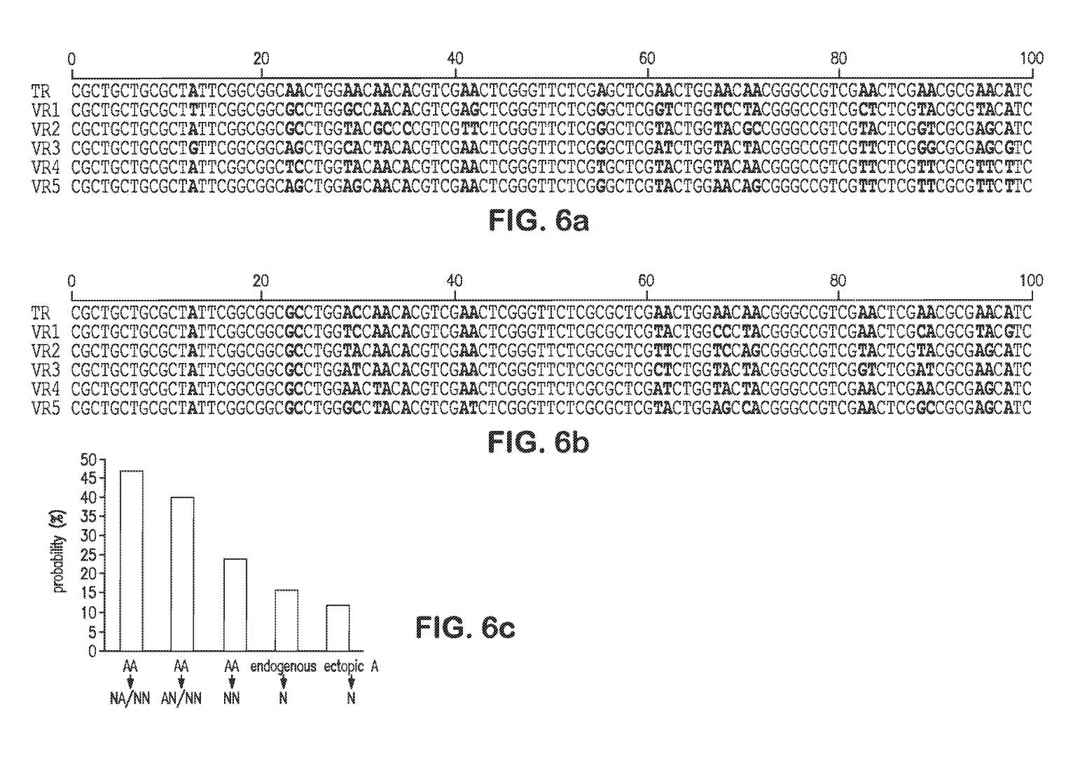

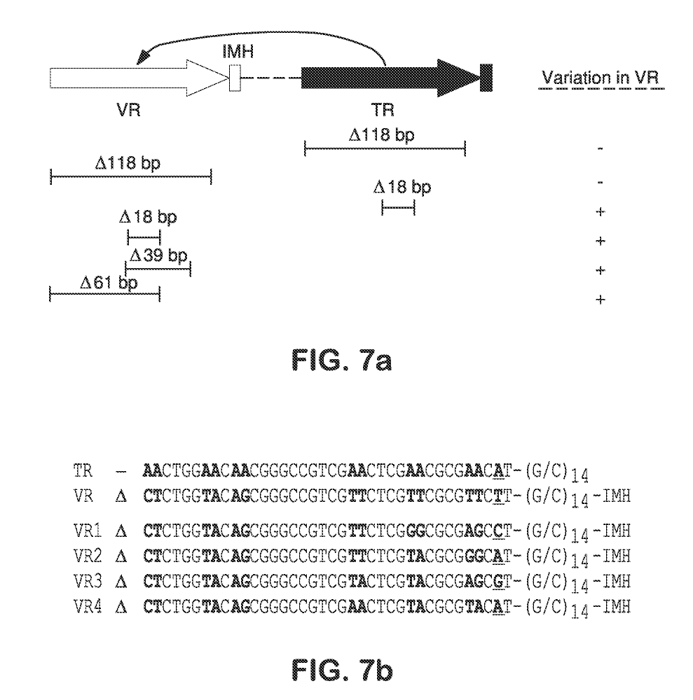

cDNA integration into VR sequences upstream of the (G/C)₁₄ element was homology-dependent and occurred through template switching or strand displacement (FIG. 22). The observation that 5′-end integration was mediated by very short stretches of sequence identity indicated that template switching was the primary pathway for cDNA integration into the 5′ end of VR. Such a process would not require RecA, and cDNA integration into VR before extending to the end of the TR RNA accounted for the diffuse boundary of marker coconversion observed at the 5′ end of VR (FIG. 4C). Once integration had occurred, the nascent minus-strand cDNA as was predicted contained mismatches at specific positions due to adenine mutagenesis. These were resolved via DNA replication to separate the two strands. Atd encoded an RNA-binding protein with an essential role in the homing reaction (Guo, H. et al., Science, 289:452-457 (2000), unpublished data).

Our results revealed several key features of the mutagenesis process. Sequence analysis of RT-PCR products derived from precisely spliced RNA transcripts of pMX-td, a functional donor plasmid containing a group I intron inserted in TR, demonstrated the presence of intact adenines in the RNA intermediate required for mutagenic homing. This, and the lack of identifiable sequences in DGRs that could potentially encode RNA modifying enzymes, argued against RNA editing as the basis for adenine mutagenesis. In contrast, analysis of cDNA intermediates shown in FIG. 20A provided clear evidence of nucleotide substitutions at positions corresponding to adenines in TR (FIG. 33). This indicated that adenine mutagenesis occurs early in homing, during minus-strand cDNA synthesis initiated at the 3′ end of VR, before integration at the 5′ end. Our data also allowed us to measure the efficiency of adenine mutagenesis relative to homing. When sequence tags in TR were used to amplify VR sequences that have undergone homing, no selection was imposed for adenine mutagenesis. Nonetheless, over 90% of the products collectively analyzed contained substitutions at positions corresponding to adenines in TR. This indicated that adenine mutagenesis accompanied most homing events, and that the limiting step in generating diversity was the initiation or completion of the homing reaction. Taken together, our results were consistent with the hypothesis that adenine mutagenesis was an inherent property of the BPP-1 encoded Brt protein, and we predicted the same is true for RTs encoded by other DGRs.

Our models for TPRT at the 3′ end of VR, and short homology-mediated cDNA integration at the 5′ end, bore similarities to the site-specific retrotransposition mechanism of the R2 element of Bombyx moil (R2Bm) (Eickbush, D. G. et al., Mol. Cell. Biol., 20:213-223; Eickbush, T. H., Origin and Evolutionary Relationships of Retroelements, In The Evolutionary Biology of Viruses, S. S. Morse, ed. (New York: Raven Press, Ltd.), pp. 121-157 (1994)). A seminal difference, however, was that retrotransposition of R2Bm and similar elements led to destruction of their target sites. In contrast, DGR activity precisely regenerated sequences essential for both 3′- and 5′-end cDNA integration, thus preserved the ability to undergo repeated cycles of mutagenic homing and protein diversification. To date, over 40 DGRs have been identified in bacterial, phage and plasmid genomes, and it appeared that nature has adapted these elements to perform a diverse array of functions (Medhekar, B. and Miller, J. F., Curr. Opin. Microbiol., 10:388-395 (2007)). We have demonstrated that the Bordetella phage DGR was flexible and tolerated sequence insertions in TR. Most importantly, transposition of heterologous sequences was accompanied by efficient adenine mutagenesis. Furthermore, cDNA initiation at the 3′ end of VR was sequence-specific and had the (G/C)₁₄ and IMH elements, while cDNA integration upstream of these elements was homology-driven, but sequence independent. These properties indicated that DGR-mediated targeted evolution could be directed to desirable heterologous sequences by placing them upstream of the (G/C)₁₄ and IMH elements and by appropriately constructing hybrid TRs. As DGRs were continually capable of generating vast amounts of diversity, they provided significant advantages over synthetic library-based approaches for generating diverse protein repertoires and new protein functions.

Having now generally described the invention, the same will be more readily understood through reference to the following examples which are provided by way of illustration, and are not intended to be limiting of the present invention, unless specified.

### EXAMPLES

### Example 1

### Materials and Methods

Bacterial strains, phage and plasmids.

B. bronchiseptica strains were derived from the sequenced RB50 strain (Uhl et al. and Parkhill, J. et al. Comparative analysis of the genome sequences of Bordetella pertussis, Bordetella parapertussis and Bordetella bronchiseptica. Nat Genet. 35, 32-40 (2003)) and BPP-1 was induced from a rabbit isolate of B. bronchiseptica (Liu et al. 2002). BMP-1 was isolated from BPP-1 using the tropism switch assay (see below). Plate lysates were prepared using the soft-agar overlay method (Adams, M. H. Bacteriophages. (Interscience Publishers Inc, New York, N.Y., 1959) and tropism switch assays were performed as described previously (Liu et al. 2002). Bacterial and phage constructs were generated using allelic exchange (Edwards, R. A., Keller, L. H., Schifferli, D. M. Improved allelic exchange vectors and their use to analyze 987P fimbria gene expression. Gene 207, 149-157 (1998) and Figurski, D. H. & Helinski, D. R. Replication of an origin-containing derivative of plasmid RK2 dependent on a plasmid function provided in trans. Proc Natl Acad Sci USA 76, 1648-1652 (1979).

Multiple Substitution Constructs.

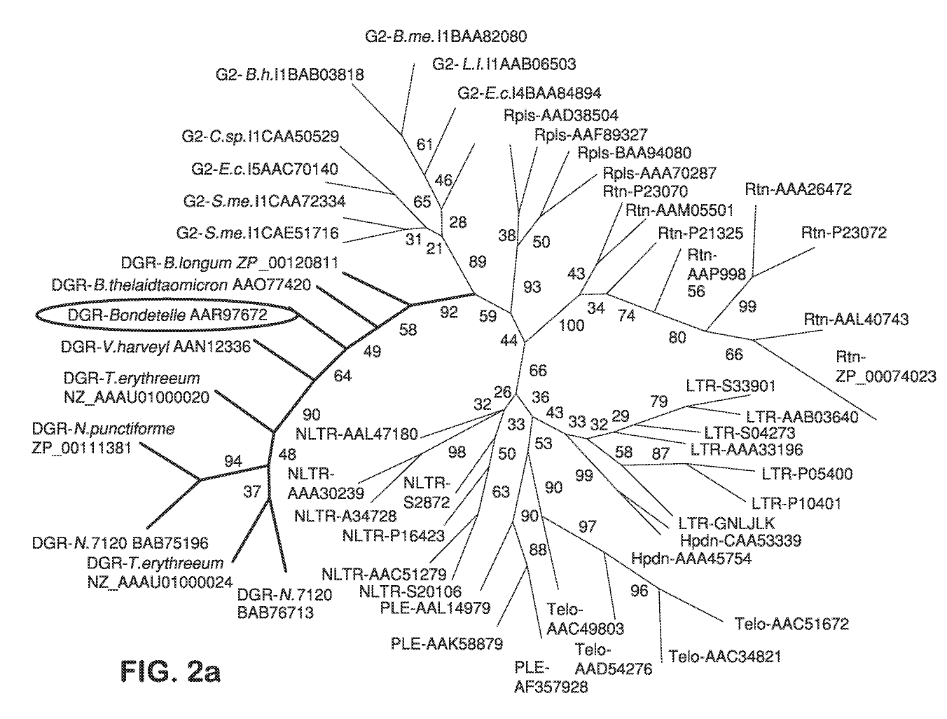

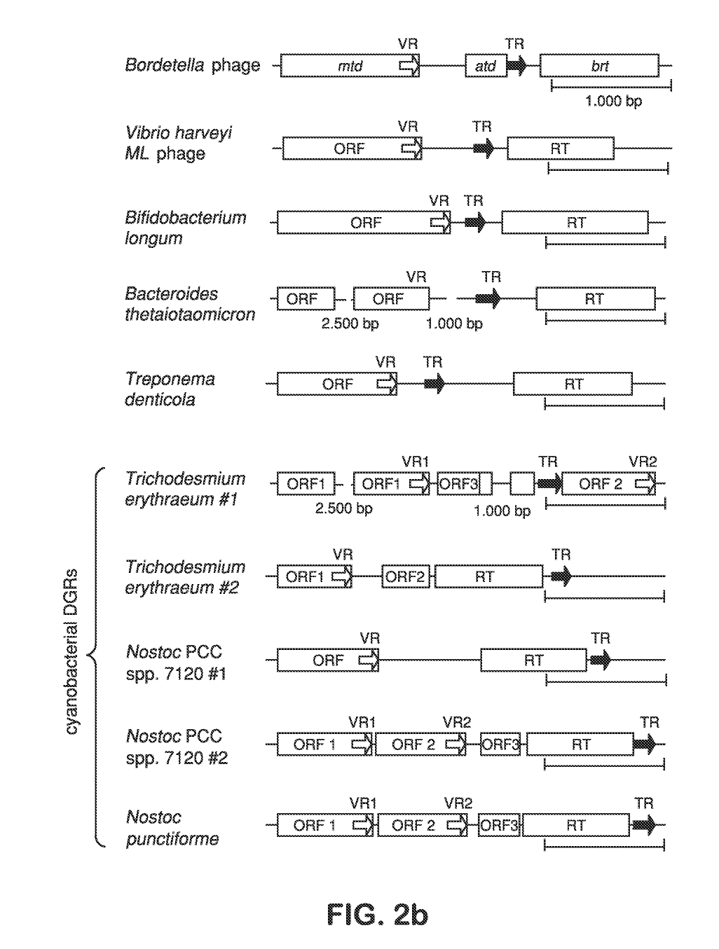

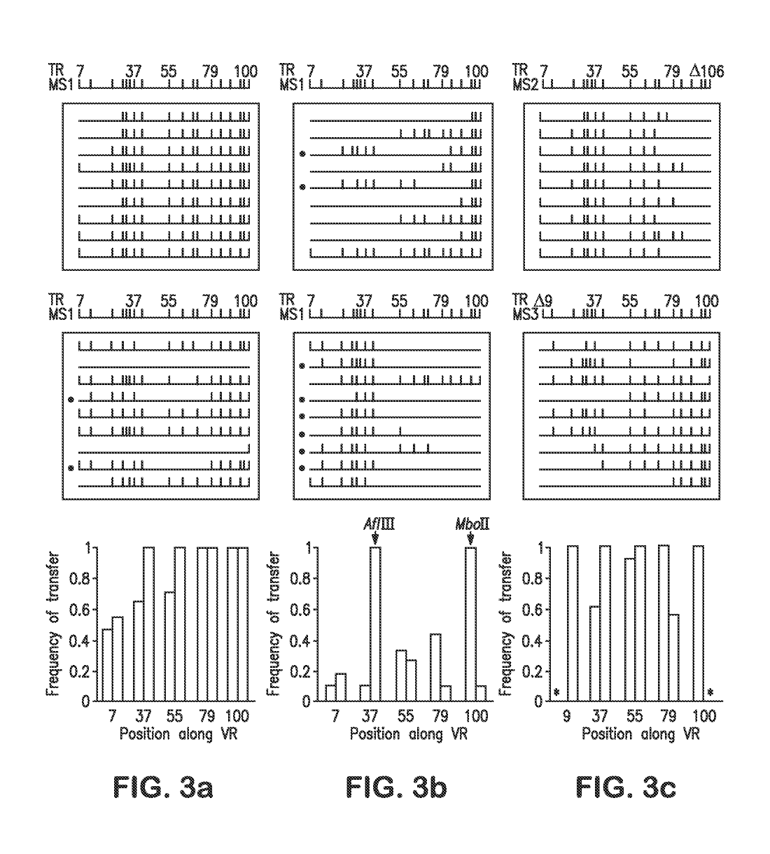

BPP-MS1 and BMP-MS1 (FIGS. 3a and 3b) are BPP-1 and BMP-1 derivatives, respectively, containing the following synonymous substitutions in TR: T7-A (PstI), G37-T (BstXI), C55-A (XhoI), C79-G (Apal) and G100-C (NlaIII). Each substitution generates a restriction site as indicated. The substitutions at positions 37 and 100 eliminate AflIII and MboII restriction sites, respectively, allowing in vitro selections for variability (FIG. 3b). Phage MS2 (FIG. 3c) is a Bpp-1 derivative containing a 1 bp deletion at position 106 in TR and substitutions: T7-A, G37-T, C55-A, C-79G. Phage MS3 (FIG. 2c) is a BMP-1 derivative containing a 1 bp deletion at position 9 in TR and substitutions: G37-T, C-55A, C79-G and G100—C.

In Vitro Variability Assays.

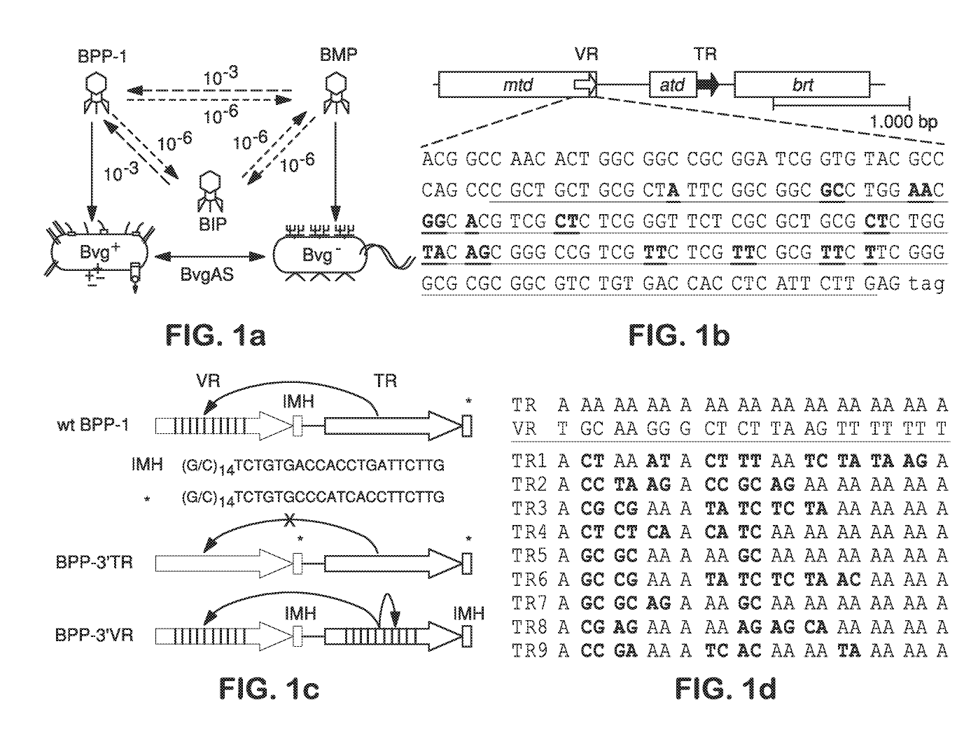

In vitro variability assays select for transfer, from TR to VR, of single nucleotide substitutions that confer resistance to restriction enzyme cleavage. Lysogens were induced with mitomycin C and VR sequences were amplified by PCR and digested with the appropriate restriction enzymes. The amplification-restriction cycle was repeated with nested primers until no further cutting was observed and the products were cloned into pBluescript KS+ vector (Stratagene) for sequencing. Variability in TR due to “self-homing” (FIG. 1c, BPP-3′VR) was assayed using BsrI, which cleaves the parental TR but not TR sequences with adenine modifications that confer resistance. For the multiple substitution experiments in FIG. 3b, amplification products were purified and digested with AflIII for BMP-MS1 phage or MboII for BPP-MS1 phage. In both cases, parental VR sequences are subject to cleavage whereas phage in which specific synonymous substitutions that eliminate restriction enzyme cleavage sites are transferred from TR are resistant.

Bioinformatics.

Annotated BPP-1 sequence is available under GenBank accession number AY029185. Database entries containing the conserved reverse transcriptase catalytic domain (Pfam 00078 rvt) were compiled and phylogenetic profiles were constructed using PHYLIP software package (at evolution.genetics.washington.edu/phylip.html). Entries that grouped together with Brt were searched for the presence of direct repeats proximal to the RT using REPuter program (Kurtz, S. et al. REPuter: the manifold applications of repeat analysis on a genomic scale Nucleic Acids Res. 29, 4633-2642 (2001)). Artemis software was used to collect data and facilitate annotation (Rutherford, K. et al. Artemis: sequence visualization and annotation. Bioinformatics 16:944-945 (2000)).

### Example 2

### Multiple Synonymous Substitutions

A genetic strategy for tracking events that give rise to sequence variants was designed based on the observation that conservative nucleotide substitutions in TR are incorporated into VRs of phages that have switched tropism. By introducing multiple synonymous substitutions positioned along TR, the portion of TR transferred during a switching event can be determined by recording the pattern of substitutions appearing in VR. Mechanistic events that underlie tropism switching can then be reconstructed from the resulting “haplotype” profiles.

Information was observed as not being transmitted evenly across VR. FIG. 3a shows the patterns of transmission accompanying BPP-->BMP/BIP or BMP-->BPP tropism switching. In both cases, 3′ markers were transmitted with 100% efficiency whereas 5′ markers were transmitted at frequencies approaching 50%. Variability at adenines correlated with the transfer of proximal substitutions, while lack of variability correlated with their absence. In several cases, mosaic patterns were observed in which stretches of variable, TR-derived sequence were interrupted by non-variant, VR-derived sequence (bullets, FIG. 3a). Together, these results argue against a simple cut and paste mechanism as commonly observed in transposition reactions (Pena, C. E., Kahlenberg, J. M., Hatfull, G. E. Assembly and activation of site-specific recombination complexes. PNAS 97, 7760-5 (2000) and Hallett B., Sherratt, D. J. Transposition and site-specific integration: adapting DNA cut- and paste mechanisms to a variety of genetic rearrangements. FEMS Microbiol Rev 21, 157-78).

Because the sequence determinants that govern receptor specificity are unclear, tropism switching assays are inherently biased by a powerful, yet poorly defined, set of selective pressures. Substitution patterns were therefore recorded using PCR-based in vitro assays that select for variability at single, precisely defined positions, with no selection for tropism switching or phage infectivity. These assays are based on the loss of restriction sites in parental VR sequences that result from the transmission of synonymous substitutions in TR.

As shown in FIG. 3b, in vitro variability assays revealed selection-specific patterns of marker transfer in which AflIII-selected clones preferentially transferred the middle portion of TR containing the selected site (position 37), and transfer frequencies precipitously fell in either direction. MboII-selected clones transferred the 3′ end of TR which contains the selected site (position 100), but were indifferent to sequence variation at the 5′ end. In both cases, maximal frequencies of marker transfer were shifted to the exact point of selection. The majority of events displayed either interrupted patterns of transmission or patches of transmission flanked by invariant sequence (bullets, FIG. 3b).

Despite the lack of selection for mutagenesis, all of the VR sequences in FIG. 3b contain adenine-substitutions. To further probe the extent of plasticity, a strong negative selection against transfer of the 3′ or 5′ boundaries of TR was imposed. This was accomplished by the introduction of frameshift mutations which, if transferred, produce non-viable phage.

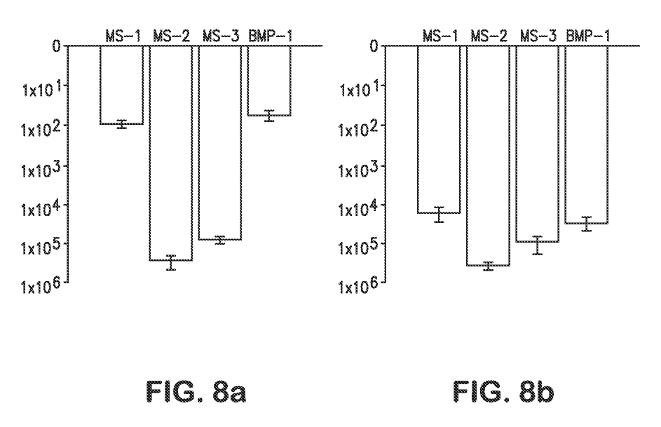

The system surprisingly accommodated these rather extreme selections. Both mutant phages were able to switch tropism while avoiding the transmission of frameshift mutations, generating transmission histograms that are essentially mirror images (FIG. 3c and FIG. 8).

### Example 3

### Gene Conversion

Selection at a single position, as imposed by in vitro restriction enzyme-based assays, tends to isolate shorter variable sequences centered around the point of selection. More complex selections for novel receptor specificity select for larger segments of transferred, mutagenized sequence (FIG. 4a).

These conditions could be satisfied by a mechanism in which site-specific homing, initiated at IMH, is followed by random gene conversion due to recombination or repair. According to this model, a heteroduplex is formed at VR during the variability generating process (FIG. 4b). See Morrish, et al. and Wank, et al. The heteroduplex would be characterized by a high density of mismatched basepairs resulting from the hybridization of VR with a TR-derived cDNA. Mismatch repair, or an analogous process, would give rise to chimeric VRs containing “patches” of sequence variation.

A consequence of the diversity-generating mechanism is that variability is introduced into the mtd locus in a highly targeted manner. Diversification exclusively occurs within the boundaries of the variable repeat, it only occurs at positions corresponding to adenine residues in TR, and it can be limited to the subset of bases that are subject to selection. This “focusing” of variability has the potential to be highly adaptive as it provides a means to efficiently respond to selective pressures while minimizing the accumulation of unnecessary or deleterious substitutions. The repair step may be essential given the high rate of adenine-mutagenesis, and it allows optimization of receptor specificity through iterative rounds of selection (Wrighton, N. C. et al. Small peptides as potent mimetics of the protein hormone erythropoietin. Science 273, 458-64 (1996) and Fairbrother, W. J. et al. Novel peptides selected to bind vascular endothelial growth factor target the receptor-binding site. Biochemistry 37, 17754-17764 (1998)).

### Example 4

### Related Gene Diversification Systems in Other Organisms

The ability to diversify protein domains involved in ligand-receptor interactions has extremely broad utility. The invention thus provides elements homologous to the Bordetella phage retroelement as discovered from other sources in nature. To identify related sequences, open reading frames (ORFs) of bacterial origin containing conserved RT domains were compiled. A subset clustered phylogenetically with Brt (FIG. 2a). Adjacent sequences were examined and in all cases candidate TR and VR repeats were identified, with VRs located at the 3′ end of an ORF.

Further annotation revealed an array of cassettes which we now designate as putative diversity generating retroelements (DGRs). Although RT domains are highly related, and DGRs share an overall conservation of structural features (FIG. 4b), there is little if any sequence similarity between other components of these related cassettes.

In every case VR analogs differ from their cognate TRs almost exclusively at positions corresponding to adenines (FIG. 9). This observation supports the use of these cassettes based on their function to generate diversity in a similar manner.

Comparison of the 3′ ends of cognate VRs and TRs also suggests the presence of analogous sequences to the Bordetella phage IMH site (FIG. 9). As shown in FIG. 2b, DGRs are found in the chromosomes of a wide array of bacterial species and they display variations on a common theme. For example, Nostoc and Trichodesmium species contain cassettes in which a single TR apparently supplies two different VRs with sequence variability. In such cases, the VRs are part of paralogous ORFs with over 90% sequence identity and are identical except for bases corresponding to adenines in TR.

In addition, several cyanobacterial species contain multiple DGRs which are not homologous and have, therefore, been independently acquired. Although the Bordetella and V. harveyi cassettes are present on prophage genomes, there is no evidence of phage association for the remaining sequences. On the basis of the data in FIG. 2, it is proposed that DGRs have evolved to perform myriad functions in diverse organisms.

Retroelements such as group II introns (Bonen, L. & Vogel, J. The ins and outs of group II introns. Trends Genet. 17, 322-331 (2001)), retrotransposons (Bushman, F. D. Targeting survival: integration site selection by retroviruses and LTR retrotransposons Cell 115, 135-138 (2003)), retroviruses (Gifford, R. & Tristem, M. The evolution, distribution and diversity of endogenous retroviruses. Virus Genes 26, 291-315 (2003)), and human LINEs (Kazazian, H. H. Jr. & Goodier, J. L. LINE drive: retrotransposition and genome instability Cell 110, 277-280 (2002)) share related characteristics.

### Example 5

### Elements of the DGR which Act in cis and trans

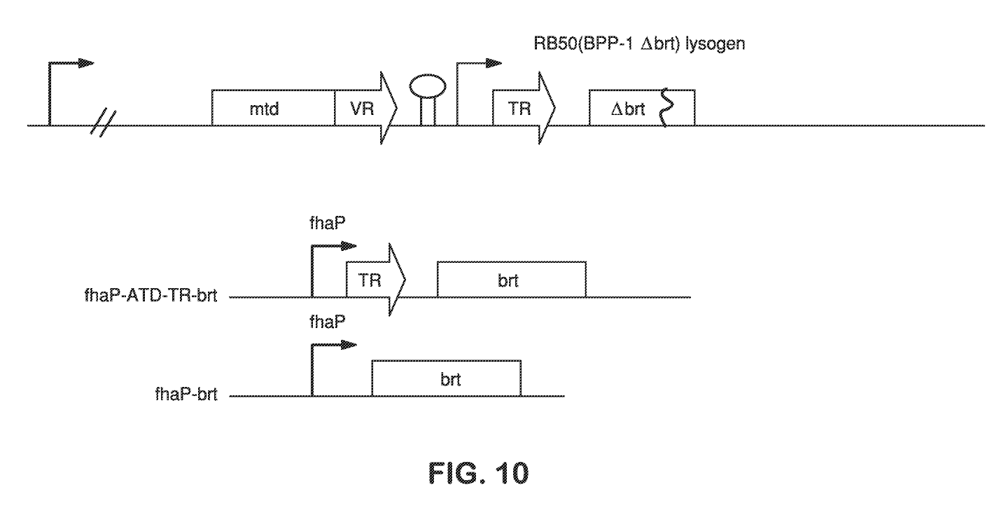

Bordetella strain 61-11(RB50 BPP-1 Δbrt, see FIG. 10) was used to characterize cis and trans acting elements of the DGR. The strain carries a deletion in the prophage RT gene (brt) which renders the phage unable to switch its tropism.

DNA fragments containing various components of the DGR were amplified by PCR from the intact RB50 BPP-1 lysogen, digested with restriction enzymes and cloned into the vector pBBRmcsF carrying an fha promoter. Two of the plasmids, pfhaP-atd-TR-brt and pfhaP-TR-brt, are shown in Table 1 and schematically in FIG. 10. In pfhaP-atd-TR-brt, there is no terminator between the fhaP and atd-TR-brt sequences. In pfihaP-TR-brt, there is no atd between the tha promoter and the TR sequence.

The resulting constructs are listed in Table 2.

TABLE 2

| | | Primers used for | Causes tropism switching |
|---|---|---|---|
| plasmid: | | constructions: | in strain 61-11: |
| pfhaP-atd-TR- | the atd-TR-brt region in DGR is | atd-TRHindIII for | yes |
| brt | cloned in vector pBBRmcsF | BrtBamHrev | |
| pfhaP-TR-brt | the TR-brt region in DGR is cloned in | TRHindIII for | yes |
| | vector pBBRmcsF | BrtBamHrev | |
| pfhaP-brt | only the brt region in DGR is cloned | BrtXbaI for | no |
| | in vector pBBRmcsF | BrtSacI rev | |
| pfhaP-atd-TR | the atd-TR region in DGR is cloned in | | no |
| | vector pBBRmcsF | | |

After transformation of plasmids into strain 61-11, tropism switching was assayed by inducing lysogenic cells with mitomycin C and plating the phage lysate directly onto RB53 (a Bvg+ strain) or RB54 (a Bvg− strain) to observe plaque formation (see FIG. 11 for a representation of the use with pfhaP-atd-TR-brt).

Induced lysate from cells harboring the pfhaP-atd-TR-brt was plated directly on RB53(Bvg+) or RB54(Bvg−). Eighteen (18) plaques from RB53 plates were isolated and, after PCR amplification, their VR regions were sequenced and found to have changes at positions corresponding to adenines in the TR. From 100 μl of lysate, an average of 15 plaques were seen by plating directly on RB54 (efficiency compared to plating on RB53 is about 10⁻³).

In parallel, induced lysate from cells harboring the pfhaP-TR-brt was also directly plated on RB53(Bvg+) or RB54(Bvg−). Phages from 10 plaques from RB53 plates were isolated, their VR regions amplified by PCR and sequenced. All had changes in VR regions corresponding to adenines in the TR. From 100 μl of lysate, an average of 15 plaques were seen by plating directly on RB54 (efficiency compared to plating on RB53 is about 10⁻³).

Because the VR regions of all of the phages, even those that did not switch tropism, contained nucleotide changes corresponding to adenine residues in the TR, the frequency of mutagenesis was effectively 100% with the use of a strong heterologous promoter.

Similar experiments with pfhaP-brt and pfhaP-atd-TR showed no tropism switching.

The results show that the minimal unit for complementation of the brt deletion, restoring the ability to switch tropism, is the TR-brt region, in which:
(i) The TR acts in cis with brt
(ii) The TR acts in trans to the VR

The results further suggest that the trans acting construct was able to direct the mutagenesis of a proviral copy of the phage VR sequence.

### Example 6

### Mutagenesis in trans of an Uninduced Prophage

The ability of trans expression of TR-brt to alter the VR sequence of a (chromosomal) prophage was determined in the absence of phage induction. In the uninduced lysogen 61-11 harboring the plasmid pfhaP-atd-TR-brt, PCR amplification was performed on an overnight culture and DNA products were cloned into a sequencing vector.

In one experiment, one colony was picked and grown by overnight culture in LB medium at 37° C. The VR region of 511 overnight culture was PCR amplified and cloned into a sequencing vector (pBluesriptII). 20 plasmids were sequenced, with 2 (thus 10%) having changes in the VR corresponding to adenines in the TR. In another experiment, 5 colonies were picked and individually grown via overnight culture in LB medium at 37° C. The VR region of 511 of each overnight culture was PCR amplified and cloned into a sequencing vector (pBluesriptII). Three (3) plasmids from each plating were sequenced, with 5 of 15 (thus 30%) having changes in the VR corresponding to adenines in the TR.

Thus, fha promoter-directed transcription of the TR-brt region results in elevated levels of VR mutagenesis, demonstrating that:
(i) TR-brt transcription can be placed under the control of a heterologous promoter, replacing the need for the aid element (see below)
(ii) Control of TR-brt transcription affects the levels of VR mutagenesis
(iii) The TR-brt region can act in trans on a cognate VR in the bacterial chromosome.

### Example 7

### Introduction of Added Sites of Mutagenesis

Using site-directed mutagenesis, 3 adenines were substituted for nucleotides 59-61 of the TR region. The corresponding VR nucleotides encoded non-variable Mtd residue A356. Using homologous recombination, the TR with 3 adenines substituted was introduced into strain 6405 (RB54 BMP-1 lysogen). Successful modification of the 6405 TR was confirmed by sequencing and restriction digestion, generating strain 6405AAA (see below).

TR-strain 6405
cgctgctgcgctattcggcggcaactggaacaacacgtcgaactcgggtt
ctcgcgctGCGaactggaacaacgggccgtcgaactcgaacgcgaacatc
ggggcgcgcggcgtctgtgcccatcaccttcttg
TR-strain 6405AAA
cgctgctgcgctattcggcggcaactggaacaacacgtcgaactcgggtt
ctcgcgctAAAaactggaacaacgggccgtcgaactcgaacgcgaacatc
ggggcgcgcggcgtctgtgcccatcaccttcttg

Strain 6405AAA was induced, VR regions of the resulting phage mixture were PCR amplified and digested with a restriction enzyme (MboII) that cuts the parental VR sequence 3′ to the AAA substitution. The in vitro selection was for diversification of the parental MboII recognition sequence without assessing its effect on the encoded polypeptide. Re-amplification of VR sequences undigested by MboII followed by cloning and sequencing demonstrated that the newly introduced TR adenine residues were transmitted to VR and diversified.

### Example 8

### The atd is not Required for Homing Mutagenesis

Placement of a stop codon into atd does not eliminate mutagenesis. This indicates that the atd does not encode a protein for mutagenesis.

Using site-directed mutagenesis, a stop codon was substituted for the 9th amino acid of the postulated accessory tropism determinant (atd) ORF. Using homologous recombination, the atd with a stop codon was introduced into lysogen strain 6405. Successful modification of the 6405 was confirmed by sequencing.

After induction and an additional round of propagation, phages able to plaque on either BVG+ and BVG− Bordetella bronchiseptica were isolated. Therefore, the phage maintained the ability to switch tropism. In addition, the primary induction of phage produced variants. This was shown by selecting for variants in the primary lysate using altered sensitivity to restriction digest in a restriction enzyme/PCR selection method.

Combined with the results of Example 5 above, one can conclude that an atd encoded polypeptide is not required for tropism switching and the atd sequence can be entirely substituted by a heterologous promoter.

atd-Wild type
atggaacccatcgaggaagcgacaAAGtgctacgaccaaatgctcattgt
ggaacggtacgaaagggttatttcgtacctgtatcccattgcgcaaagca
tcccgaggaagcacggcgttgcgcgggaaatgttcctgaagtgcctgctc
gggcaggtcgaattattcatcgtggcgggcaagtccaatcaggtgagcaa
gctgtacgcagcggacgccgggcttgccatgctgcgattttggttgcgct
ttctcgcgggcattcagaaaccgcacgctatgacgccgcatcaggtcgag
acagcacaagtgctcatcgccgaagtggggcgcattctcggctcctggat
tgcccgcgtgaatcgcaaagggcaggctgggaaataa
atd-with stop codon
atggaacccatcgaggaagcgacaTAGtgctacgaccaaatgctcattgt
ggaacggtacgaaagggttatttcgtacctgtatcccattgcgcaaagca
tcccgaggaagcacggcgttgcgcgggaaatgttcctgaagtgcctgctc
gggcaggtcgaattattcatcgtggcgggcaagtccaatcaggtgagcaa
gctgtacgcagcggacgccgggcttgccatgctgcgattttggttgcgct
ttctcgcgggcattcagaaaccgcacgctatgacgccgcatcaggtcgag
acagcacaagtgctcatcgccgaagtggggcgcattctcggctcctggat
tgcccgcgtgaatcgcaaagggcaggctgggaaataa

### Example 9

### Diversification of a Heterologous Polypeptide

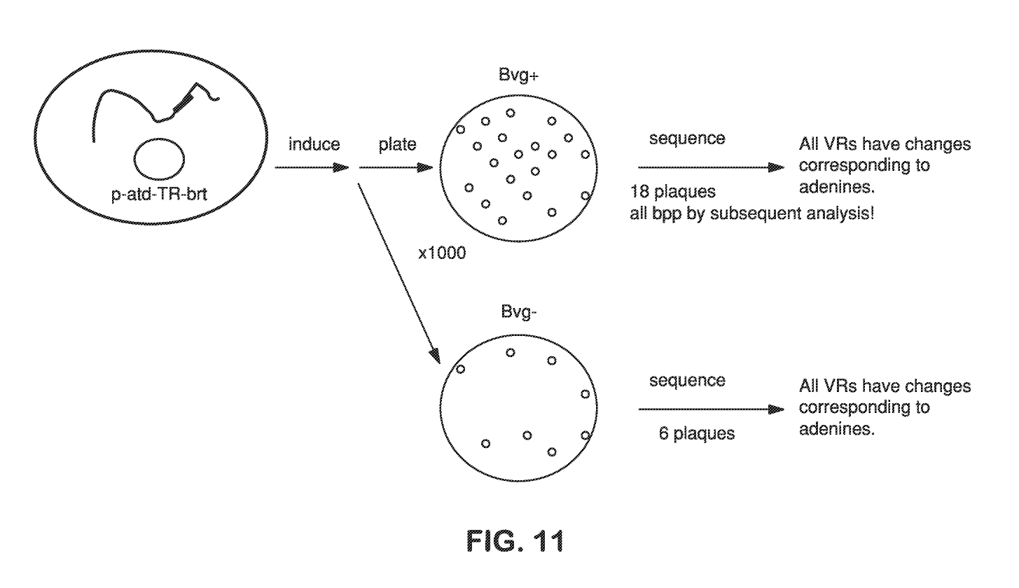

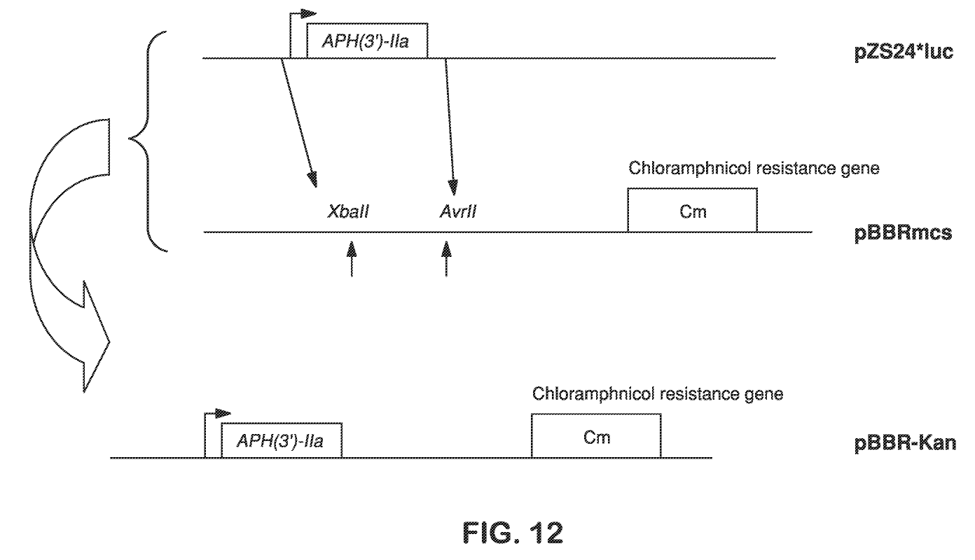

A kanamycin resistance gene encoding aminoglycoside-3′-phosphotransferase-II (APH(3′)-IIa) with its own promoter was isolated from plasmid pZS24*luc using restriction enzymes SacI and XbaI and cloned into plasmid pBBRmcs (FIG. 12). The E. coli strain XL1-blue carrying this new plasmid pBBR-Kan was able to grow in presence of both kanamycin and chloramphenicol.

The amino acid sequence of APH(3′)—IIa is 264 residues long and is as follows: M I E Q D G L H A G S P A A W V E R L F G Y D W A Q Q T I G C S D A A V F R L S A Q G R P V L F V K T D L S G A L N E L Q D E A A R L S W L A T T G V P C A A V L D V V T E A G R D W L L L G E V P G Q D L L S S H L A P A E K V S I M A D A M R R L H TL D P A T C P F D H Q A K H R I E R A R T R M E A G L V D Q D D L D E E H Q G L A P A E L F A R L K A R M P D G E D L V V T H G D A C L P N I M V E N G R F S G F I D C G R L G V A D R Y Q D I A L A T R D I A E E L G G E W A D R F L V L Y G I A A P D S Q R I A F Y R L L D E F F.

The Leu residue at position 243 is shown with emphasis.

A stop codon (taa) was introduced into position 243 by using site-directed mutagenesis. The mutation eliminated kanamycin resistance in a host harboring the plasmid pBBR-Kan. Plasmid pZS24*luc is from: Lutz, R. & Bujard, H. (1997) Nucleic Acids Res. 25, 1203-1210.

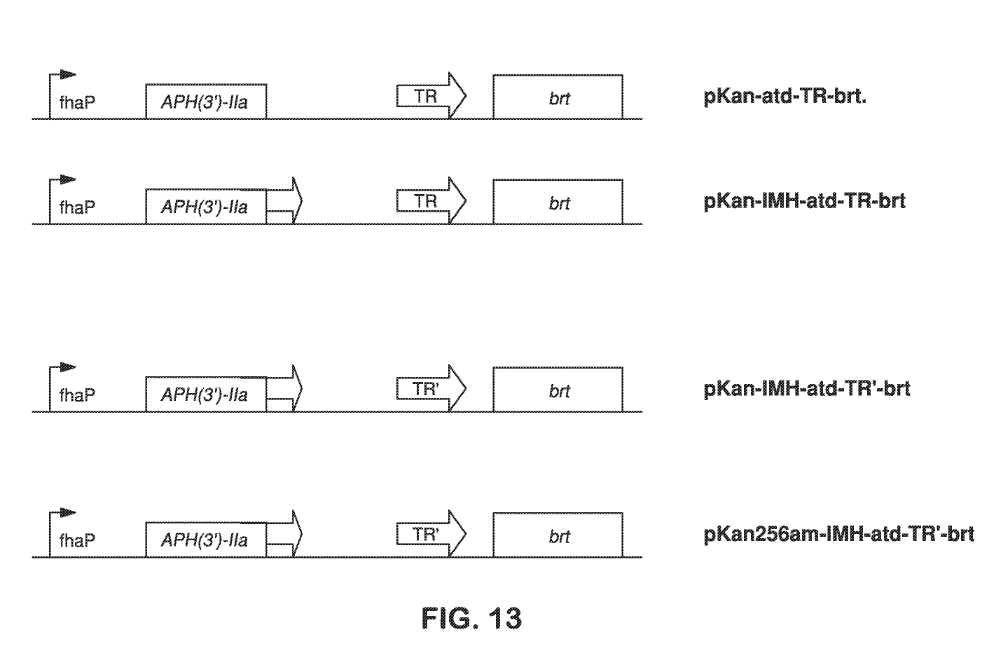

The kanamycin resistance gene was PCR-amplified and digested with restriction enzymes (KpnI and HindIII). The DNA fragment was placed 5′ to the atd-TR-brt region in the plasmid pfhaP-atd-TR-brt. The resulting plasmid is pKan-atd-TR-brt, which carries a deletion of the transcription terminator structure upstream of the atd. (see FIG. 13).

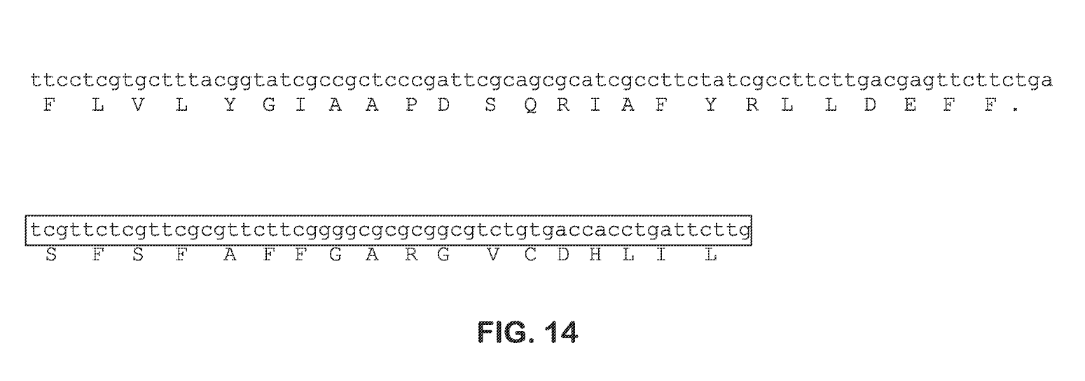

The designed VR region for the kanamycin resistance gene (APH(3′)—IIa includes the last 75 bp in the gene (encoding 25 residues ending with Phe) followed by a stop codon tga and 55 bp from the end of gene mtd. (see FIG. 14). The 55 bp mtd region, shown with a hypothetical encoded peptide sequence, includes 14 bp of the GC rich region (underlined in FIG. 14) followed by the IMH sequence. The mtd region was PCR-amplified with oligos carrying the flanking regions complementary to each side of the insertion position in plasmid pKan-atd-TR-brt at the 5′ end. The PCR product was purified and used as primers for a modified site-directed mutagenesis on plasmid pKan-atd-TR-brt. The resulting plasmid is pKan-IMH-atd-TR-brt (FIG. 13).

The designed TR′ region for kanamycin resistance gene is shown below in alignment with its cognate VR region. A 130 bp region corresponding to the VR is shown with the codon corresponding to Leu243 capitalized. The last 55 bp is the same as the TR region in the BPP-1 DGR region and is capitalized for emphasis.

| | |
|---|---|
| TR′ | aacctcgtgaatTACggtaacgccgctcccgataagcag |
| 243amVR | ttcctcgtgcttTAAggtatcgccgctcccgattcgcag |
| 243resis1VR | tac |
| 243resis2VR | ttc |
| TR′ | cgcatcgccaactatcgccttcttgacaagaacttctga |
| 243amVR | cgcatcgccttctatcgccttcttgacgagttcttctga |
| TR′ | TCGAACTCGAACGCGAACATCGGGGCGCGCGGCGTCTGT |
| 243amVR | TCGTTCTCGTTCGCGTTCTTCGGGGCGCGCGGCGTCTGT |
| TR′ | GCCCATCACCTTCTTG |
| 243amVR | GACCACCTGATTCTTG |

The TR′ region for the kanamycin resistance gene in plasmid pKan-IMH-atd-TR-brt was made by modified site-directed mutagenesis. The final plasmid is pKan-IMH-atd-TR′-brt (FIG. 13) or pKan-TR′. An amber stop codon was introduced into the kanamycin resistance gene at position 243 by site-directed mutagenesis to produce pKan243 am-IMH-atd-TR′-brt (also referred to as pKan243-TR′).

The plasmid was transformed into lysogen 61-11. The lysogen with plasmid pKan-TR′ grew normally in the presence of kanamycin.

Selection of kanamycin resistance with pKan243-TR′ was as follows. A culture of lysogen 61-11 carrying plasmid pKan243-TR′ was grown overnight followed by serial dilution. The dilutions were plated on LB plates with 40 μg/ml kanamycin. The 61-11 hosts harboring kanamycin resistant plasmids that have “repaired” the amber stop codon by adenine-specific mutagenesis, would be expected to form colonies in the presence of kanamycin. Two robust colonies, 243resis1VR and 243resis2VR, from the plate of hosts harboring pKan243-TR′ were isolated, and, their VR regions were amplified and sequenced.

The results are as shown in the box immediately above, where 243resis1VR contained a taa to tac(Tyr) change; tac is the same codon sequence as that in TR′. This indicates that the TR′ sequence was used to substitute for the VR sequence. Stated differently, the change was the result of sequence substitution from the TR′ to the VR.

In 243resis2VR, taa was changed to ttc(Phe), the result of 2 mutations in the same codon. One of the 2 mutagenic events was an A to T change resulting from diversification of the corresponding A in TR′ while the A to C change was a substitution (or homing) from the TR′ template as seen for 243resis1VR. Phe and Tyr have very similar amino acid structures and are both hydrophilic, and the results show that a Tyr or Phe at position 243, which is Leu (also hydrophilic) in the native sequence, was able to restore kanamycin resistance. This suggests that position 243 tolerates a Leu to Tyr or Phe substitution for maintenance or restoration of phosphotransferase function.

As shown by Nurizzo et al. (J. Mol. Biol., 327:491-506, 2003), the C-terminal domain of the kanamycin resistant protein is involved in binding the kanamycin molecule. According to their published crystal structure, the L243 to amber mutation truncates the protein prior to alpha helices 7 and 8. This leads to loss of C-terminal residues 260-264, which form part of the kanamycin-binding pocket. Thus the sequence changes from a stop codon to those in 243resis1VR and 243resis2VR reflect restoration of the kanamycin binding domain of the phosphotransferase.

The above results also indicate that the IMH does not need to be translated for mutagenesis to occur because the IMH follows a tga stop codon in the above kanamycin phosphotransferase constructs. The above described results may also be performed with a trans construct which provides the TR and RT coding sequences under the control of a separate promoter on a second molecule.

### Example 10

### Identification of a DGR from T. denticola

Treponema denticola is a motile, anaerobic spirochete that colonizes the human oral cavity and has been associated with gum disease. There is a 134 base pair identified variable region (VR) located at the 3′ end of open reading frame TDE2269. A corresponding template region (TR) is located 199 base pairs downstream of the VR and 573 base pairs upstream of a reverse transcriptase coding sequence that bears homology (6e-39) to the Bordetella phage reverse transcriptase (brt). The VR and TR differ at 26 positions, with 23 of those differences occurring in the VR at positions that correspond to adenines within the TR. Two of the three positions that do not correspond to adenines may be a part of the IMH signal since they are the most 3′ positions of variability (see below). Also, TDE2269 has a lipoprotein signal sequence (underlined below) indicating that this protein may be exported to the outer membrane. The VR is shown in bolded text below.

TDE2269-329 Amino Acids
MKNTNSKLKTKVLNRAISITALLLAAGVLLTGCPTGQGKSGGGESSEVTP
NTPVDKTYTVGSVEFTMKGIAAVNAQLGHNDYSINQPHTVSLSAYLIGET
EVTQELWQAVMGNNPSHFNGSPAVGETQGKRPVENVNWYQAIAFCNKLSI
KLNLEPCYTVNVGGNPVDFAALSFDQIPDSNNADWDKAELDINKKGFRLP
TEAEWEWAAKGGTDDKWSGTNTEAELKNYAWYGSNSGSKTHEVKKKKPNW
YGLYDIAGNVAEWCWDWRADIHTGDSFPQDYPGPASGSGRVLRGGSWAGS
ADYCAVGERVNISPGVRCSDLGFRLACRP

To confirm variation in the VR corresponding to adenines in the TR, the restriction enzyme HinCII was used in a variability assay to identify a T. denticola VR that differs from the sequenced VR at 25 nucleotide positions. Twenty-one of the 25 differences occur at positions that correspond to adenines within the TR, and one of the remaining four differences appears to be a direct nucleotide transfer (or homing) from the TR as shown below.

The HinCII recognition site is GTYRAC where Y is C or T; and R is A or G. TR stands for Template Region; VR stands for Variable Region; and IV stands for Identified Variant of Variable Region. A portion of presumptive IMH-like and IMH sequences of TR and VR, respectively, are shown in bold type.

TR: CCGCGTCAGGCTCTAACCGTGTTAAACGCGGCGGCAGCTGGAACAA
VR: CCGCGTCAGGCTCTGGCCGTGTTTTACGCGGCGGCAGCTGGGCCGG
IV: ------------------------------------------A-AA
TR: CAACGCGAACAACTGCACTGTAGGCAAACGGAATAACAACAGTCCT
VR: CAGCGCGGACTACTGCGCTGTAGGCGAACGGGTCAACATCAGTCCT
IV: -TA------GGG----A----G---ACC----GT---GG--AC---
TR: GACAACAGGAACAACAATCTTGGCTTCCGCTTGGCTTGTCGGCC
VR: GGCGTCAGGTGCAGCGATCTTGGCTTCCGCCTGGCTTGCCGGCC
IV: ---AA----G---A-CT---------------------------

### Example 11

### Phage Production for DGR Homing Assays

For single-cycle lytic infection, B. bronchiseptica RB50 cells (Liu, M. et al., J. Bacteriol., 186:1503-1517 (2004)) transformed with appropriate donor plasmids were grown overnight at 37° C. in Luria-Bertani (LB) media containing 25 μg/ml of chloramphenicol (Cam), 20 μg/ml streptomycin (Str) and 10 mM nicotinic acid (NA). A 200 μl aliquot of cells was pelleted, rinsed, and resuspended in 1.2 ml Stainer Scholte (SS) medium (Stainer, D. W. and Scholte, M. J., J. Gen. Microbiol., 63:211-220 (1970)) containing 25 μg/ml Cam and 20 μg/ml Str (SS+Cam+Str). Cultures were grown for 3 hours at 37° C. to modulate bacteria to the Bvg+ phase, phages were added at a multiplicity of infection (MOI) of −2.0 and incubated at 37° C. for 1 hour to allow phage absorption. Infected cells were pelleted and resuspended in 1 ml fresh, pre-warmed SS+Cam+Str media and incubated at 37° C. for 3 hours total post phage addition to allow completion of a single cycle of phage development. Progeny phages were harvested through chloroform extraction.

For phage production from lysogens, RB50 derivatives carrying prophage and plasmids of interest were grown and modulated to the Bvg+ phase as in single-cycle lytic infections. Phage production was induced with 2 μg/ml mitomycin for 3 hours at 37° C. Progeny phages were harvested through chloroform extraction.

### PCR-Based DGR Homing Assay

Sequence insertions in TR that are transferred to VR during DGR homing are used as tags for PCR-based detection of homing products. Standard assays were carried out in a volume of 50 μl containing 60 mM Tris-SO₄ (pH 9.1), 18 mM (NH₄)₂SO₄, 2 mM MgSO₄, 200 μM dNTPs, 5% DMSO, 6 ng/μl each of appropriate primers, 0.5 μl Elongase Enzyme Mix (Invitrogen) and ˜2-50×10⁵ copies of phage DNA. PCR reactions were performed as follows: 1× (94° C., 2 minutes); 30-35× (94° C., 30 seconds; 50-55° C., 30 seconds; 72° C., 1 minute); 1× (72° C., 10 minutes); 1× (4° C., hold). 5 to 20 μl samples were analyzed on 2% agarose gels.

### Example 12

### Supplemental Experimental Procedures

Oligonucleotides: List of oligonucleotides used in this study:

| Name | Sequence |
|---|---|
| P1 | 5′CCCTCTAGAGCTCCGGTTGCTTGTGGACG |
| P2 | 5′AGCAAGCTTCCTCGATGGGTTCCAT |
| P3 | 5′ATATCTAGACGTTTTCTTGGGTCTACCGTTTAATGTCG |
| P4 | 5′ATAAAGCTTCGACATTAAACGGTAGACCCAAGAAAA |
| P5 | 5′AAATCTAGATCTGTCTGCGTTTGTGTT |
| P6 | 5′AGCAAGCTTAGCACAGGAACACAAACG |
| P7 | 5′CCCTCTAGAATTCCAGGCGCTGGCTTTC |
| P8 | 5′AGCGGATCCGAAGCAGGACAGAACCG |
| P9 | 5′AGCGGATCCACCTATTGAGGAAAGGC |
| P10 | 5′AAATCTAGACGCTGCTGCGCTATTCGGCGGC |
| E1s | 5′ATATCTAGACGGGCCGTCGAAGTCGACGTTTTCTTG |
| E2a | 5′ATAAAGCTTCGCGTTCGAGGTCGACATTAAACGG |
| tdE2 | 5′AGGTCGACATTAAACGGT |

### Bacterial Strains and Phages

B. bronchiseptica RB50, RB53 Cm, and RB54 have been previously described (Liu, 5M. et al., J. Bacteriol., 186:1503-1517 (2004)). Strain RB50ΔrecA was constructed by deleting the entire recA ORF via allelic exchange. The BPP-1d lysogen was constructed from an RB50 BPP-1 lysogen (ML6401) (Liu, M. et al., J. Bacteriol., 186:1503-1517 (2004)) by deleting sequences from the 5′ end of TR to position 882 of brt. BPP-1dΔVR1-99 and BPP-1dΔVR1-991MH* lysogens were constructed from BPP-1d, and both have a deletion of VR from position 1 to 99. In the latter construct, the IMH in VR was replaced by IMH* from TR. Phage BPP-1 has been described previously (Liu, M. et al., J. Bacteriol., 186:1503-1517 (2004)) and derivative phages are produced from the above lysogens.

### Plasmid Constructs

Plasmid donors used in DGR homing assays were derived from pMX1, which expresses the BPP-1 atd, TR, and brt loci under control of an fhaB promoter (Xu et al., in preparation). pMX1 includes a pBBR1 replication origin and confers chloramphenicol resistance (Antoine, R. and Locht, C., Mot Microbiol, 6:1785-1799 (1992)). pMX1/SMAA is an RT-deficient mutant of pMX1 with the YMDD box in the brt ORF replaced with amino acid residues SMAA (Liu, M. et al., Science, 295:2091-2094 (2002)).

pMX1b is a derivative of pMX1 with the Sall restriction site in the polylinker between the jhaB promoter and atd eliminated via Sall restriction, filling-in by T4 DNA polymerase and religation. Plasmids pMX-TG1a, pMX-TG1b and pMX-TG1c are derivatives of pMX1b with a 36 bp sequence (5′-GTCGACGTTTTCTTGGGTCTACCGTTTAATGTCGAC) inserted at TR positions 19, 47 and 84, respectively. The 36 bp sequence includes 24 bp ligated exons of the phage T4 td group I intron flanked by Sall sites.

Plasmid pMX-td is derived from pMX-TG1c with the tdA 1-3 intron (a splicing-competent derivative of phage T4 td group I intron lacking most of the intron ORF) inserted between the exons (Cousineau, B. et al., Cell, 94:451-462 (1998)). The intron was inserted in the same orientation as that of TR. Plasmids pMX-td/P6M3 and pMX-td/ΔP7.1-2a are derivatives of pMX-td with splicing-defective td introns. pMX-td/P6M3 has G78C and C79G substitutions, while pMX-td/ΔP7.1-2a carries a A877-908 deletion (Mohr, G. et al., Cell, 69:483-494 (1992)). Plasmid pMX-td− has the td intron and its flanking exons inserted in the reverse orientation.

pMX-AvAp was constructed to facilitate introduction of mutations into TR and its flanking regions and was derived from pMX1b through site-directed mutagenesis. Sequences from atd position 336 to brt position 186 were replaced with the sequence 5′-CCTAGGCCGCGGGCCC to introduce AvrII and Apal sites. To eliminate the AvrII and Apal sites in the polylinker in front of the atd gene, sequence 5′-CCTAGGTACCGGGCCC was replaced with 5′-CCTAGATATCGGTCTC. Plasmid pMX-TG1c/AA was derived from pMX-AvAp and is essentially the same as pMX-TG1c, with the exception of Avrll and Apal sites in atd and brt which were introduced by silent mutations. Silent mutations were verified not to affect DGR homing (pMX-TG1c/AA in FIG. 28).

Plasmids used for TR internal deletion analysis in FIG. 2 were constructed from the pMX-AvAp vector: pMX-ΔTR1-84, pMX-ΔTR11-84, pMX-ΔTR23-84 and pMX-ΔTR33-84 include 50 bp of the 3′-end TR (3′ boundary at TR position 85), while having 0, 10, 22 and 32 by of the 5′-end TR, respectively; pMX-ΔTR33-97 and pMX-ΔTR33-113 have 32 by of the 5′-end TR, while having 38 and 21 bp of the 3′-end TR, respectively. Between the deletion junctions, all the constructs have a 32 bp sequence (5′-AGATCTGTCTGCGTTTGTGTTCCTGTGCTAGC) inserted as a tag (TG2) to facilitate DGR homing analysis. pMX-FL was constructed as a positive control, with TG2 inserted between positions 84 and 85 of the full-length TR.

Additional TR internal deletion constructs pMX-ΔTR20-47, pMX-ΔTR20-84 and pMX-ΔTR48-84 were derived from pMX-TG1a, pMX-TG1b and pMX-TG1c, and contain deletions between the insertions of TG1a and TG1b, TG1a and TG1c, and TG1b and TG1c, respectively. At the deletion junctions, the 36 bp TG1 tag was inserted to facilitate DGR homing assays.

Plasmids for silent mutation scanning to determine regions of the RNA transcript important for DGR homing were constructed from pMX-AvAp and are identical to pMX-TG1c/AA except for the silent mutations. Plasmids pMX-A1, pMX-A2 and pMX-A3 contain silent mutations in the atd ORF and have the 3′-end atd sequences 5′-ATTGCCCGC, 5′-GTGAATCGC and 5′-GCTGGGAAA replaced by 5′-ATTGCGAGG, 5′-GTGAACAGG and 5′-GCAGGCAAG, respectively. The brt ORF can potentially be extended at the 5′ end to sequences upstream of atd. Plasmids pMX-T1, pMX-T2 and pMX-T3 contain silent mutations upstream of the brt ORF, and have the TR sequences 5′-CTGCTGCGC, 5′-TCGGGGCGC and 5′-CCCATCACC replaced by 5′-CTGCTCAGG, 5′-TCGGGAAGG and 5′-CCAATAACA, respectively. Plasmids pMX-T4, pMX-T5 and pMX-T6 contain silent mutations in sequences upstream of the brt ORF, and have the sequences 5′-CTTTCCTCA, 5′-ACGTCGATT and 5′-ACTTCTTCA in the spacer between TR and brt replaced by 5′-CTTAGTAGC, 5′-ACCAGCATA and 5′-ACTAGCAGC. Plasmids pMX-T7, pMX-T8 and pMX-T9 contain silent mutations in the brt ORF, and have the brt sequences 5′-AATCTGCTC, 5′-AAGCGCCGG and 5′-CTGCTGGCC replaced by 5′-AACCTCCTG, 5′-AAGAGAAGA and 5′-CTCCTCGCG.

Plasmids for marker transfer/coconversion studies were also constructed from pMX-AvAp and include pMX-TRC1T, pMX-TRC6T, pMX-TRC11T; pMX-TRC16T, pMX-TRC22T, pMX-TRC43T, pMX-TRC81T, pMX-TRC85T, pMX-TRC91T, pMX-TRC97T, pMX-TRC100T, pMX-TRC105T, pMX-TRC 107T, pMX-TRC109T, pMX-TRC112T, pMX-TRC115T, pMX-TRC120T and pMX-TRC125T. They are identical to pMX-TG1c/AA except for the C to T substitutions at the indicated TR positions.

Plasmid pMX-M50 was derived from pMX-ΔTR23-84 and contains a 50 bp mtd sequence upstream of VR (mtd positions 952 to 1001) inserted at the BgIII site downstream of TR position 22. Plasmid pMX-M50/SMAA is a Brt-deficient derivative of pMX-M50, with the essential YMDD box in the brt ORF replaced by SMAA.

### Phage DNA Purification

To remove bacterial chromosomal and plasmid DNAs, phage lysates were treated with 50 μg/ml DNase I (Sigma) and 2.5 ng/ml micrococcal nuclease (Roche) in 10 mM Tris-HCl (pH 7.5), 1.0 mM CaCl₂, and 10 mM MgCl₂ at 37° C. overnight. Aliquots were titrated to determine phage concentrations. Reactions were terminated by the addition of 5.0 mM EGTA and 5 ng/ml protease K followed by incubation at 37° C. for >15 minutes. Protease K was heat-inactivated at 70° C. for >15 minutes. Samples were extracted with phenol-chloroform-isoamyl alcohol (Φ-CIA, 25:24:1) followed by chloroform extraction. For Mtd-defective phages, phage DNA concentrations were determined through quantitative PCR assays.

### RNA Isolation for td Intron Splicing Assays

Total RNAs were isolated from RB50 cells transformed with appropriate plasmids following induction in Stainer Scholte (SS) media containing 25 μg/ml of chloramphenicol (Cam), 20 μg/ml streptomycin (Str) (SS+Cam+Str) for 6 hours to express donor constructs. RNAs were isolated with trizol/CHCl₃ extraction and Φ-CIA (phenolchloroform-isoamyl alcohol; 25:24:1) extraction, followed by ethanol precipitation. Subsequently, RNA samples were treated with Turbo DNase I (0.033 u/μl; Ambion) in 1× Turbo DNase I buffer at 37° C. for 40 minutes to eliminate DNA contamination, Samples were then treated with protease K (17 μg/ml final concentration) at 37° C. for 5 minutes to eliminate Turbo DNase I. RNAs were prepared by Φ-CIA extraction and ethanol precipitation.

Analysis of td Intron RNA Splicing in B. bronchiseptica by RT-PCR and Primer-Extension-Termination Assays

To analyze td intron RNA splicing by RT-PCR, cDNA products were first generated with primer P8 in a 20 μl reaction containing 50 mM Tris-HCl (pH 8.3), 75 mM KCl, 3 mM MgCl2, 5 mM DTT, 5% DMSO, 5 μg total RNAs, 10 u/μl Superscript III RT (Invitrogen) and 1.85 u/μl RNase Inhibitor (Amersham) at 50° C. for 1 hour. Superscript III RT was then heat-inactivated at 70° C. for 15 minutes. Subsequently, 2 μl cDNA products were amplified by PCR in a 50 μl reaction containing 60 mM Tris-SO₄ (pH 9.1), 18 mM (NH₄)₂SO₄ and 2 mM MgSO₄, 200 μM dNTPs, 5% DMSO, 6 ng/μl primers P9 and P10 each, and 0.5 μl Elongase Enzyme Mix (Invitrogen). PCR reactions were performed under the following condition: 1× (94° C., 2 minutes); 15× (94° C., 30 seconds; 50° C., 30 seconds; 72° C., 1 minute); 1× (72° C., 10 minutes); 1× (4° C., hold).

Relative amounts of precursor and spliced RNAs were measured through primer-extension-termination assay (Zhang, A. et al., RNA, 1:783-793 (1995)) using Superscript III RT (invitrogen) and a td exon 2 primer (tdE2).

### Phage Tropism Switching Assay

Progeny phages for tropism switching assays were generated from phage BPP-1d through single-cycle lytic infection of B. bronchiseptica RB50 or RB50ΔrecA cells transformed with appropriate donor plasmids. To determine phage tropism switching frequencies, progeny phages were serially diluted and plaque-forming units on RB54 (Bvg⁻) and RB53 Cm (Bvg⁺) cells were determined. Phage tropism switching frequencies were defined as the ratio of plaque-forming units on RB54 cells (Bvg⁻ tropism) vs. those on RB53 Cm cells (Bvg⁺ tropism).

### Total Nucleic Acid Purification

BPP-1dΔVR1-99 and BPP-1dΔVR1-99IMH* lysogens transformed with donor plasmids were grown overnight, modulated to the Bvg+ phase, and induced with 2 μg/ml mitomycin for 2 hours. Cells were precipitated, resuspended in 10 mM Tris-HCl (pH 8.0), 100 mM NaCl, 1 mM EDTA and 0.1% SIDS. Total nucleic acids were purified by (D-CIA extraction followed by chloroform extraction and ethanol precipitation. Pellets were resuspended in 10 mM Tris-HCl (pH 7.5), 1 mM EDTA and 10 ng/μl protease K. Following incubation at 37° C. for 30 minutes, samples were extracted with (Φ-CIA and precipitated with ethanol.

All references cited herein are hereby incorporated by reference in their entireties, whether previously specifically incorporated or not. As used herein, the terms “a”, “an”, and “any” are each intended to include both the singular and plural forms.

Having now fully described this invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A single recombinant nucleic acid molecule or pair of nucleic acid molecules comprising
a variable region (VR) operably linked to a donor template region (TR)
wherein said TR is operably linked to a reverse transcriptase (RT) coding sequence and is a template sequence that directs site-specific mutagenesis of said VR, and
wherein the TR and RT coding sequence are heterologous to each other.

2. The molecule of claim 1, wherein the sequence of said TR is an imperfect direct repeat of the sequence in said VR due to the substitution of one or more adenine nucleotides in said TR, or substitution of one or more non-adenine nucleotides in VR by adenines in TR, or substitution of VR adenine nucleotides by non-adenine nucleotides in TR.

3. The molecule of claim 1, wherein said VR is all or part of a sequence encoding a binding partner of a target molecule.

4. The molecule of claim 3, further comprising all of the sequence encoding said binding partner, wherein said VR is optionally the 3′ portion of said sequence encoding said binding partner.

5. The molecule of claim 3, wherein said binding partner binds a cell surface molecule, a hormone, a growth or differentiation factor, a receptor, a ligand of a receptor, a bacterial cell wall molecule, a viral particle, an immunity or immune tolerance factor, or an MHC molecule.

6. The molecule of claim 3, wherein said binding partner is a bacteriocin.

7. The molecule or pair of molecules of claim 1, wherein said TR and RT coding sequence are transcribed under the control of a heterologous promoter.

8. A cell containing the molecule or pair of molecules of claim 1.

9. A method of preparing the single molecule of claim 1, said method comprising
operably linking a first nucleic acid molecule comprising said VR to a second nucleic acid molecule comprising said TR such that said TR is a template sequence that directs site specific mutagenesis of said VR.

10. A method of preparing one of the molecule or pair of molecules of claim 7, said method comprising
operably linking a heterologous promoter sequence to a nucleic acid molecule comprising said TR and RT coding sequence.

11. A method of site-specific mutagenesis of a nucleic acid sequence of interest, said method comprising
obtaining a nucleic acid molecule or pair of molecules of claim 1 wherein said VR comprises said nucleic acid sequence of interest and said TR is an imperfect or perfect repeat of said sequence of interest,
wherein said TR is a template sequence operably linked to said sequence of interest to direct site-specific mutagenesis of the sequence, and wherein said TR is an imperfect repeat due to the substitution of one or more adenine nucleotide for a non-adenine nucleotide in said sequence of interest or visa versa; and
allowing said nucleic acid molecule to be expressed in a cell such that one or more nucleotide positions of said sequence of interest is substituted by a different nucleotide.

12. The method of claim 11, wherein more than one nucleotide position of said sequence of interest is substituted.

13. The method of claim 11, wherein said sequence of interest encodes all or part of a binding partner of a target molecule.

14. The method of claim 13, wherein the binding properties of said binding partner are altered.

15. An isolated nucleic acid molecule comprising
a donor template region (TR) and an operably linked reverse transcriptase (RT) coding sequence,
wherein the TR and RT coding sequence are heterologous to each other.

16. The molecule of claim 15, wherein the molecule is isolated from a bacteriophage, a prophage of a bacterium, a bacterium, or a spirochete.

17. A plurality or library of nucleic acid molecules according to claim 1.

18. The plurality or library of claim 17, wherein the VR has undergone diversification directed by the TR.

19. A method of identifying initiation of mutagenic homing (IMH) sequences, said method comprising
identifying an RT coding sequence in a genome of an organism;
searching the coding strand within about 5 kb of the RT ORF and identify an IMH-like sequence containing an 18-48 nucleotide stretch of adenine-depleted DNA; and
a) using the putative IMH-like sequence to search genome-wide for a closely-related putative IMH and compare the DNA sequences located 5′ to the IMH-like and putative IMH sequences to find TR and VR regions, respectively; or
b) using the sequence of the DNA located 100-350 base-pairs long 5′ to the IMH-like sequence to identify a putative TR, and use all or parts of this TR and IMH-like sequence to search genome-wide for a matching putative VR and IMH sequence.

20. The method of claim 19 wherein said RT coding sequence is identified by searching for one or both amino acid sequences IGXXXSQ (SEQ ID NO:33) or LGXXXSQ (SEQ ID NO:34); or
wherein the IMH-like, or IMH, sequence contain a conserved sequence selected from TCGG, TTTTCG, or TTGT; or
wherein the identified TR and VR sequences can be between about 100-350 base-pairs long and should be more than about 80% homologous, with the majority of differences being at the locations of the adenines bases in the TR.

21. A method of site-specific mutagenesis of a nucleic acid sequence of interest, said method comprising:
obtaining a nucleic acid molecule comprising a donor template region (TR) and a variable region (VR), wherein said TR or VR or operably linked reverse transcriptase (RT) coding region is isolated from Vibrio harveyi ML phage, Bifidobacterium longum, Bacteroides thetaiotaonicron, Treponema denticola, or a cyanobacterial diversity generating retroelements (DGRs), and
allowing said nucleic acid molecule to be expressed in a cell such that one or more nucleotide positions of said VR is substituted by a different nucleotide.

22. The method of claim 21, wherein said DGR is isolated from Trichodesmium erythraeum #1, Trichodesmium erythraeum #2, Nostoc PPC ssp. 7120 #1, Nostoc PPC ssp. 7120 #2, or Nostoc punctiforme.

23. The molecule of claim 1, wherein the 3′ end of the VR comprises about a 14 base pair element consisting of G and C residues, not A residues.

24. The molecule of claim 23, wherein about 4 to about 12 base pairs of the VR 5 upstream, of and within about 350 base pairs of the base pair element have sequence homology with about 4 to about 12 base pairs of the TR.

25. The molecule of claim 1, wherein the VR comprises an initiation of mutagenic homing (IMH) sequence at its 3′ end.

26. The molecule of claim 1, wherein the TR consists essentially of about 10 to about 19 base pairs at its 5′ end and about 38 base pairs at its 3′ end.

27. The molecule of claim 1, wherein the TR is further extended upstream of the TR comprising the 3′ end of an atd region and the nucleotides between the TR and atd region; and the TR is further extended downstream of the TR comprising the 5′ end of a brt region and the nucleotides between the TR and brt region.

28. The molecule of claim 1, wherein the TR comprises an initiation of mutagenesis homing-like (IMH*) sequence at its 3′ end.

29. The method of claim 11, wherein the function of the TR comprises an RNA intermediate.

30. The method of claim 11, which is RecA-independent.
